(19) United States
(12) Patent Application Publication
(10) Pub. No.: US 2014/0178305 A1
(43) Pub. Date: Jun. 26, 2014

(54) IDENTIFICATION AND USE OF ANXIOLYTIC COMPOUNDS

(71) Applicant: CALIFORNIA INSTITUTE OF TECHNOLOGY, PASADENA, CA (US)

(72) Inventors: David J. Anderson, Altadena, CA (US); Sophia Vrontou, Pasadena, CA (US)

(21) Appl. No.: 14/135,102

(22) Filed: Dec. 19, 2013

Related U.S. Application Data
(60) Provisional application No. 61/740,285, filed on Dec. 20, 2012.

(51) Int. Cl. A61K 49/00; G01N 33/50

(52) U.S. Cl. CPC A61K 49/0004; G01N 33/5058
USPC 424/9.2; 435/29; 514/17.7; 514/44 R

(57) ABSTRACT

The present disclosure provides methods and compositions that can be used to identify anxiolytic compounds, and to treat anxiety in a subject. Methods and compositions for identifying activating stimuli for sensory neurons, for example MrgprB4⁺ neurons, are also disclosed herein.

### RELATED APPLICATIONS

The present application claims priority under 35 U.S.C. §119(e) to U.S. Provisional Application No. 61/740,285, filed on Dec. 20, 2012, which is herein expressly incorporated by reference in its entirety.

### STATEMENT REGARDING FEDERALLY SPONSORED R&D

This invention was made with government support under Grant 5PO1NS-48499 and 5R01 NS023476 awarded by the National Institutes of Health. The government has certain rights in the invention.

### REFERENCE TO SEQUENCE LISTING

The present application is being filed along with a Sequence Listing in electronic format. The Sequence Listing is provided as a file entitled CALTE_{—}095_SEQLIST.TXT, created Dec. 19, 2013, which is 4 kb in size. The information in the electronic format of the Sequence Listing is incorporated herein by reference in its entirety.

### BACKGROUND

1. Field of the Application

The present application relates generally to methods of determining activating stimuli for sensory neurons, and methods and compositions for the identification and use of compounds with anxiolytic activities.

2. Description of the Related Art

Stroking of the skin produces pleasant sensations that can occur during social interactions with conspecifics, such as grooming. Despite numerous physiological studies, molecularly defined sensory neurons that detect pleasant stroking of hairy skin in vivo have not been reported. Previously, a rare population of unmyelinated sensory neurons was identified in mice that express the G-protein-coupled receptor MrgprB4. Dong et al., Cell 106:619-632 (2001); Zylka et al. Proc. Natl. Acad. Sci. USA 100:10043-10048 (2003). These neurons exclusively innervate hairy skin with large terminal arborizations that resemble the receptive fields of C-tactile (CT) afferents in humans. Electrophysiological analysis of MrgprB4⁺ neurons in culture has failed to reveal responses to any thermal or mechanical stimuli tested. Liu et al., Nature Neuroscience, 10(8):946-948.

### SUMMARY

Some embodiments disclosed herein provide a method of identifying compounds having anxiolytic activities. In some embodiments, the method includes: (a) providing a candidate compound; (b) testing the candidate compound for its ability to activate MrgprB4⁺ neurons; and (c) testing the candidate compound for its activity to stimulate positive valence behavior in a subject if the candidate compound activates MrgprB4⁺ neurons in step (b).

In some embodiments, step (b) is carried out in vitro. In some embodiments, step (b) is carried out in a skin-nerve culture. In some embodiments, the candidate compound is an MrgprB4 agonist. In some embodiments, step (b) is carried out in vivo. In some embodiments, the candidate compound is administered to the subject via injection. In some embodiments, the candidate compound is injected into spinal cord of the animal or via peripheral injection into the skin of the subject. In some embodiments, step (b) comprises performing calcium imaging in MrgprB4⁺ neurons.

In some embodiments, step (c) comprises testing the candidate compound using a conditioned place preference assay. In some embodiments, step (c) comprises determining conditioned place aversion. In some embodiments, step (c) comprises applying the candidate compound peripherally on the subject.

In some embodiments, the candidate compound is applied topically on the subject. In some embodiments, the candidate compound is in a topical composition selected from the group consisting of lotion, cream, foam, ointment, gel, transdermal patch, powder, and spray. In some embodiments, the candidate compound is a small molecule, peptide, or nucleic acid.

Some embodiments provide a method of treating anxiety in a subject. In some embodiments, the method includes identifying a subject suffering from anxiety; and administering to the subject an effective amount of an activator for MrgprB4⁺ neurons.

In some embodiments, the method additionally includes the step of identifying an activator for MrgprB4⁺ neurons. In some embodiments, the activator for MrgprB4⁺ neurons is an agonist for MrgprB4⁺ receptor. In some embodiments, the activator for MrgprB4⁺ neurons is topically administered to the subject. In some embodiments, the activator for MrgprB4⁺ neurons is a small molecule, a peptide, or a nucleic acid. In some embodiments, the anxiety is caused by itching or pain.

Also disclosed herein are methods for identifying activating stimuli for sensory neurons. In some embodiments, the method includes applying a stimulus to a subject, wherein the subject has a population of a subset of sensory neurons; and performing two-proton calcium imaging to determine activation of the subset of sensory neurons. In some embodiments, the sensory neurons are MrgprB4⁺ neurons. In some embodiments, the population of a subset of sensory neurons is genetically modified.

In some embodiments, the genetic modification is carried out by intra-peritoneally injecting a viral vector to neonatal pups of the subject. In some embodiments, the viral vector is derived from adeno-associated virus of serotype 8 (AAV8). In some embodiments, the viral vector comprises two portions of MrgprB4 open reading frame, wherein the two portions of MrgprB4 open reading frame are separated by a nucleic acid sequence encoding one or more marker genes.

In some embodiments, the neonatal pups of the subject inducibly express Cre recombinase in ganglia. In some embodiments, the neonatal pups of the subject inducibly express Cre recombinase in MrgprB4⁺ neurons.

In some embodiments, the stimulus is a mechanical stimulus, a thermal stimulus, a chemical stimulus, or a combination thereof. In some embodiments, the stimulus is applied centrally or peripherally to the subject. In some embodiments, the stimulus is pinching, massaging, grooming, stroking, or brushing.

In some embodiments, two-proton calcium imaging is carried out on the skin of the subject.

### BRIEF DESCRIPTION OF THE DRAWINGS

FIGS. 1A-1N relate to in vivo calcium imaging in genetically defined subsets of primary sensory neurons. FIG. 1A is a schematic illustration of AAV infection. LSL=loxP-STOP-loxP cassette. FIGS. 1B-1E depict mGCaMP3.0 expression in somata (FIG. 1B, FIG. 1C) and central afferent fibres (FIG. 1D, FIG. 1E) of MRGPRD⁺ (FIG. 1B, FIG. 1D) or MRGPRB4⁺ (FIG. 1C, FIG. 1E) neurons in adult mice. The dashed lines indicate lateral margin of spinal cord. Scale bar: 50 μm (FIG. 1B) and 45 μm (FIG. 1D). FIG. 1F is a schematic illustrating imaging preparation. The components are not to scale. FIG. 1G-1N depict calcium transients in the central projections of MrgprD⁺ (FIGS. 1G, 1I, 1K, 1M) or MrgprB4⁺ (FIGS. 1H, 1J, 1L, 1N) neurons, evoked by direct application of KCl to the spinal cord (FIGS. 1I, 1J) or (in a different animal) peripheral injection of α,β-methylene ATP (FIGS. 1K, 1L). The rectangles in FIG. 1G and FIG. 1H indicate Regions of Interest (ROIs) used in FIGS. 1I and 1J, respectively; and the boxes on the upper right corner of Figures G and H are regions for background subtraction. Scale bar: 40 μm (FIG. 1G) and 20 μm (FIG. 1H). The arrow (in FIGS. 1I-1L) indicates time of stimulus delivery. FIGS. 1M and 1N depict the quantification of peak ΔF/F values before (open bars) versus after (filled bars) stimulation. **P<0.01; ***P<0.001. All data are mean±s.e.m. and “L” is lateral, “M” is medial, and “R” is rostral.

FIGS. 2A-2J relate to activation of MrgprD fibres by pinching. FIGS. 2A and 2B are schematics illustrating pinching (2A) and stroking (2B) stimuli. FIG. 2C depicts GCaMP3.0 fluorescence in one imaging frame during stimulation and ROIs used for imaging in FIGS. 2D-21. In FIG. 2C, the rectangle in the upper right shows the region for background subtraction, scale bar=9 μm. The four boxes in the center of FIG. 2C represent four Regions of Interest (ROIs). FIG. 2D depicts superimposed traces from the four boxes shown in FIG. 2C in a single trial consisting of four pinch stimuli corresponding to response curves in FIGS. 2D-2G. FIG. 2E depicts the average response to pinching in a single mouse (n=4 trials, 7 stimuli total), further discussed herein with respect to FIGS. 9A-9E. FIG. 2F depicts a response to four brushing stimuli (vertical bars) delivered to pinch-sensitive digit (FIG. 2D), in corresponding ROIs from FIG. 2C. This is also shown and discussed further in FIG. 13G-L and the associated text. FIG. 2G depicts an average response to brushing (n=2 trials, 7 stimuli total). FIGS. 2H and 2I depict MPI ΔF/Fₚₑₐₖ (upper portions of respective figures) or integrated area (lower portions) from curves in FIGS. 2E and 2G, respectively. Open and filled bars are 5 frames before and 40 frames after stimulus delivery, respectively. In FIG. 2, for calculating ΔF/F [(Fav−F₀)/F₀], F₀ is the average of the first 10 frames of the recording period. Since the baseline gradually declines during a trial (FIGS. 2D and 2F), some ΔF/F values are <0 in unresponsive ROIs or in the immediate pre-stimulus period (left of dashed lines in FIGS. 2E and 2G). FIG. 2J shows MPI ΔF/Fₚₑₐₖ in the third ROI from the left on FIG. 2C (top portion of FIG. 2J) and MPI ΔF/Fₚₑₐₖ in the second ROI from the left on FIG. 2C (bottom portion of FIG. 2J), respectively, from each of three mice. The open and filled bars are 5 frames before and 40 frames after stimulus delivery, respectively. Further detail is shown in FIG. 20 and discussed in the associate text. For FIGS. 2A-2J, *P<0.05, **P<0.01, and ***P<0.001. All data are mean±s.e.m.

FIGS. 3A-3J relate to activation of MrgprB4⁺ fibres by stroking. FIGS. 3A and 3B are schematics illustrating brushing (FIG. 3A) and pinching (FIG. 3B) stimuli. FIG. 3C depicts GCaMP3.0 fluorescence in one imaging frame during stimulation and ROIs used for imaging in FIGS. 3D-3I. The rectangle on the lower right of FIG. 3C is the region used for background subtraction. Scale bar=8.5 μm. The four boxes in the center of FIG. 3C represent four Regions of Interest (ROIs) which correspond to curves depicted in FIGS. 3D-3G, and to bar groups 324, 326, 328 and 329, respectively, in FIGS. 3H and 3I. FIG. 3D depicts superimposed traces from the ROIs 304, 306, 308 and 309 in FIG. 3C in a single trial of three brush stimuli (vertical bars). FIG. 3E depicts an average response to brushing from a single mouse (n=5 trials, ˜3-6 stimuli per trial). See also FIGS. 9G-9J. FIG. 3F depicts a response to five pinching stimuli (vertical bars) in brush-sensitive region (FIG. 3D), in the corresponding ROI (FIG. 3C). This is further discussed herein with respect to FIGS. 13A-13F. FIG. 3G depicts an average response to pinching from the same animal (n=2 trials, 10 stimuli total). FIGS. 3H and 3I depict MPI ΔF/Fₚₑₐₖ (upper portions of the respective figures) or integrated area (lower portions) calculated from the curves in FIGS. 3E and 3G, respectively. Open and filled bars are 5 frames before and 20 frames after stimulus delivery, respectively, where “NS” indicates not significant. FIG. 3J depicts MPI ΔF/Fₚₑₐₖ in the two ROIs, from each of three independent mice. Open and filled bars are as in the panels shown in FIGS. 3H and 3I. Further detail is shown in FIG. 21 and discussed in the associated text. In FIGS. 3A-3J, **P<0.01 and ***P<0.001. All data are mean±s.e.m.

FIGS. 4A-4J show that activation of MrgprB4 neurons promotes conditioned place preference. FIGS. 4A and 4B are schematics of experiment (FIG. 4A) and CPP apparatus (FIG. 4B). I.N.P. and I.P. indicate initially non-preferred and preferred chambers, respectively (In the schematic of FIG. 4C, indicated by “pre-test”). FIG. 4C: the top of FIG. 4C depicts absolute time (s) in each chamber before (open bars; ‘pre’) versus after (filled bars; ‘post’) conditioning for the experimental group. “Train.drug” in FIG. 4C indicates CNO or saline paired with the indicated chamber. The bottom of FIG. 4C is a schematic of experimental design, where “cham.” indicates chamber. FIG. 4D shows the time in I.N.P. chamber for experimental (replotted from FIG. 4C for direct comparison) and control groups. In FIGS. 4C and 4D, **P<0.01, ***P<0.001, and “NS” is not significant. In FIGS. 4C and 4D, detection of a significant interaction (in FIG. 4C, F(2,42)=22.29, p<0.0001; in FIG. 4D, no significant interaction); and/or main effect (in FIG. 4C, F(2,42)=45.05, p<0.0001; in FIG. 4D, F(1,43)=6.355, p=0.01) by ANOVA was followed by a Bonferroni post-hoc test. Further detail is given in FIGS. 17A-17F and the associated text. FIGS. 4E-4I depict the difference scores ((time in indicated chamber after training)−(time in chamber before training)) for experimental (in FIG. 4E, n=15) and control (For FIGS. 4F-I, n=9, 6, 8, and 10, respectively) groups. FIG. 4J shows a comparison of mean difference scores for the I.N.P. chamber for the experimental (FIG. 4E) and the pooled control (FIGS. 4F-4I) groups. There was no significant difference between control groups. In FIGS. 4E-4J, *P<0.05, ** P<0.01 and *** P<0.001. All data are mean±s.e.m.

FIGS. 5A-5D depict generation of MrgprB4 knockout mice. FIG. 5A depicts a targeting construct containing the m-tdTomato-2A-NLSCre-frt-neo-frt cassette, illustrated in the upper portion of FIG. 5A, was designed to replace the entire open reading frame (ORF) of MrgprB4 (the large arrow represents the MrgprB4 locus) following homologous recombination. FIGS. 5B-5D shows southern blot results, confirming occurrence of the homologous recombination event. Genomic DNA from wild type (wt) and MrgprB4^{tdTomato-2A-Cre/+} heterozygous mice was digested, Southern blotted and hybridized with probes shown in FIG. 5A. The sizes are in kb, and the bands shown on FIGS. 5B-5D were predicted by correct homologous recombination event.

FIGS. 6A-6H depict the specificity and efficiency of the neonatal virus injections. FIGS. 6A-6C depict visualization of EGFP transgene expression (anti-EGFP) and viral hrGFP expression in the DRG of adult MrgprD-EGFPCre mice injected neonatally with a Cre-dependent AAV8 virus expressing hrGFP. The signal outside of the DRG in FIG. 6A (the dashed outline) is autofluorescence, pseudocolored light speckles 602. FIGS. 6D-6F depict similarly prepared mice showing expression of viral hrGFP expression in ganglia across the rostro-caudal axis. Scale bars: 55 μm (FIGS. 6A-6C) and 35 μm (FIGS. 6D-6F). No expression of hrGFP was detected in wild-type mice injected with the Cre-dependent AAV8:hrGFP (not shown). FIGS. 6G-6H are histograms showing the specificity and efficiency of Cre-dependent virus expression in adult MrgprD-EGFPCre (FIG. 6G) and MrgprB4-tdTomato-2A-Cre (FIG. 6H) mice, following neonatal i.p. injections with Cre-dependent hrGFP and Cre-dependent GCaMP3 virus respectively. LSL denotes loxP-STOP-loxP cassette. In the case of MrgprD-EGFPCre mice, Cre-dependent AAV8:hrGFP was used rather than GCaMP3.0 to enable independent antibody staining of the transgene (EGFP) and viral reporter (hrGFP).

FIGS. 7A-7F depict activation of MrgprD⁺ fibers by α,β-methyl ATP application to the spinal cord. FIG. 7A is a schematic illustrating application of chemical solutions to the spinal cord (not to scale). FIGS. 7B and 7C show an application of imaging solution (arrow in FIG. 7C) did not evoke calcium transient in the same ROI as used for imaging of KCl responses (FIG. 7Bb; cf. FIGS. 1I and 1J). FIGS. 7D and 7E show that an application of α,β-methyl ATP to the spinal cord of MrgD mice induced a strong calcium response (FIG. 7E). The ROI used for imaging is the rectangle in the center of FIG. 7B, and the rectangle on the upper left of FIG. 7B is the region used for background subtraction (FIGS. 7B and 7D). FIG. 7F is a bar graph of peak ΔF/F values before (“pre”) vs. after (“post”) stimulation, where n=2, mean±range. The scale bars in FIGS. 7B and 7D are 40 and 5.5 μm, respectively.

FIGS. 8A-8F depict imaging activity after peripheral injection of capsaicin in MrgprB4-Cre×Rosa-loxPSTOPloxP-TRPV1 mice. FIG. 8A is a schematic illustrating peripheral injection of capsaicin or α,β-methylene ATP into hairy skin of hindlimb. FIG. 8B shows calcium transients in the central afferent fibers of mice expressing GCaMP3.0 and TRPV1 receptor in MrgprB4⁺ neurons, evoked by peripheral injection of capsaicin. FIGS. 8C-8D are histograms showing quantifications of peak ΔF/F values or integrated area, before (open bars) vs. after (filled bars) capsaicin injections from 4 different animals (paired t-tests)). The data shown are mean±SEM. FIGS. 8E-8F are results from mice expressing GCaMP3.0 but not the TRPV1 receptor in MrgprB4⁺ neurons, exhibit calcium transients evoked by peripheral injection of α,β-methylene ATP (FIG. 8F), but not with capsaicin in the same field of view (FIG. 8E) (the injections were performed with a 15-20 min. window so as to avoid desensitization from capsaicin). The arrows in FIGS. 8B, 8E and 8F indicate time of stimulus delivery.

FIGS. 9A-9J depict MrgprD⁺ and MrgprB4⁺ fibers activated by mechanical stimuli in multiple ROIs in a given field of view. FIGS. 9A and 9F are schematics illustrating pinching (FIG. 9A) and stroking (FIG. 9F) stimuli. FIGS. 9E and 9J show ROIs used for imaging in FIGS. 9B-9D and 9G-9I, respectively. The rectangles (upper right in FIG. 9E, and lower right in FIG. 9J) are regions used for background subtraction. FIG. 9B shows superimposed traces from different color-coded ROIs (FIG. 9E) in a single trial consisting of 10 pinch stimuli. The light gray bar represents pinching in a specific ipsilateral digit where the stimulus evoked a response, the dark gray bars represent pinching in other ipsilateral digits (see also FIG. 10G-10L), and the black bars represent pinching in contralateral digits (see also FIG. 10A-10F). FIG. 9C depicts the trial average for response to pinching (n=4 trials, 1-4 stimuli/trial), from a single animal. FIG. 9D depicts MPI ΔF/Fₚₑₐₖ calculated from the curves in FIG. 9C. FIG. 9G depicts superimposed traces from different color-coded ROIs (FIG. 9J) in a single trial consisting of 7 brushing stimuli (light gray bars). The onset of the rise in ΔF/F is variably offset from the apparent onset of the stimulus (left edge of gray bars), because the stimulus time stamp pulse is manually actuated (by squeezing the brush between the thumb and forefinger at a specific contact point), and there is some variation in the time elapsed between this actuation and the actual stimulus application to the animal. Alternatively, the receptive field of the activated fiber might lie towards the middle or end of the path of the brush stimulus, and therefore activation would be observed later in the stimulus delivery period. FIG. 9H depicts the trial average for response to brushing (n=5 trials, ˜6 stimuli/trial), from a single animal. FIG. 9I is MPI ΔF/Fₚₑₐₖ calculated from the curves in FIG. 9H. The open and filled bars in FIGS. 9D and 9I are before and after stimulus delivery, respectively. The data in FIGS. 9D and 9I were tested for statistical significance by repeated measures ANOVA, followed by Bonferoni's post-hoc comparisons. The scale bars in FIGS. 9F and 9L are 9 μm and 8.5 μm, respectively. All data shown in FIGS. 9A-9J are mean±SEM.

FIGS. 10A-10L depict regional specificity of MrgprD⁺ fiber activation by pinching stimuli. FIGS. 10A and 10G are schematics illustrating pinching stimuli. FIGS. 10B and 10H are ROIs used for imaging in FIGS. 10C-10I. The rectangles in the lower right of FIGS. 10B and 10H are regions used for background subtraction. FIG. 10C shows superimposed traces from the ROI in FIG. 10B in a single trial consisting of 7 pinch stimuli (the third bar from the left of FIG. 10C represents pinching in a specific ipsilateral digit where pinching evokes a response, the other six bars represent pinching in contralateral digits). FIG. 10D depicts the trial average for response to pinching (n=3 trials, 1 stimulus/trial in a specific ipsilateral digit where pinching evokes a response and 8 stimuli in total in contralateral digits) from a single animal. FIGS. 10E-10F are MPI ΔF/Fₚₑₐₖ (FIG. 10E) or integrated area (FIG. 10F) calculated from the curves in FIG. 10D. FIG. 10I depicts superimposed traces from the ROI in FIG. 10H in a single trial consisting of 8 pinch stimuli (the bar on the most right of FIG. 10J represents pinching in a specific ipsilateral digit where pinching evokes a response, and the other seven bars represent pinching in other ipsilateral digits). FIG. 10J shows the trial average for response to pinching (n=3 trials, 4 stimuli in total in a specific ipsilateral digit where pinching evokes a response and 12 stimuli in total in other ipsilateral digits), from a single animal. FIGS. 10K-10L are MPI ΔF/Fₚₑₐₖ (FIG. 10K) or integrated area (FIG. 10L) calculated from the curves in FIG. 10J. The open and filled bars in FIGS. 10E, 10F, 10K and 10L are before and after stimulus delivery, respectively. The data in 10E, 10F, 10K and 10L were tested for statistical significance by repeated measures ANOVA, followed by Bonferonni's post-hoc comparisons. All data shown are mean±SEM. The scale bars in FIGS. 10B and 10H are 15.6 μm.

FIGS. 11A-11Q depict imaging activity during stroking in tdTomato MrgprB4 fibers. FIG. 11C is a schematic illustrating delivery of brushing stimulus in different zones. FIGS. 11A, 11D, 11G and 11J show visualization, in the same field of view of FIG. 11A GCaMP3.0⁺ (before stimulation), FIG. 11D tdTomato⁺, FIG. 11G superimposed expression of both fluorescent labels and FIG. 11J-F are pseudo-color representations of GCaMP3.0 signal from FIG. 11A during stimulation, in MrgprB4+ central afferent fibers. The insets in FIG. 11G and FIG. 11J are higher magnification views of the boxed region. The scale bar in FIG. 11J is 19.6 μm. White solid line rectangular boxes in FIGS. 11A, 11D, 11G and 11J define region-of-interest (ROI) used for imaging studies in FIGS. 11B, 11E, 11H and 11K, in which GCaMP3.0⁺ and tdTomato⁺ fibers are indistinguishable at this level of resolution (FIG. 11G). ΔF/F responses in the indicated ROI from FIG. 11B G-CaMP3.0, FIG. 11E tdTomato, FIG. 11K, GCaMP3.0 minus tdTomato and FIG. 11H superposition of FIGS. 11B, 11E and 11K to brushing stimuli delivered to hairy skin FIG. 11C. The 4th, 6th, 8th, and 9th bar (counting from left) of FIGS. 11B, 11E, 11H, and 11K, and the 2nd and 4th bar (counting from left) of FIG. 11N represent brushing in ipsilateral zone 1 of FIG. 11C, where stimulation evoked responses, and the other bars in FIGS. 11B, 11E, 11H, 11K, and 11N shows brushing in zones 2, 3, 4 and in the contralateral side where stimulation failed to produce calcium transients in the specific field of view. FIG. 11F shows the average ΔF/F responses to 4 brushing stimuli in zone 1, from a single animal in a single trial. FIGS. 11I and 11L show MPI ΔF/Fₚₑₐₖ (FIG. 11I) or integrated area (FIG. 11L) calculated from the curves in FIG. 11F, respectively. Open and filled bars are 5 frames before and 20 frames after stimulus delivery (vertical dashed line in (FIG. 11F)), respectively. FIG. 11M is F pseudo-colored higher magnification field-of-view of the white dashed rectangular area in FIG. 11J during stimulation. The scale bar is 11.41 μm. FIG. 11N shows GCaMP3.0 Δ/F/F responses to a single trial of brushing stimuli from the ROI in FIG. 11M, which encloses the same fibers as the ROI in FIG. 11J, but imaged at higher magnification. FIG. 11O shows the average responses to 5 brushing stimuli in zone 1 from a single animal in 4 trials, using the ROI in FIG. 11M. FIGS. 11P-11Q show MPI ΔF/Fₚₑₐₖ (FIG. 11P) or integrated area (FIG. 11Q) calculated from the curve in FIG. 11O. Open and filled bars are 5 frames before and 30 frames after stimulus delivery, respectively. All data shown are mean±SEM.

FIGS. 12A-12L depict regional specificity of MrgprB4⁺ fiber activation by brushing stimuli. FIGS. 12A and 12G are schematics illustrating brushing stimuli. In FIG. 12G, a grid is projected onto the mouse delineating separate horizontal and vertical zones. FIGS. 12B and 12H are ROIs used for imaging in FIGS. 12C-12I. Rectangles in the lower right of FIGS. 12B and 12H are regions used for background subtraction. FIG. 12C shows superimposed traces from the ROI in FIG. 12B in a single trial consisting of 12 brushing stimuli (the first seven bars (counting from left) of FIG. 12C represent brushing ipsilaterally that evokes a response, the remaining bars represent contralateral brushing). FIG. 12D shows the trial average for response to brushing (n=4 trials, 28 ipsilateral brushing stimuli and 22 contralateral brushes), from a single animal. FIGS. 12E-12F are MPI ΔF/Fₚₑₐₖ (FIG. 12E) or integrated area (FIG. 12F) calculated from the curves in FIG. 12D. FIG. 12I shows superimposed traces from the ROI in FIG. 12H in a single trial consisting of 14 brushing stimuli (the 1^{st}, 2^{nd}, 5^{th}, 6^{th}, 12^{th}, 13^{th}, and 14^{th} bars (counting from left) of FIG. 12I represent response inducing ipsilateral brushing in horizontal zones 2,3 and the reminding bars represent unresponsive ipsilateral brushing in horizontal zones 1,4). FIG. 12J shows the trial average for response to brushing (n=1 trial, 7 brushing stimuli in responsive zones 2,3 and 7 brushing stimuli in the unresponsive zones 1,4), from a single animal. FIGS. 12K-12L are MPI ΔF/Fₚₑₐₖ (FIG. 12K) or integrated area (FIG. 12L) calculated from the curves in FIG. 12J. Open and filled bars in FIGS. 12E, 12F, 12K, and 12L are before and after stimulus delivery, respectively. The data in FIGS. 12E, 12F, 12K, and 12L were tested for statistical significance by repeated measures ANOVA, followed by Bonferoni's post-hoc comparisons. All data shown are mean±SEM, and the scale bars in FIGS. 12B and 12H are 15 and 19 μm respectively.

FIGS. 13A-13N show imaging activity in MrgprB4⁺ and MrgprD⁺ fibers during alternating, sequential delivery of stroking and pinching stimuli. FIGS. 13A, 13E and 13I are schematics illustrating brushing stimuli and FIGS. 13C, 13G and 13K are schematics illustrating pinching stimuli. FIGS. 13A, 13C and 13E: a grid was projected onto the mouse to delineate a series of horizontal and vertical zones (discussed in further detail herein). FIG. 13M depicts ROIs used for imaging in FIGS. 13B, 13D and 13F. FIG. 13N shows ROIs used for imaging in FIGS. 13H, 13J and 13I. The rectangles in lower right (FIG. 13M) and upper right (FIG. 13N) are regions used for background subtraction. FIGS. 13B, 13D and 13F are sequential trials from the same MrgprB4-tdTomato-2A-Cre/GCaMP3.0 animal. FIG. 13B shows superimposed traces from different ROIs in the same field of view (FIG. 13M), in a single trial consisting of 6 rushing stimuli. FIG. 13D shows superimposed traces from the same ROIs (FIG. 13M) in a consecutive trial, consisting of 5 localized pinching stimuli, in the same zones (identified by the grid) where brushing stimulation evoked responses in FIG. 13B (all bars in FIG. 13D represent pinching stimuli). FIG. 13F depicts superimposed traces from the same ROIs (FIG. 13M) in a consecutive trial consisting of 7 brushing stimuli (all bars in FIG. 13F). FIGS. 13H, 13J, and 13L show sequential trials from the same MrgprD-EGFPCre/GCaMP3 animal. FIG. 13H shows superimposed traces from different ROIs in the same field of view (FIG. 13N), in a single trial consisting of 8 pinching stimuli (the bar on the most left of FIG. 13H represents pinching in a specific ipsilateral digit where pinching evoked a response, the middle six bars of FIG. 13H represent pinching in other ipsilateral digits and the bar on the most right of FIG. 13H represent pinching in a contralateral digit). FIG. 13J shows superimposed traces from the same ROIs (FIG. 13N) in a consecutive trial consisting of 3 brushing stimuli (all bars) in the same digit where pinch stimulation evoked a response in FIG. 13H. FIG. 13K shows superimposed traces from the same ROIs (FIG. 13N) in a consecutive trial consisting of 7 pinch stimuli (the 3^{rd} bar from the left of FIG. 13I represents pinching in a specific ipsilateral digit where pinching evokes a response, the reminding bars of FIG. 13I represent pinching in other contralateral digits). Scale bars in FIGS. 13M and 13N are 8.5 and 9 μm, respectively.

FIGS. 14A-14N depict that MrgprB4⁺ fibers expressing hM3DREADD exhibit calcium transients in response to CNO. FIG. 14E is a schematic illustrating delivery of chemicals to the dorsal spinal cord of MrgprB4-Cre mice co-injected neonatally with Cre-dependent AAV8 viruses encoding CGaMP3.0 and/or hM3DREADD. FIGS. 14A-14D and FIGS. 14I-14L illustrate MrgprB4⁺ central afferents in the same fields of view before (FIGS. 14A, 14B, 14C, 14I, 14J and 14K) and after (FIGS. 14D and 14L) chemical application, from AAV8:GCaMP3.0-injected MrgprB4-Cre mice with (FIG. 14A-14D) or without (FIG. 14I-14L) co-injection of hM3DREADD virus, respectively. FIGS. 14A and 14I, tdTomato; FIGS. 14B and 14J, GCaMP3.0; FIGS. 14C and 14K, merged expression of GCaMP3.0 and tdTomato; FIGS. 14D and 14L, F pseudocolor representation of GCaMP3.0 signal after the addition of CNO and KCL respectively. CNO application produced robust calcium transients in mice co-injected with both GCaMP3.0 and hM3READD viruses (FIG. 14F), whereas no CNO responses were seen in mice injected only with GCaMP3.0 viruses (FIG. 14M). As a positive control, the MrgprB4⁺ fibers in the same field of view (FIG. 14L), used to produce the plot in FIG. 14M showed robust activation after KCl application (FIG. 14N). The arrows in FIGS. 14F, 14M, and 14N indicate time of stimulus delivery. The white rectangles in FIGS. 14A-14C, corresponding to green and pink rectangles in FIG. 14D, indicate Regions-Of-Interest (ROIs) used to produce the pink and green traces in FIG. 14F. White rectangles in FIGS. 14I-14K, corresponding to rectangle in FIG. 14L, indicate ROI used to produce traces in FIGS. 14M and 14N. The boxes on the upper right corner of FIGS. 14D and 14L indicate regions used for background subtraction. FIGS. 14G and 1411 show quantification of peak ΔF/F values (FIG. 14G) or integrated area (FIG. 14H), for the two ROIs (left and right rectangles on the bottom of FIG. 14D before (open bars) vs. after (filled bars) 4 consecutive CNO applications in the spinal cord of the same mouse (each CNO application is followed by washing with imaging solution). Application of imaging solution did not yield any responses. All data shown are mean±SEM.

FIGS. 15A-15F show characterization of the apparatus used for the conditioned place preference. FIG. 15A is a schematic illustrating conditioning apparatus; Chamber A and Chamber B correspond to the histograms shown in FIGS. 15B and 15C, respectively. FIGS. 15B-15D show frequency histograms showing the distribution of times (mean s/min) spent on each of the side chambers of the apparatus (FIGS. 15B and 15C) and in the center chamber (FIG. 15D) during the 30 min pre-test session for all the mice used in the CPP and CPA assays. FIG. 15E shows the mean time (s/min) spent in each chamber of the apparatus by all mice used during the 30 min pre-test. P<0.0001 by repeated measures one way ANOVA followed by Bonferroni post tests. FIG. 15F shows the one way ANOVA (followed by Bonferroni post tests) indicated no significant differences (all P values >0.05) between different groups in mean time spent in Chamber B (FIG. 15C) during the pre-test prior to conditioning (no apparatus bias across groups). There was also no significant difference (all P values >0.05) between groups for those mice for whom Chamber B was the I.N.P. chamber (80% of mice; not shown).

FIGS. 16A-16H show scatter-plot representations of the absolute time (s) spent pre- and post-conditioning for each mouse in the I.N.P. chamber in all groups. Absolute time (sec) spent by each mouse in the I.N.P chamber pre- and post-conditioning with CNO is shown in FIGS. 16A, 16B, 16D, 16E, 16F and 16G, or saline is shown in FIG. 16C. Data points corresponding to the same mouse pre- and post-conditioning are connected by solid black lines. The shaded bars indicate group averages; significant differences (P<0.01, as determined by independently performed paired t tests) between pre- and post-conditioning were found only in the experimental group shown in FIGS. 16A and 16B (MrgprB4-Cre/hM3DREADD mice conditioned with CNO in the I.N.P. chamber). In FIG. 16B, all the mice in FIG. 16A with n=15 were included except for those animals whose pre-test times in the I.N.P chamber were > or <2 standard deviations (P<0.046) from the mean pre-test time of all combined mice. This resulted in exclusion of 3 mice that showed the lowest pre-test time in the I.N.P. chamber as shown in FIG. 16B with n=12. Application of this same procedure to all other control groups resulted in no exclusion of any data points. This procedure should, if anything, bias the data away from showing an effect (by raising the mean pre-test score of the experimental group; compare mean pre-test scores in FIG. 16A vs. FIG. 16B), yet a statistically significant difference was still observed only in the experimental group (FIG. 16B). FIGS. 16C-16G show data from the control groups. FIG. 16C is MrgprB4-Cre/hM3DREADD mice conditioned with saline in both chambers (n=6). FIG. 16D shows results from MrgprD-Cre/hM3DREADD mice conditioned with CNO (n=8). FIGS. 16E and 16F depict MrgprB4-Cre (FIG. 16E, n=9) and MrgprD-Cre (FIG. 16F, n=10) mice injected with a control virus (Cre-dependent AAV8::hrGFP) and conditioned in the I.N.P. chamber with CNO. FIG. 16G shows combined data for all hrGFP-injected control mice n=19 (FIGS. 16E and 16F). FIG. 16H shows that one way ANOVA followed by Bonferroni post tests indicated no significant differences (all P values >0.05) in time spent in I.N.P chamber pre-conditioning between the groups in FIGS. 16B, 16C, 16D, 16E, and 16F. A similar result was obtained using the groups in FIGS. 16A, 16C, 16D, 16E, and 16F (not shown). All data shown are mean±SEM.

FIGS. 17A-17F show results from activation of MrgprB4-Cre/hM3DREADD neurons with CNO induces conditioned place preference. FIGS. 17A-17E show absolute time (sec) spent in each of the 3 indicated chambers for the experimental and control groups before (“pre”) and after (“post”) conditioning with the indicated drug (“train. drug”). FIG. 17A shows the results obtained for MrgprB4-Cre/hM3DREADD mice injected with CNO (n=15); FIG. 17B shows the results obtained for MrgprB4-Cre/hM3DREADD mice conditioned with saline in both chambers (n=6). FIGS. 17C and 17E show the results obtained for MrgprB4-Cre (FIG. 17C, n=9) or MrgprD-Cre (FIG. 17E, n=10) mice injected with a control virus (Cre-dependent AAV8::hrGFP) and conditioned in the I.N.P. chamber with CNO. FIG. 17D shows the results obtained for MrgprD-Cre/hM3DREADD mice injected with CNO and conditioned in the I.N.P (n=8). FIG. 17F shows preference score (=[time in I.N.P chamber]/[(time in I.N.P. chamber)+(time in I.P. chamber)]) for experimental and control groups. In FIGS. 17A-17F, “n.s.” is not significant), *p<0.05, ** p<0.01, and *** p<0.001. All data shown are mean±SEM. Statistical significance was tested by a repeated measures two way mixed ANOVA with group as the between subjects factor and pre/post scores as the within subjects factor. Detection of significant interactions {FIG. 17A, p<0.0001, F(2,42)=22.29; FIG. 17B, no significant interaction; FIG. 17C, p<0.001, F(2,24)=17.08; FIG. 17 D, p<0.01, F(2,21)=6.074; FIG. 17E, p<0.01, F(2,27)=5.630; FIG. 17F, p<0.05, F(4,43)=2.851} and/or main effects {FIG. 17A, group main effect, p<0.0001, F(2,42)=45.05; FIG. 17B, group main effect, p<0.001, F(2,15)=52.48; FIG. 17C, group main effect, p<0.001, F(2,24)=24.82; FIG. 17D, group main effect, p<0.01, F(2,21)=9.209; and FIG. 17E, group main effect, p<0.001, F(2,27)=21.45} FIG. 17F pre/post main effect, p<0.0001, F(1,43)=19.70 was followed by a Bonferoni-corrected post hoc comparison of means. The non-significant trends to an increased preference score for MrgprB4/hrGFP, MrgprD-Cre/hrGFP and MrgprD-Cre/hM3DREADD mice in FIG. 17F reflects a decreased time in the saline-paired (I.P.) compartment, and corresponding increase in the time spent in the neutral compartment (see panels in FIGS. 17C, 17D and 17E), not a statistically significant increase in time spent in the CNO-paired (I.N.P.) compartment (the numerator in the preference score).

FIGS. 18A-18C depict apparatuses used for mechanical stimulation. Images of the touch sensor amplifier box are shown in FIG. 18A and of the probes (paint brush and forceps) in FIG. 18B used for mechanical stimulation during imaging. A circuit diagram of the touch sensor is shown in FIG. 18C.

FIG. 19 show schematic illustration of AAV constructs tested for imaging. The use of CMV promoter and of the loxP-STOP-loxP cassette (rather than the FLEX design for Cre dependence; the bottom 7 constructs) resulted in higher levels of expression in cell bodies and in the central fibers.

FIG. 20 shows ΔF/Fₚₑₐₖ responses in 4 MrgprD mice before and during pinching stimulation. The animals shown are in addition to the animal analyzed in FIG. 2H. Mice 1-3 are the same as those shown in FIG. 2J. Calcium transients were measured for two different ROIs in a given field of view. The data were tested for statistical significance by repeated measures ANOVA, followed by Bonferoni-corrected post hoc comparison of means.

FIG. 21 shows ΔF/Fₚₑₐₖ responses in 12 MrgprB4 mice before and during stroking stimulation. The animals shown are in addition to the animal analyzed in FIG. 3H and FIG. 11. Mice 7, 1 and 12 correspond to mice 1, 2 and 3 respectively as shown in FIG. 3J. Calcium transients were measured for two different ROIs in a given field of view. The data were tested for statistical significance by repeated measures ANOVA, followed by Bonferoni-corrected post hoc comparison of means. ΔF/Fₚₑₐₖ responses in sections 11a and 11b of FIG. 21 refer to measurements in two different fields of view in the same animal.

### DETAILED DESCRIPTION

The description that follows illustrates various embodiments of the subject matter disclosed herein. Those of skill in the art will recognize that there are numerous variations and modifications of the subject matter provided herein that are encompassed by its scope. Accordingly, the description of certain embodiments should not be deemed to limit the scope of the present application.

In addition, in the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the Figures, can be arranged, substituted, combined, and designed in a wide variety of different configurations, all of which are contemplated and make part of this disclosure.

The present application relates to methods for identifying and/or screening for compounds having anxiolytic activities. The method can include, for example, providing a candidate compound; testing the candidate compound for its ability to activate MrgprB4⁺ neurons; and testing the candidate compound for its activity for positive behavioral valence in an animal if the candidate compound activates MrgprB4⁺ neurons. Also disclosed herein are methods for treating anxiety and methods for identifying activating stimuli for sensory neurons, such as MrgprB4⁺ neurons. In some embodiments, the anxiety is caused by itching or pain.

### DEFINITIONS

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, N.Y. 1994); Sambrook et al., Molecular Cloning, A Laboratory Manual, Cold Springs Harbor Press (Cold Springs Harbor, N.Y. 1989). For purposes of the present disclosure, the following terms are defined below.

As used herein, the terms “protein” and “polypeptide” are used interchangeably and refer to a polymer of amino acids. A polypeptide can be of various lengths. Thus, peptides, oligopeptides and proteins are included within the definition of polypeptide. A polypeptide can be with or without N-terminal methionine residues. A polypeptide may include post-translational modifications, for example, glycosylation, acetylation, phosphorylation and the like. Examples of “polypeptide” include, but are not limited to, polypeptides containing one or more analogs of an amino acid (including, for example, unnatural amino acids, non-coded amino acids, etc.), polypeptides with substituted linkages, fusion proteins, as well as polypeptides with other modifications known in the art, both naturally occurring and non-naturally occurring. The term “protein” or “polypeptide” also refer to naturally-occurring allelic variants and proteins that have a slightly different amino acid sequence than those specifically recited above. Allelic variants, though possessing a slightly different amino acid sequence than those recited above, will still have the same or similar biological functions associated with the protein.

Identity or homology with respect to amino acid sequences is defined herein as the percentage of amino acid residues in the candidate sequence that are identical with the known peptides, after aligning the sequences and introducing gaps, if necessary, to achieve the maximum percent homology, and not considering any conservative substitutions as part of the sequence identity. Fusion proteins, or N-terminal, C-terminal or internal extensions, deletions, or insertions into the peptide sequence shall not be construed as affecting homology.

Proteins can be aligned, for example, using CLUSTALW (Thompson et al. Nucleic Acids Res 22:4673-80 (1994)) and homology or identity at the nucleotide or amino acid sequence level may be determined, for example, by BLAST (Basic Local Alignment Search Tool) analysis using the algorithm employed by the programs blastp, blastn, blastx, tblastn and tblastx (Karlin, et al. Proc. Natl. Acad. Sci. USA, 1990, 87:2264-2268 and Altschul, S. F. J. Mol. Evol., 1993, 36:290-300, both of which are herein incorporated by reference in its entirety) which are tailored for sequence similarity searching. The approach used by the BLAST program is to first consider similar segments between a query sequence and a database sequence, then to evaluate the statistical significance of all matches that are identified and finally to summarize only those matches which satisfy a preselected threshold of significance. For a discussion of basic issues in similarity searching of sequence databases, see Altschul et al. (Nature Genetics 6: 119-129 (1994)) which is herein incorporated by reference in its entirety. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff, et al. Proc. Natl. Acad. Sci. USA, 1992, 89:10915-10919, which is herein incorporated by reference in its entirety). For blastn, the scoring matrix is set by the ratios of M (i.e., the reward score for a pair of matching residues) to N (i.e., the penalty score for mismatching residues), wherein the default values for M and N are 5 and −4, respectively. Four blastn parameters were adjusted as follows: Q=10 (gap creation penalty); R=10 (gap extension penalty); wink=1 (generates word hits at every winkth position along the query); and gapw=16 (sets the window width within which gapped alignments are generated). The equivalent Blastp parameter settings were Q=9; R=2; wink=1; and gapw=32. A Bestfit comparison between sequences, available in the GCG package version 10.0, uses DNA parameters GAP=50 (gap creation penalty) and LEN=3 (gap extension penalty) and the equivalent settings in protein comparisons are GAP=8 and LEN=2.

As used herein, the term “variant” refers to a biologically active polypeptide having an ammo acid sequence which differs from the sequence of a native sequence polypeptide disclosed herein, by virtue of an insertion, deletion, modification and/or substitution of one or more amino acid residues within the native sequence. Variants include peptide fragments of at least 5 amino acids, preferably at least 10 amino acids, more preferably at least 15 amino acids, even more preferably at least 20 amino acids that retain a biological activity of the corresponding native sequence polypeptide. Variants also include polypeptides wherein one or more amino acid residues are added at the N- or C-terminus of, or within, a native sequence. Further, variants also include polypeptides where a number of amino acid residues are deleted and optionally substituted by one or more different amino acid residues.

As used herein, the term “conservative variant” refers to alterations in the amino acid sequence that do not adversely affect the biological functions of the protein. A substitution, insertion or deletion is said to adversely affect the protein when the altered sequence prevents or disrupts a biological function associated with the protein. For example, the overall charge, structure or hydrophobic/hydrophilic properties of the protein can be altered without adversely affecting a biological activity. Accordingly, the amino acid sequence can be altered, for example to render the peptide more hydrophobic or hydrophilic, without adversely affecting the biological activities of the protein.

As used herein, the terms “MrgprB4” and “MrgB4” are used interchangeably and refer to a mammalian Mas-related G protein-coupled receptor B4, including but not limited to, murine or human MrgprB4 receptors, MrgprB4 receptor variants, MrgprB4 receptor extracellular domain, and chimeric MrgprB4 receptors. The MrgprB4 can include a protein sequence having at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%, or about 100% sequence identity to a polypeptide described by NCBI Reference Sequence No. NP_{—}991364.1 (SEQ ID NO: 2) or a fragment thereof that has MrgprB4 biological activity. In some embodiments, the MrgprB4 has about 90%, about 91%, about 92%, about 93%, about 94%, about 95%, about 96%, about 97%, about 98%, or about 99%, or about 100% sequence identity to SEQ ID NO: 2.

As used herein, “MrgprB4 nucleic acid molecule” refers to a polynucleotide sequence encoding an MrgprB4 polypeptide.

As used herein, the terms “polynucleotide” and “nucleic acid” are used interchangeably and refer to polymeric forms of nucleotides of any length. Thus, oligonucleotides are included within the definition of polynucleotide. “Nucleic acid” can be RNA or DNA that encodes a protein or peptide as defined above, is complementary to a nucleic acid sequence encoding such peptides, hybridizes to such a nucleic acid and remains stably bound to it under appropriate stringency conditions, exhibits at least about 50%, 60%, 70%, 75%, 85%, 90% or 95% nucleotide sequence identity across the open reading frame, or encodes a polypeptide sharing at least about 50%, 60%, 70% or 75% sequence identity, preferably at least about 80%, and more preferably at least about 85%, and even more preferably at least about 90 or 95% or more identity with the peptide sequences. Specifically contemplated are genomic DNA, cDNA, mRNA and antisense molecules, as well as nucleic acids based on alternative backbones or including alternative bases whether derived from natural sources or synthesized. Such hybridizing or complementary nucleic acids, however, are defined further as being novel and unobvious over any prior art nucleic acid including that which encodes, hybridizes under appropriate stringency conditions, or is complementary to nucleic acid encoding a protein according to the present invention.

As used herein, the terms nucleic acid, polynucleotide and nucleotide are interchangeable and refer to any nucleic acid, whether composed of phosphodiester linkages or modified linkages such as phosphotriester, phosphoramidate, siloxane, carbonate, carboxymethyl ester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sultone linkages, and combinations of such linkages.

The terms nucleic acid, polynucleotide and nucleotide also specifically include nucleic acids composed of bases other than the five biologically occurring bases (adenine, guanine, thymine, cytosine and uracil). For example, a polynucleotide of the invention might contain at least one modified base moiety which is selected from the group including but not limited to 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyl-uracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5N-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and 2,6:-diaminopurine.

Furthermore, a polynucleotide may comprise at least one modified sugar moiety selected from the group including but not limited to arabinose, 2-fluoroarabinose, xylulose, and hexose.

As used herein, a nucleic acid molecule is said to be “isolated” when the nucleic acid molecule is substantially separated from contaminant nucleic acid molecules encoding other polypeptides.

Highly related gene homologs are polynucleotides encoding proteins that have at least about 60% amino acid sequence identity with the amino acid sequence of a naturally occurring native sequence polynucleotide disclosed herein, preferably at least about 65%, 70%, 75%, 80%, with increasing preference of at least about 85% to at least about 99% amino acid sequence identity, in 1% increments.

As used herein, a “subject” refers to an animal that is the object of treatment, observation or experiment. “Animal” includes cold- and warm-blooded vertebrates and invertebrates such as fish, shellfish, reptiles, and in particular, mammals. “Mammal,” as used herein, refers to an individual belonging to the class Mammalia and includes, but not limited to, humans, domestic and farm animals, zoo animals, sports and pet animals. Non-limiting examples of mammals include mice; rats; rabbits; guinea pigs; dogs; cats; sheep; goats; cows; horses; primates, such as monkeys, chimpanzees and apes, and, in particular, humans. In some embodiments, the mammal is a human. However, in some embodiments, the mammal is not a human.

As used herein, a compound having an “anxiolytic activity” refers to any compound (e.g., small molecule (for example, an organic or inorganic molecule), peptides, peptide mimetics, proteins, nucleic acids, and antibodies) that can cause a positive and/or desired effect on the subject. Compound having an “anxiolytic activity” are also referred to as anxiolytic compounds herein. For example, anxiolytic compounds may positively impact the mood of a subject; prevent, reduce or stop anxiety in a subject; prevent, relieve or stop stress in a subject; cause pleasurable effect on a subject; induce pleasant feeling of the subject; and/or prevent, relieve or stop one or more undesired sensations (e.g., itching and pain) in a subject. In some embodiments, the anxiety or stress is caused by itching or pain. In some embodiments, administration of an anxiolytic compound to a subject can induce or enhance positive-valence behavior in the subject. In some embodiments, administration of an anxiolytic compound to a subject can make the subject relax and/or feel comfortable. In some embodiments, administration of an anxiolytic compound to a subject makes the subject relax and/or feel comfortable. In some embodiments, administration of an anxiolytic compound to a subject prevents, relieves, or stops discomfort in the subject. In some embodiments, administration of an anxiolytic compound to a subject prevents, relieves, or stops itching or pain in the subject.

As used herein, the term “transfection” refers to the introduction of a nucleic acid into a host cell, such as by contacting the cell with a recombinant AAV virus as described below.

The term “construct,” as used herein, refers to a recombinant nucleic acid that has been generated for the purpose of the expression of a specific nucleotide sequence(s), or that is to be used in the construction of other recombinant nucleotide sequences.

As used herein, the term “agonist” is used in the broadest sense and refers to any molecule or compound that fully or partially activates, stimulates, enhances, or promotes one or more of the biological properties of a polypeptide disclosed herein. Agonists may include, but are not limited to, small organic and inorganic molecules, nucleic acids, peptides, peptide mimetics and antibodies.

As used herein, the term “biological property” or “biological activity” refers to a biological function caused by a protein, such as an Mrgpr (including, but not limited to, MrgprB4), an agonist of an Mrgpr (including, but not limited to MrgprB4 agonists and MrgprB4 agonists), or other compound disclosed herein. Biological properties of Mrgprs include, but are not limited to, G-protein coupled receptor signal transduction activity, regulating the function or development of noceptive neurons, functioning as itch receptors, modulating opioid signaling, and regulating calcium-signaling pathway. With regard to agonists of Mrgprs, biological activity refers, in part, to the ability to fully or partially activate, stimulate, enhance, or promote the biological properties of Mrgprs. For example, an MrgprB4 agonist can have the ability to stimulate, enhance, or promote the activation of MrgprB4. Preferred biologic activities of agonists of Mrgprs include, but are not limited to, treating, alleviating, preventing or stopping anxiety; treating, alleviating, preventing or stopping unpleasant sensations such as itching and pain; treating, alleviating, preventing stress; and inducing or enhancing positive-valence behavior in a subject.

As used herein, the term “treatment” refers to a clinical intervention made in response to a disease, disorder or physiological condition manifested by a patient, for example anxiety, stress, itching and pain. The aim of treatment may include, but is not limited to, one or more of the alleviation or prevention of symptoms, slowing or stopping the progression or worsening of a disease, disorder, or condition and the remission of the disease, disorder or condition. In some embodiments, “treatment” refers to both therapeutic treatment and prophylactic or preventative measures. Those in need of treatment include those already affected by a disease or disorder or undesired physiological condition as well as those in which the disease or disorder or undesired physiological condition is to be prevented. For example, in some embodiments treatment may alleviate anxiety, including anxiety resulting from an existing condition or disorder, or to prevent anxiety in situations where anxiety is likely to be experienced. As another example, in some embodiments, treatment may alleviate, prevent, slow, or stop anxiety. In some embodiments treatment may alleviate itching or pain, including itching or pain resulting from an existing condition or disorder, or to prevent itching or pain in situations where itching or pain is likely to be experienced. As another example, in some embodiments, treatment may alleviate, prevent, slow, or stop itching or pain.

As used herein, the term “effective amount” or “effective dose” refers to an amount sufficient to effect beneficial or desirable clinical results. An effective amount of an agonist is an amount that is effective to treat a disease, disorder or unwanted physiological condition. For example, in the case of anxiety, stress, itching or pain, the effective amount of an activator of MrgprB4⁺ neurons (e.g., an MrgprB4 agonist) is sufficient to treat, prevent, alleviate or stop anxiety, stress, itching or pain in the subject. The effective dose can be a single dose, or can comprise multiple doses given over a period of time. In some embodiments, the amount used can be sufficient to activate MrgprB4 in the cell, tissue and/or the organism.

“Pharmaceutically acceptable” carriers, excipients, or stabilizers are ones which are nontoxic to the cell or mammal being exposed thereto at the dosages and concentrations employed. Often the physiologically acceptable carrier is an aqueous pH buffered solution such as phosphate buffer or citrate buffer. The physiologically acceptable carrier may also comprise one or more of the following: antioxidants including ascorbic acid, low molecular weight (less than about 10 residues) polypeptides, proteins, such as serum albumin, gelatin, immunoglobulins; hydrophilic polymers such as polyvinylpyrrolidone, amino acids, carbohydrates including glucose, mannose, or dextrins, chelating agents such as EDTA, sugar alcohols such as mannitol or sorbitol, salt-forming counterions such as sodium, and nonionic surfactants such as Tween™, polyethylene glycol (PEG), and Pluronics™.

As used herein, the term “topical composition” refers to a composition that can be topically applied to mammalian keratinous tissue. The term “cosmetic composition” as used herein to refer to topical cosmetic compositions as defined under the heading “Kosmetika” in Rump Lexikon Chemie, 10th edition 1997, Georg Thieme Verlag Stuttgart, New York. In some embodiments, a topical composition comprising an activator of MrgprB4⁺ neurons is a topical cosmetic composition. In some embodiments, a topical composition including one or more activators of MrgprB4 can also contain adjuvants and additives typically used in topical formulations, such as preservatives/antioxidants, fatty substances or oils, water, organic solvents, silicones, thickeners, softeners, emulsifiers, sunscreens, antifoaming agents, moisturizers, aesthetic components such as fragrances, surfactants, fillers, sequestering agents, anionic, cationic, nonionic or amphoteric polymers or mixtures thereof, propellants, acidifying or basifying agents, dyes, colorings/colorants, abrasives, absorbents, essential oils, skin sensates, astringents, pigments or nanopigments, or any other ingredients usually formulated into cosmetic compositions. Examples of ingredients commonly used in the topical formulation (e.g., skin care industry) that are suitable for use in the compositions disclosed herein are described in the CTFA Cosmetic Ingredient Handbook, Second Edition (1992) without being limited thereto.

### MrgprB4⁺ Neurons

Mrgprs (Mas-Related G-Protein-Coupled Receptors, also called sensory neuron specific receptors (SNSRs)) are a large family of orphan G-protein-coupled receptors (GPCRs). The Mrgpr gene family contains more than 50 members in the mouse genome, which can be grouped into several subfamilies: MrgprA1-22, MrgprB1-13, MrgprC1-14, and MrgprD-G (Dong et al., Cell 106:619-632, 2001; Zylka et al., Proc. Natl. Acad. Sci. USA 100:10043-10048, 2003). The Mrgpr family is smaller in other species such as rat and human, suggesting an atypical expansion of Mrgpr genes in mice (Dong et al., 2001; Zylka et al., 2003). Mrgprs are specifically expressed in subsets of small-diameter sensory neurons. For example, the MrgprA and MrgprD genes were specifically expressed in a subset of DRG sensory neurons (US Patent Publication No. 20030092035, the content of which is hereby expressly incorporated by reference in its entirety). In contrast, MrgprB1-5 were not detectably expressed in the dorsal root ganglia (DRG). Expression of MrgprB1 and MrgprB2 has been observed in scattered cells in the epidermal layer of skin in newborn mice, as well as in the spleen and the submandibular gland (US Patent Publication No. 20030092035). These cells appear to be immune cells that play a role in wound repair. In contrast, MrgprB3, MrgprB4 and MrgprB5 do not appear to be expressed in any of these tissues in mice.

MrgprB4⁺ neurons are a rare population of unmyelinated, nonpeptidergic sensory neurons that have been characterized morphologically in Liu et al., Nature Neuroscience, 10(8):946-948. MrgprB4⁺ neurons form sensory fibers that exclusively innervate hairy skin. These MrgprB4⁺ fibers terminate in large arborizations similar in size and distribution to C-fiber tactile afferent receptive fields. As disclosed herein, unlike other molecularly defined mechano-sensory C-fibre subtypes, MrgprB4⁺ neurons could not be detectably activated by sensory stimulation of the skin ex vivo.

Stroking of the skin produces pleasant sensation that can occur during social interactions with conspecifics, such as grooming. As disclosed herein, MrgprB4⁺ neurons are activated by massage-like stroking of hairy skin, but not by noxious punctate mechanical stimulation. In addition, pharmacogenetic activation of Mrgprb4-expressing neurons in freely behaving mammals promoted conditioned place preference, indicating that such activation is positively reinforcing and/or anxiolytic. In some embodiments, activation of MrgprB4⁺ neurons prevents, reduces, or stops anxiety. In some embodiments, MrgprB4⁺ neurons are used to identify compounds having anxiolytic activities. In some embodiments, the compound having anxiolytic activity is an MrgprB4⁺ agonist. Agonists of MrgprB4 receptor can activate MrgprB4 receptors in nociceptive neurons, and thus be used to treat a subject suffering from anxiety. Moreover, in some embodiments, prevention, inhibition or alleviation of anxiety is achieved by using an agonist of MrgprB4 receptor. In some embodiments, the anxiety or stress is caused by itching or pain. Activation of MrgprB4⁺ neurons can also prevent, reduce, or stop an unpleasant sensation. Agonists of MrgprB4 receptor can activate MrgprB4 receptors in nociceptive neurons, and thus be used to prevent, reduce, or stop an unpleasant sensation (e.g., itching or pain) in a subject. Moreover, in some embodiments, prevention, inhibition or alleviation of the unpleasant sensation is achieved by using an agonist of MrgprB4 receptor. Without being bound by any particular theory, it is believed that activation of MrgprB⁺ neurons can cause a positive and/or desired effect on a subject having the MrgprB⁺ neurons. For example, activation of MrgprB⁺ neurons may positively impact the mood of the subject; prevent, reduce or stop anxiety in the subject; prevent, relieve or stop stress in the subject; cause pleasurable effect on the subject; induce or enhance pleasant feeling of the subject; and/or prevent, relieve or stop one or more undesired sensations (e.g., itching and pain) in the subject. In some embodiments, activation of MrgprB⁺ neurons in a subject can lead to positive-valence behavior in the subject. In some embodiments, activation of MrgprB⁺ neurons in a subject makes the subject relax and/or feel comfortable. In some embodiments, activation of MrgprB⁺ neurons in a subject prevents, relieves, or stops discomfort in the subject. In some embodiments, activation of MrgprB⁺ neurons in a subject prevents, relieves, or stops itching or pain in the subject. In some embodiments, activation of MrgprB⁺ neurons in a subject induces or enhances positive-valence behavior in the subject. In some embodiments, activation of MrgprB⁺ neurons in a subject make the subject relax and/or feel comfortable.

### Activators of MrgprB4⁺ Neurons

As used herein, an activator of MrgprB4⁺ neurons is a molecule that can partially or fully activate a biological activity of MrgprB4⁺ neurons. Examples of the biological activity of MrgprB4⁺ neurons includes, but is not limited by, detection of skin-to-skin contact (for example, the contact between individuals that is associated with affiliative emotional behaviors), caress-like contact, massaging, stroking, pinching, brushing, and/or grooming.

Various types of activators of MrgprB4⁺ neurons can be identified as anxiolytic compounds and used in the methods disclosed herein for, for example, treating anxiety, itching or pain; preventing, reducing, and stopping stress, causing pleasurable effect on the subject; inducing or enhancing pleasant feelings of the subject. For example, the activators of MrgprB4 can be small molecules (for example, an organic or inorganic molecule), peptides, peptide mimetics, proteins, nucleic acids, and antibodies. In some embodiments, the activator of MrgprB4⁺ neurons is ATP, for example α,β-methylene (Me) ATP. In some embodiments, the activator of MrgprB4⁺ neurons is clozapine-N-oxide. The mechanism by which the activator is able to activate MrgprB4⁺ neurons can also vary. For example, the activator may bind to one or more receptors on the surface of MrgprB4⁺ neurons, including MrgprB4 receptors, to activate the MrgprB4⁺ neurons. In other embodiments the activator may act indirectly. For example, the activator may interact with a compound that inhibits one or more biological activities of MrgprB4⁺ neurons (that is, an inhibitor of MrgprB4⁺ neurons) to neutralize the inhibitory effect of the compound, and thus to activate MrgprB4⁺ neurons. As yet another example, the activator may bind to a cell that can trigger the activation of the MrgprB4⁺ neurons.

### Agonists of MrgprB4

As discussed above, the term “agonist” is used herein in a broad sense and includes any molecule that partially or fully activates a biological activity mediated by one or more Mrgprs, such as MrgprB4. The term “agonist” also includes any molecule that mimics a biological activity mediated by an Mrgpr, such as MrgprB4, and molecules that specifically change, preferably increase, the function or expression of the Mrgpr, or the efficiency of signaling through the Mrgpr.

In some embodiments, agonists of Mrgprs, for example MrgprB4 agonists, can be used to screen for compounds having anxiolytic activities. Preferably such agonists are also screened to identify those agonists that are activators for neuron expressing Mrgpr receptors (e.g., MrgprB4⁺ neurons). Such agonists of Mrgpr (e.g., agonists of MrgprB4) can be used to stimulate, enhance, or promote one or more of the biological properties of Mrgpr (e.g., MrgprB4). In some embodiments, agonists of Mrgpr (e.g., agonists of MrgprB4) can be used to directly activate Mrgpr receptors (e.g., MrgprB4 receptors). In some embodiments, agonists of an Mrgpr receptor (e.g., MrgprB4) can be used to positively allosterically modulate the Mrgpr receptor (e.g., MrgprB4) or another Mrgpr. In other words, the agonist of the Mrgpr receptor (e.g., MrgprB4) may be able to interact with one or more Mrgprs to increase the Mrgpr activation triggered by another Mrgpr agonist or Mrgpr binding partner. In some embodiments, an agonist of Mrgpr can bind to the MrgprB4's allosteric site and enhance the ability of an Mrgpr agonist to activate one or more biological properties of the Mrgpr. The Mrgpr is, in some embodiments, MrgprB4.

The biological activity mediated by MrgprB4 may be activated by an agonist in any of a variety of ways. In some embodiments, an MrgprB4 agonist can act directly on MrgprB4 receptor (for example, by binding to the MrgprB4 receptor) and trigger the receptor activity of MrgprB4. Non-limiting examples of such Mrgpr agonists include ATP and clozapine-N-oxide. In some embodiments, an MrgprB4 agonist can enhance the ability of MrgprB4 to interact with a ligand of MrgprB4 receptor. In some embodiments, a first MrgprB4 agonist can enhance the activation of MrgprB4 by a second MrgprB4 agonist.

In some embodiments, the MrgprB4 agonist can be a constitutively active mutant MrgprB4, for example a constitutively active mutant MrgprB4. In some embodiments, an MrgprB4 agonist can modulate the level of MrgprB4 gene expression, for example increasing the level of transcription of the MrgprB4 gene. In some embodiments, an MrgprB4 agonist can modulate the levels of MrgprB4 protein, in cells, tissues or the body of a subject by, for example, increasing the translation of MrgprB4 mRNA, or decreasing the degradation of MrgprB4 mRNA or MrgprB4 protein.

In some embodiments, the MrgprB4 agonist interacts with MrgprB4 directly and triggers the activation of MrgprB4. The MrgprB4 agonist can also, for example, enhance the interaction of MrgprB4 with a binding partner or ligand, enhance MrgprB4 gene expression, increase the number of MrgprB4 receptors on the cell surface, and/or modulate the level of MrgprB4 protein in the cell, tissue or body of a subject. In some embodiments, the MrgprB4 agonist may interact with a compound that is in an MrgprB4 dependent pathway, for example, upstream or downstream from MrgprB4. In still other embodiments, the MrgprB4 agonist may bind to MrgprB4 to enhance the activation of MrgprB4 triggered by a second MrgprB4 agonist.

In some embodiments, the MrgprB4 agonist can be a positive allosteric modulator of a second MrgprB4 agonist. In some embodiments, the MrgprB4 agonist can enhance the gene expression or the level of a second MrgprB4 agonist in the body of a subject.

The types of MrgprB4 agonists are not limited in any way. Non-limiting examples of MrgprB4 agonists include small molecules (including both organic and inorganic molecules), peptides, peptide mimetics, proteins, nucleic acids, and antibodies. In some embodiments, the MrgprB4 agonist is a small molecule that binds to MrgprB4. In some embodiments, the MrgprB4 agonist can be a peptide. In some embodiments, the MrgprB4 agonist is ATP, for example α,β-methylene (Me) ATP. In some embodiments, the MrgprB4 agonist is clozapine-N-oxide.

### Identification of Activators of MrgprB4⁺ Neurons

In some embodiments, identification of activators of MrgprB4⁺ neurons comprises screening compounds for their ability to act as MrgprB4 agonists. After identification, MrgprB4 agonists can be screened for their ability to activate MrgprB4⁺ neurons. In some embodiments, compounds are also screened to determine whether or not they activate MrgprB4 agonist. Screening assays are well known in the art and can readily be adapted to identify agonists of Mrgprs, such as MrgprB4. As discussed above, agonists of Mrgprs may include compounds that interact with (e.g., bind to) an Mrgpr; compounds that enhance the interaction of an Mrgpr with its binding partner, cognate or ligand (e.g., a positive allosteric modulator of an Mrgpr ligand or an Mrgpr agonist); and compounds that modulate, preferably increase, the level of Mrgpr in the cell, tissue or body of a subject, such as compounds that modulate Mrgpr gene expression. Assays may additionally be utilized to identify compounds that bind to Mrgpr gene regulatory sequences (e.g., promoter sequences) and, consequently, may modulate Mrgpr gene expression.

The compounds which may be screened include, but are not limited to small molecules (including both organic and inorganic molecules), peptides, proteins, antibodies and fragments thereof, and other organic compounds (e.g., peptidomimetics). The compounds can include, but are not limited to, soluble peptides, including members of random peptide libraries (see e.g., Lam, K. S. et al., 1991, Nature 354:82-84; Houghten, R. et al., 1991, Nature 354:84-86), and combinatorial chemistry-derived molecular libraries made of D- and/or L-configuration amino acids, phosphopeptides (including, but not limited to members of random or partially degenerate, directed phosphopeptide libraries; see e.g., Songyang, Z. et al., 1993, Cell 72:767-778), antibodies (including, but not limited to, polyclonal, monoclonal, humanized, anti-idiotypic, chimeric or single chain antibodies, and FAb, F(abN)₂ and FAb expression library fragments, and epitope-binding fragments thereof), and small organic or inorganic molecules, including libraries thereof. Other compounds that can be screened in accordance with the present application include, but are not limited to, small organic molecules, for example, those that are able to cross the blood-brain barrier.

Many methods are available for generating random or directed synthesis (e.g., semi-synthesis or total synthesis) of any number of polypeptides, chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, chemical compounds to be used as candidate compounds can be synthesized from readily available starting materials using standard synthetic techniques and methodologies known to those of ordinary skill in the art. Synthetic chemistry transformations and protecting group methodologies (protection and deprotection) useful in synthesizing the compounds identified by the methods described herein are known in the art and include, for example, those such as described in R. Larock, Comprehensive Organic Transformations, VCH Publishers (1989); T. W. Greene and P. G. M. Wuts, Protective Groups in Organic Synthesis, 2nd ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

Libraries of known compounds, including natural products or synthetic chemicals, and biologically active materials, including proteins, can be screened for compounds which can activate Mrgprs, including MrgprB4.

Small molecules can also have the ability to activate Mrgprs (including MrgprB4) and thus may be screened for such activity. In some embodiments, small molecules can have a molecular weight of less than about 10 kD, about 8 kD, about 5 kD, and about 2 kD. Such small molecules may include naturally-occurring small molecules, synthetic organic or inorganic compounds, peptides and peptide mimetics. However, small molecules in the present application are not limited to these forms. Extensive libraries of small molecules are commercially available and a wide variety of assays are well known in the art to screen these molecules for the desired activity.

In some embodiments, agonists of Mrgprs (e.g., compounds that specifically bind and activate an Mrgpr polypeptide, such as an MrgprB4 agonist) are identified from large libraries of natural product or synthetic (or semisynthetic) extracts or chemical libraries or from polypeptide or nucleic acid libraries, according to methods known in the art. Those skilled in the field of drug discovery and development will understand that the precise source of test extracts or compounds is not critical to the screening procedure(s) disclosed herein. Agents used in screens may include those known as therapeutics for the treatment of conditions such as anxiety, stress, itching, and/or pain. Virtually any number of unknown chemical extracts or compounds can be screened using the methods described herein. Examples of such extracts or compounds include, but are not limited to, plant-, fungal-, prokaryotic- or animal-based extracts, fermentation broths, and synthetic compounds, as well as the modification of existing polypeptides.

Libraries of natural polypeptides in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). Such polypeptides can be modified to include a protein transduction domain using methods known in the art and described herein. In addition, natural and synthetically produced libraries are produced, if desired, according to methods known in the art, e.g., by standard extraction and fractionation methods. Examples of methods for the synthesis of molecular libraries can be found in the art, for example in: DeWitt et al, Proc. Natl. Acad. Set U.S.A. 90:6909, 1993; Erb et al, Proc. Natl. Acad. Sci. USA 91:11422, 1994; Zuckermann et al, J. Med. Chem. 37:2678, 1994; Cho et al, Science 261:1303, 1993; Carrell et al, Angew. Chem. Int. Ed. Engl. 33:2059, 1994; Carell et al, Angew. Chem. Int. Ed. Engl. 33:2061, 1994; and Gallop et al, J. Med. Chem. 37:1233, 1994. Furthermore, if desired, any library or compound is readily modified using standard chemical, physical, or biochemical methods.

In some embodiments, candidate agonist compounds can be identified by first identifying those that specifically bind to MrgprB4 polypeptide and subsequently testing their effect on MrgprB4 biological activity (e.g., using Ca²⁺ influx). The interaction of a compound with MrgprB4 polypeptide can be readily assayed using any number of standard binding techniques and functional assays well known in the art.

In some embodiments, a candidate compound that binds to an MrgprB4 polypeptide may be identified using a chromatography-based technique. For example, an MrgprB4 polypeptide may be purified by standard techniques from cells engineered to express the polypeptide, or may be chemically synthesized, once purified the peptide is immobilized on a column. A solution of candidate compound is then passed through the column, and a compound that specifically binds the MrgprB4 polypeptide or a fragment thereof can be identified on the basis of its ability to bind to MrgprB4 polypeptide and to be immobilized on the column. To isolate the compound, the column can be washed to remove non-specifically bound molecules, and the agent of interest is then released from the column and collected. The compound isolated by this method (or any other appropriate method) may, if desired, be further purified (e.g., by high performance liquid chromatography). In addition, these candidate compounds may be tested for their ability to modulate MrgprB4 (e.g., as described herein).

A candidate compound, such as a compound that specifically binds to MrgprB4, can be tested for activity in an in vitro assay or in vivo assay for its ability to activate MrgprB4 receptor, and subsequently for its ability to activate MrgprB4⁺ neurons. For example, a candidate compound may be tested in vitro for interaction and binding with an MrgprB4 polypeptide and then for its ability to modulate MrgprB4 activity. A variety of methods well known in the art can be used to measure the ability of a molecule to activate an Mrgpr receptor, such as MrgprB4. The ability to modulate MrgprB4 activity may be assayed in vitro by any standard assay for G-protein coupled receptor activity, such as Ca²⁺ influx assay, or by patch clamp or other assay for electrical activity. In some embodiments, the test compounds can be screened using HEK293 cells stably transfected with human MrgprB4 in an intracellular calcium mobilization assay with the fluorometric imaging plate reader (FLIPR, Molecular Devices) as described by Sulivan et al (J. Mol. Biol. 1993, 234:779-815).

In some embodiments, the level of MrgprB4 activation by a potential MrgprB4 agonist can be measured by methods described in Wroblowski et al. (J. Med. Chem., 2009, 52:818-825). As another example, an agonist of Mrgprs (such as an MrgprB4 agonist) can be identified using gene reporter assay and function receptor assay. In an example, a function receptor assay called Receptor Selection and Amplification Technology (R-SAT) can be used to detect the extent a test compound can activate Mrgpr and compare the activation of Mrgpr achieved by the test compound with that achieved by a known MrgprB4 agonist. R-SAT is described in detail in U.S. Pat. Nos. 5,707,798; 5,912,132; and 5,955,281, each of which is incorporated by reference herein in its entirety.

One skilled in the art will appreciate that the effects of a candidate compound on a cell is typically compared to a corresponding control cell not contacted with the candidate compound. The screening methods include, but are not limited to, comparing Ca²⁺ influx in an MrgprB4-expressing cell contacted by a candidate agent with Ca²⁺ influx in an untreated control cell. In some embodiments, the expression or activity of MrgprB4 in a cell treated with a candidate compound is compared to untreated control samples to identify a candidate compound that increases the expression or activity of MrgprB4 in the contacted cell. Polypeptide or polynucleotide expression can be compared by procedures well known in the art, such as Western blotting, flow cytometry, immunocytochemistry, binding to magnetic and/or MrgprB4-specific antibody-coated beads, in situ hybridization, fluorescence in situ hybridization (FISH), ELISA, microarray analysis, RT-PCR, Northern blotting, or colorimetric assays, such as the Bradford Assay and Lowry Assay.

In some embodiments, identification of activators of MrgprB4⁺ neurons is carried out in vitro or ex vivo. For example, a candidate compound can be added to a skin-nerve culture containing MrgprB4⁺ neurons for detecting activation of MrgprB4⁺ neurons. For example, electrophysiological analysis of MrgprB4⁺ neurons in cultures can be performed to detect the extent of responses (or lack of response) of MrgprB4⁺ neurons to the chemical stimuli resulted from the candidate compound. In some embodiments, identification of activator of MrgprB4⁺ neurons is carried out in vivo, for example in intact subjects (e.g., mammals). The administration of the candidate compound to the subject can be carried out via various routes. For example, the candidate compound can be administered to the animal via injection, including but not limited to injection into spinal cord of the subject and peripheral injection into the skin of the subject. Various methods can be used to detect activation of MrgprB4⁺ neurons in vivo. For example, calcium imaging (e.g., two-proton imaging through a spinal cord laminectomy) can be performed on the subject to detect activation of MrgprB4⁺ neurons.

### Testing Candidate Compounds for Anxiolytic Activity

Once identified or provided, an activator for MrgprB4⁺ neurons (e.g., an MrgprB4 agonist), can be further tested for its ability to stimulate and/or enhance positive valence behavior. As used herein, the term “positive valence behavior” refers to a non-verbal transmission of a behavioral feeling or emotion from one subject to another and received in a positive manner. For example, when positive valence behavior occurs, the receiver perceives the positive transmission and may return positive gesture to the initiator, show desire to return to the location where the non-verbal transmission occurs, and/or welcome further contact. The form of non-verbal transmission can vary. For example, the non-verbal transmission can be skin-to-skin contact (e.g., touching), or contact that involves skin (e.g., pinching, massaging, stroking, brushing, and grooming). Compounds that are found to stimulate positive valence behavior may be used to stop, reduce or prevent anxiety in a subject suffering from anxiety. In some embodiments, the anxiety is caused by itching or pain.

Once identified or provided, an activator for MrgprB4⁺ neurons (e.g., an MrgprB4 agonist), can also be further tested for its ability to positively impact the mood of the subject; for its ability to prevent, reduce or stop anxiety in the subject; for its ability to prevent, relieve or stop stress in the subject; for its ability to cause pleasurable effect on the subject; for its ability to induce or enhance pleasant feeling of the subject; and/or for its ability to prevent, relieve or stop one or more undesired sensations (e.g., itching and pain) in the subject. An activator for MrgprB4⁺ neurons (e.g., an MrgprB4 agonist) can also be further tested for its ability to make a subject relax and/or feel comfortable; for its ability to prevent, relieve, or stop discomfort in the subject. In some embodiments, the activator for MrgprB4⁺ neurons (e.g., an MrgprB4 agonist) is further tested for its ability to prevent, relieve, or stop itching or pain in the subject.

Various methods can be used to determine if a compound can stimulate positive valence behavior of a subject. For example, a conditioned place preference assay (CPP assay) can be used to detect positive valence behavior of a subject. CPP assay is a form of Pavlovian conditioning used to measure the motivational effects of objects or experiences. CPP assay is described in for example Tzschentke, Addict. Biol. 12:227-462 (2007) and Panksepp & Lahvis, Genes Brain Behav. 6:661-671 (2007). This method can also be used to measure conditioned place aversion (CPA) with an identical procedure involving aversive stimuli instead. CPP procedure has been used to measure extinction and reinstatement of the conditioned stimulus. For example, certain drugs are used in this paradigm to measure their reinforcing properties. The CPP assay can be biased or unbiased. The biased CPP assay allows the subject to explore the apparatus, and the compartment they least prefer is the one that the drug is administered in and the one they most prefer is the one where the vehicle is injected. This method allows the subject to choose the compartment they get the drug and vehicle in. In comparison, the unbiased CPP assay does not allow the animal to choose what compartment they get the drug and vehicle in and instead the researcher chooses the compartments. In some embodiments, an unbiased CPP assay is used to determine the ability of a compound to stimulate positive valence behavior. In some embodiments, a biased CPP assay is used to determine the ability of a compound to stimulate positive valence behavior. In some embodiments, the CPP assay comprises determining conditioned place aversion.

The route by which the candidate compound is administered to the subject for determining its ability to stimulate positive valence behavior is not particularly limited. For example, the test compound can be administered to the subject via injection or a topical application. In some embodiments, a candidate compound is directly administered to the spinal cord of a subject. In some embodiments, the test compound is applied to the subject peripherally, including but not limited to peripheral injection. In some embodiments, the test compound is applied to the subject topically. The test compound may be in a pharmaceutical or cosmetic composition. In some embodiments, the test compound is in a topical composition, for example a topical cosmetic composition. Non-limiting examples of topical formulation include lotion, cream, foam, ointment, gel, transdermal patch, powder, and spray.

As disclosed herein, known MrgprB4 agonists may be used for treating anxiety, stress, or an unpleasant sensation (e.g., itching and pain) in a subject. In some embodiments, the known MrgprB4 agonists are used in the method for identifying compounds having anxiolytic activity.

In some embodiments, candidate compounds that stimulate positive valence behavior in the subject are selected. In other embodiments, compounds that do not stimulate positive valence behavior in the subject are eliminated from consideration as therapeutic agents for the treatment of anxiety, stress, or an unpleasant sensation (e.g., itching and pain). In some embodiments, MrgprB4 agonists are tested for their ability to stimulate positive valence behavior in two or more animal models.

Compounds identified as a compound capable of stimulating and/or enhancing positive valence behavior may be used, for example, as therapeutics to treat or prevent the onset of a disease or disorder characterized by anxiety or an unpleasant sensation (e.g., itching and pain). In addition, the compounds may also be used to positively impact the mood of a subject; prevent, reduce or stop anxiety in a subject; prevent, relieve or stop stress in a subject; cause pleasurable effect on a subject; induce pleasant feeling of the subject; and/or prevent, relieve or stop one or more undesired sensations (e.g., itching and pain) in a subject. In some embodiments, the compounds make the subject relax and/or feel comfortable. In some embodiments, the compounds make the subject relax and/or feel comfortable. In some embodiments, the compounds prevent, relieve, or stop discomfort in the subject. In some embodiments, the compounds prevent, relieve, or stop itching or pain in the subject.

### Compositions Comprising Activator(s) of MrgprB4⁺ Neurons

In some embodiments, a method of treatment of anxiety comprises administration of an effective amount of a composition comprising one or more activators of MrgprB4⁺ neurons. The activator of MrgprB4⁺ neurons, in some embodiments, is an MrgprB4 agonist, which has been identified as having the ability to stimulate positive valence behavior. In some embodiments, a therapeutic amount of the activator of MrgprB4⁺ neurons is administered to a patient identified as suffering from anxiety. In some embodiments, the composition comprising one or more activators of MrgprB4⁺ neurons is administered peripherally. In some embodiments, the composition is administered topically. In some embodiments, the composition is administered directly to small diameter sensory neurons in DRG and trigeminal ganglia. In some embodiment, the composition is applied on the skin surface of the subject.

In some embodiments, the composition comprising one or more activators of MrgprB4⁺ neurons is used for causing a positive and/or desired effect on a subject. For example, the composition can positively impact the mood of the subject; prevent, reduce or stop anxiety in the subject; prevent, relieve or stop stress in the subject; cause pleasurable effect on the subject; induce or enhance pleasant feeling of the subject; and/or prevent, relieve or stop one or more undesired sensations (e.g., itching and pain) in the subject. In some embodiments, administration of the composition to a subject can lead to positive-valence behavior in the subject. In some embodiments, administration of the composition to a subject makes the subject relax and/or feel comfortable. In some embodiments, administration of the composition to a subject prevents, relieves, or stops discomfort in the subject. In some embodiments, administration of the composition to a subject prevents, relieves, or stops itching or pain in the subject. In some embodiments, administration of the composition to a subject induces or enhances positive-valence behavior in the subject.

In some embodiments, the composition comprises at least one activator of MrgprB4⁺ neurons. In some embodiments, the activator of MrgprB4⁺ neurons is a small molecule. For example, the activator of MrgprB4⁺ neurons can be a small molecule compound capable of activating MrgprB4. In some embodiments, the composition comprises one or more activators of MrgprB4⁺ neurons. In some embodiments, the activator of MrgprB4⁺ neurons is a positive allosteric modulator of a second MrgprB4 agonist. In some embodiments the positive allosteric modulator is administered in combination with the second MrgprB4 agonist.

In still other embodiments, the composition comprises an activator of MrgprB4⁺ neurons that is a nucleic acid. For example, the MrgprB4 neuron activator can be a nucleic acid that binds to the regulatory sequence of MrgprB4 gene and increases the transcription of MrgprB4 gene. As another example, the activator of MrgprB4⁺ neurons can be a nucleic acid that can decrease the degradation of MrgprB4 mRNA.

Therapeutic compositions can comprise any activators of MrgprB4 identified by the methods described herein, and combinations thereof. In some embodiments, the activator of MrgprB4 is included in an amount suitable for reducing, preventing, and inhibiting anxiety. In some embodiments, the activator of MrgprB4⁺ neurons is combined with other ingredients that are suitable for reducing, preventing and inhibiting anxiety. The therapeutic composition can positively impact the mood of the subject; prevent, reduce or stop anxiety in the subject; prevent, relieve or stop stress in the subject; cause pleasurable effect on the subject; induce or enhance pleasant feeling of the subject; and/or prevent, relieve or stop one or more undesired sensations (e.g., itching and pain) in the subject. In some embodiments, administration of the therapeutic composition to a subject can lead to positive-valence behavior in the subject. In some embodiments, administration of the therapeutic composition to a subject makes the subject relax and/or feel comfortable. In some embodiments, administration of the therapeutic composition to a subject prevents, relieves, or stops discomfort in the subject. In some embodiments, administration of the therapeutic composition to a subject prevents, relieves, or stops itching or pain in the subject. In some embodiments, administration of the therapeutic composition to a subject induces or enhances positive-valence behavior in the subject.

In pharmaceutical dosage forms, the activator of MrgprB4⁺ neurons can be used alone or in appropriate association, as well as in combination with other pharmaceutically active or inactive compounds. The activator of MrgprB4⁺ neurons can be formulated into pharmaceutical compositions containing a single activator of MrgprB4⁺ neurons or a combination of two or more activators of MrgprB4⁺ neurons. For example, a pharmaceutical composition can contain two or more different activators of MrgprB4⁺ neurons. In some embodiments, the pharmaceutical composition contains two or more different activators of MrgprB4⁺ neurons having the same mode of action. For example, a pharmaceutical composition can contain two activators of MrgprB4⁺ neurons where both activators of MrgprB4⁺ neurons are MrgprB4 ligands and activate the MrgprB4 directly. As another example, a pharmaceutical composition can contain two activators of MrgprB4⁺ neurons where one of the activators of MrgprB4⁺ neurons is a ligand of MrgprB4 to active MrgprB4 directly and the other activator of MrgprB4⁺ neurons is a positive allosteric modulator of MrgprB4 that increases the activity of MrgprB4 indirectly via activation of an allosteric site on MrgprB4. In some embodiments, the pharmaceutical composition can contain two or more activators of MrgprB4⁺ neurons having different methods of action.

The activator of MrgprB4⁺ neurons can be formulated into pharmaceutical compositions by combination with appropriate, pharmaceutically acceptable carriers or diluents (Remington, The Science and Practice of Pharmacy, 19th Edition, Alfonso, R., ed., Mack Publishing Co., Easton, Pa. (1995), and can be formulated into preparations in solid, semi-solid, liquid or gaseous forms, such as tablets, capsules, powders, granules, ointments, solutions, suppositories, injections, inhalants and aerosols depending on the particular circumstances.

The pharmaceutically acceptable excipients, such as vehicles, adjuvants, carriers or diluents, are readily available. Moreover, pharmaceutically acceptable auxiliary substances, such as pH adjusting and buffering agents, antioxidants, low molecular weight (less than about 10 residues) polypeptides, tonicity adjusting agents, stabilizers, wetting agents and the like, are readily available. “Carriers” when used herein refers to pharmaceutically acceptable carriers, excipients or stabilizers which are nontoxic to the cell or mammal being exposed to the carrier at the dosages and concentrations used.

An activator of MrgprB4⁺ neurons to be used for in vivo administration is preferably sterile. The sterility can be accomplished by any method known in the art, such as by filtration using sterile filtration membranes, prior to or following lyophilization and reconstitution. The activator of MrgprB4⁺ neurons can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

In addition to the formulations described above, the activator of MrgprB4⁺ neurons can also be formulated as a depot preparation. Such long-acting formulations can be administered by implantation (for example, subcutaneously or intramuscularly) or by intramuscular injection. Thus, for example, the activator of MrgprB4⁺ neurons can be formulated with suitable polymeric or hydrophobic materials (for example as an emulsion in an acceptable oil) or ion exchange resins, or as sparingly soluble derivatives, for example, as a sparingly soluble salt.

For oral preparations, the activator of MrgprB4⁺ neurons can be combined with appropriate additives to make tablets, powders, granules or capsules. For example, the activator of MrgprB4⁺ neurons can be combined with conventional additives such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents. Liquid preparations for oral administration can take the form of, for example, solutions, syrups or suspensions, or they can be presented as a dry product for constitution with water or other suitable vehicle before use. Preparations for oral administration can be suitably formulated to give controlled release of the active compound.

The activator of MrgprB4⁺ neurons can also be aerosolized or otherwise prepared for administration by inhalation. For example a fluorocarbon formulation and a metered dose inhaler, or inhaled as a lyophilized and milled powder. For administration by inhalation, the agonists of Mrgprs can be utilized in aerosol formulation to be administered via inhalation. The agonists of Mrgprs can also be formulated into pressurized acceptable propellants such as dichlorodifluoromethane, propane, nitrogen and the like.

In addition, the activator of MrgprB4⁺ neurons can be prepared in a topical composition. In some embodiments, the topical composition is a cosmetic topical composition. Dosage forms for topical administration include, but not limited to, creams, lotion, foam, transdermal patch, powder, gels, ointments and topical sprays. The activator of MrgprB4⁺ neurons can be admixed with a physiologically acceptable carrier and any preservatives, buffers, or propellants as may be required. Ophthalmic formulations, eye ointments, powders, and solutions, as well as dental formulations containing appropriate flavors and sweeteners, are also contemplated as being within the scope of the present disclosure. The topical composition can be packaged in a spray bottle or other suitable delivery device and can be applied to the surface of the skin utilizing a cotton swab, gauze pad, or other suitable applicator.

If an activator of MrgprB4⁺ neurons is co-administered with another activator of MrgprB4⁺ neurons, or with another agent having similar biological activity, the different active ingredients can be formulated together in an appropriate carrier vehicle to form a pharmaceutical composition. Alternatively, the activator of MrgprB4⁺ neurons can be formulated separately and administered simultaneously or in sequence.

The compositions can, if desired, be presented in a pack or dispenser device that can contain one or more unit dosage forms containing the active ingredient. The pack can for example comprise metal or plastic foil, such as a blister pack. The pack or dispenser device can be accompanied by instructions for administration.

In some embodiments, the activator of MrgprB4⁺ neurons is formulated for cellular use, and need not be formulated for administration to a subject. In some embodiments, the activator of MrgprB4⁺ neurons is formulated for direct application into the brain, e.g., direct injection or pump based delivery systems and methods. In some embodiments, the activator of MrgprB4⁺ neurons is formulated for or applied via intraventricular application.

### Methods of Treating Anxiety

In some embodiments, methods of treating (including preventing, (meaning reducing the risk of or time of onset of) an individual suffering from or at risk of anxiety. The methods generally comprise administering to the individual one or more activators of MrgprB4⁺ neurons. In some embodiments, the method comprises identifying a subject suffering from anxiety. In some embodiments, a composition is administered that comprises one or more activators of MrgprB4⁺ neurons at an effective dose. In some embodiments, the composition is administered peripherally. In other embodiments, the composition is administered topically. In some embodiments, the composition is applied on the skin surface of the subject.

A variety of subjects are treatable. Generally, such subjects are mammals, where the term is used broadly to describe organisms which are within the class mammalia, including the orders carnivore (for example, dogs and cats), rodentia (for example, mice, guinea pigs and rats), and primates (for example, humans, chimpanzees and monkeys). In some embodiments, the subjects are humans.

The activator of MrgprB4⁺ neurons can be administered using any convenient protocol capable of resulting in the desired therapeutic activity. A specific protocol can readily be determined by a skilled practitioner without undue experimentation based on the particular circumstances. Thus, the activator of MrgprB4⁺ neurons can be incorporated into a variety of formulations for therapeutic administration, as discussed herein, depending on the protocol adapted by the supervising clinician. In some embodiments, the activator of MrgprB4⁺ neurons, such as an MrgprB4 agonist, can be dissolved in saline solution and delivered directly or indirectly into the spinal cord.

Each dosage for human and animal subjects preferably contains a predetermined quantity of one or more activators of MrgprB4⁺ neurons calculated in an amount sufficient to produce the desired effect, in association with a pharmaceutically acceptable diluent, carrier or vehicle. Again, the actual dosage forms will depend on the particular compound employed, the effect to be achieved, and the pharmacodynamics associated with each compound in the host.

Administration of activator of MrgprB4⁺ neurons can be achieved in various ways, including intracranial, for example injection directly into the brain tissue or into the spinal cord, into the cerebrospinal fluid, oral, buccal, rectal, parenteral, intraperitoneal, intradermal, transdermal, intracheal, intracerebral, etc., administration. The activator of MrgprB4⁺ neurons can be administered alone or in combination with one or more additional therapeutic agents. Administration “in combination with” one or more further therapeutic agents includes both simultaneous (at the same time) and consecutive administration in any order.

Administration can be chronic or intermittent, as deemed appropriate by the supervising practitioner, particularly in view of any change in the disease state or any undesirable side effects. “Chronic” administration refers to administration of one or more activators of MrgprB4⁺ neurons in a continuous manner while “intermittent” administration refers to treatment that is not done without interruption.

Combinations of activators of MrgprB4⁺ neurons for simultaneous administration are used in some embodiments. For example, two or more different activators of MrgprB4⁺ neurons can be administered in combination.

An effective amount of an activator of MrgprB4⁺ neurons to be employed therapeutically will depend, for example, upon the therapeutic objectives, the route of administration, the nature of the activator of MrgprB4⁺ neurons, and the condition of the patient. Accordingly, it can be useful for the therapist to titer the dosage and modify the route of administration as required to obtain the optimal therapeutic effect. A typical daily dosage can range from about 0.01 μg/kg to up to about 1 mg/kg or more, depending on the factors mentioned above. Preferably, a typical daily dosage ranges from about 1 μg/kg to about 100 μg/kg. Typically, the clinician will administer an activator of MrgprB4⁺ neurons until a dosage is reached that provides the best clinical outcome. The progress of this therapy is easily monitored by conventional assays. In some embodiments, a typical daily dosage of activator of MrgprB4⁺ neurons, is from about 1 μM to about 10 mM. In some embodiments, a typical daily dosage of the activator of MrgprB4⁺ neurons is from about 10 μM to about 1 mM, or from about 50 μM to about 0.8 mM, from about 100 μM to about 0.5 mM, from about 200 μM to about 400 μM, or from about 300 μM to about 350 μM.

Toxicity and therapeutic efficacy of an activator of MrgprB4⁺ neurons can be determined by standard pharmaceutical procedures in cell cultures or experimental animals, for example, by determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. The activators of MrgprB4⁺ neurons exhibiting large therapeutic indices are preferred. While compounds that exhibit toxic side effects can be used, care can be taken to design a delivery system that targets such compounds to the site of affected tissue in order to minimize undesired side effects.

### Kits

The compositions disclosed herein, particularly compositions comprising activators of MrgprB4⁺ neurons for preventing, reducing, or stopping anxiety may be assembled into kits or pharmaceutical or cosmetic systems for use in ameliorating, treating, preventing, or stopping anxiety. Kits or pharmaceutical systems according to this aspect of the present disclosure comprise a carrier means, such as a box, carton, tube or the like, having in close confinement therein one or more container means, such as vials, tubes, ampules, bottles and the like. The kits or pharmaceutical systems disclosed herein may also comprise associated instructions for using the agents disclosed herein to prevent, reduce or stop anxiety. The practice of the methods, compositions, kits or systems disclosed herein employs, unless otherwise indicated, conventional techniques, which are well within the purview of the skilled artisan.

### Methods for Identifying Activating Stimuli for Sensory Neurons

Also disclosed herein are methods for identifying stimuli that can activate sensory neurons, for example MrgprB4⁺ neurons. The methods disclosed herein can be used to link molecular identity to stimulus selectivity, for example for primary sensory neuron subtypes that cannot be easily functionally characterized using conventional approaches. As disclosed herein, some sensory neurons, for example MrgprB4⁺ neurons, cannot be detectably activated by sensory stimulation of the skin ex vivo. For example, in isolated skin-nerve preparations, MrgprB4⁺ neurons are not electrophysiologically activated by mechanical, thermal or chemical stimuli.

In some embodiments, the method for identifying activating stimuli for sensory neurons include: applying a stimulus to a subject, wherein the subject has a population of a subset of sensory neurons; and performing calcium imaging to determine activation of the subset of sensory neurons. Calcium imaging is designed to show Ca²⁺ status of a cell, tissue or medium. The Ca²⁺ status of a cell, tissue or medium, in some embodiments, is an indicator of the activation status of the cell, tissue or medium. The subset of sensory neurons is, in some embodiments, MrgprB4⁺ neurons. In some embodiments, the method further includes identifying the population of the subset of sensory neurons. In some embodiments, the subject has the identified population of the subset of sensory neurons. Conventional methods for identifying a population of a subset of sensory neurons of interest are known in the art. For example, the subset of sensory neurons can be identified using immunofluorescence or immunostaining. In some embodiments, a gene specifically expressed in the subset of sensory neurons is genetically modified to express with a detectable marker (e.g., a visible marker such as fluorescence gene or lacZ, or a drug resistance gene such as neomycin or kanamycin). Calcium imaging techniques use calcium indicators and fluorescent molecules that can respond to the binding of Ca²⁺ ions by changing their fluorescence properties. Non-limiting examples of calcium indicators include chemical indicators, for example smaller molecules that can chelate calcium ions; and genetically encoded calcium indicators (GECI or GCaMP). For GECI, genes encoding for fluorescent proteins derived from green fluorescent protein (GFP) or its variants (e.g., circularly permuted GFP, YFP, CFP), fused with calmodulin (CaM) and the M13 domain of the myosin light chain kinase, which is able to bind CaM Calcium imaging can be used to optically probe intracellular calcium in living animals. Non-limiting examples GCaMP include GCaMP3 and GCaMP5. In some embodiments, the calcium imaging is two-proton calcium imaging. In some embodiments, the sensory neurons are MrgprB4⁺ neurons.

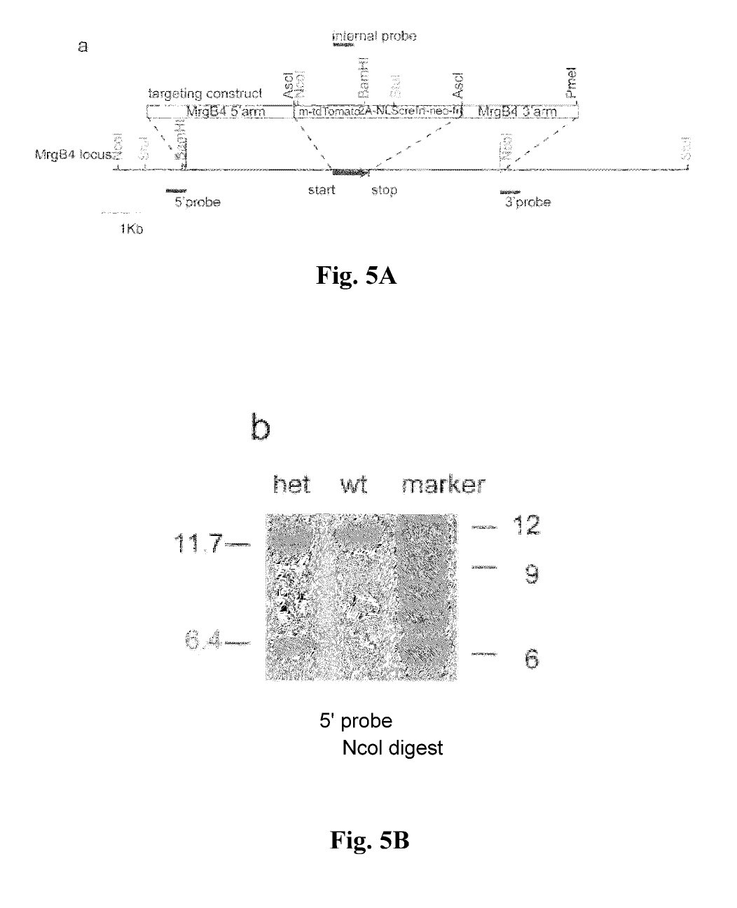

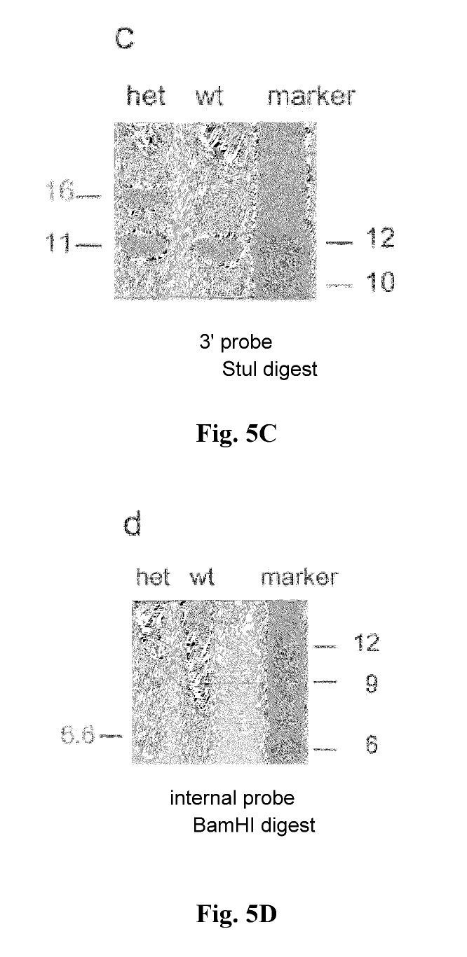

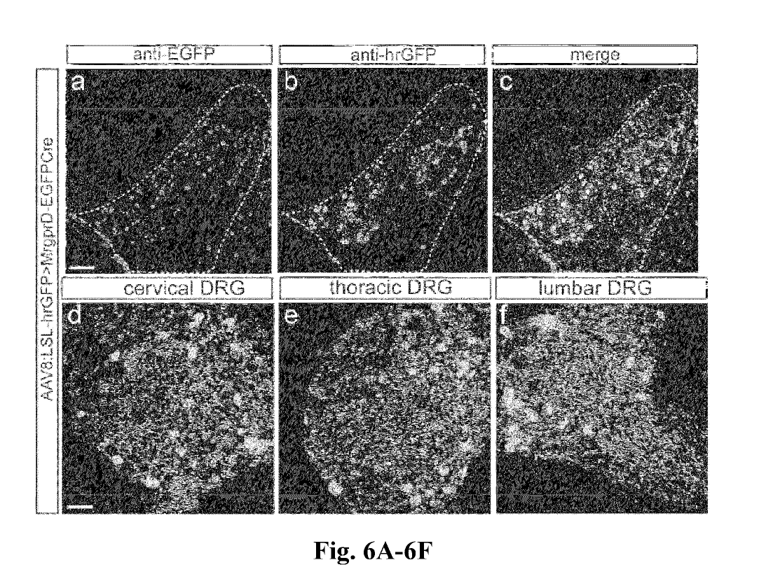

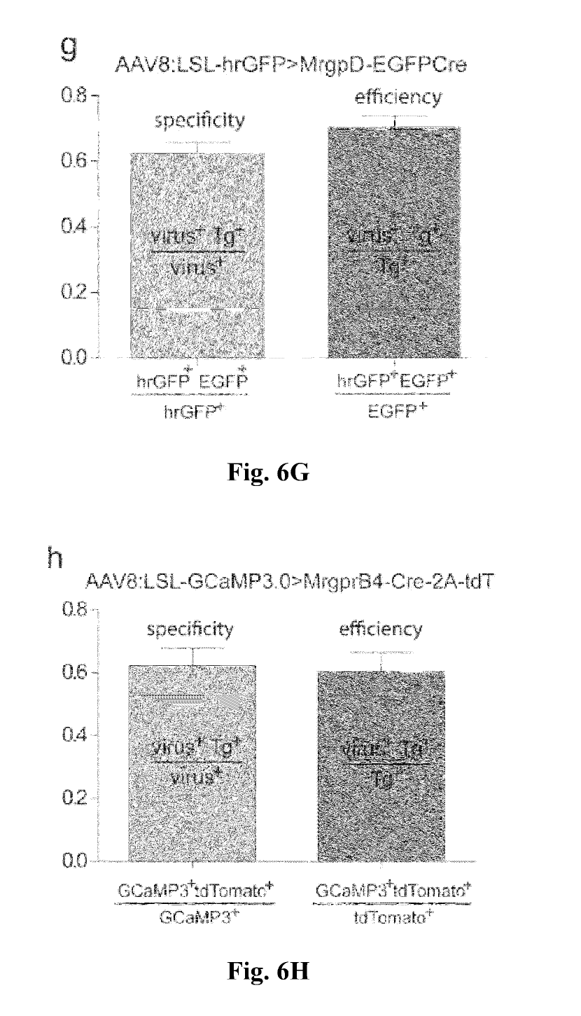

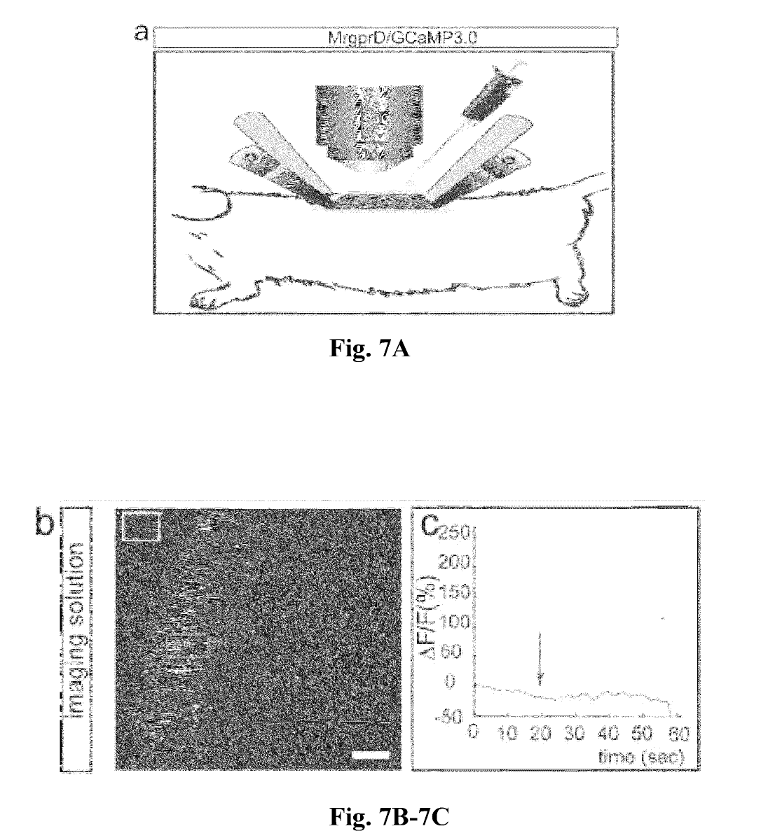

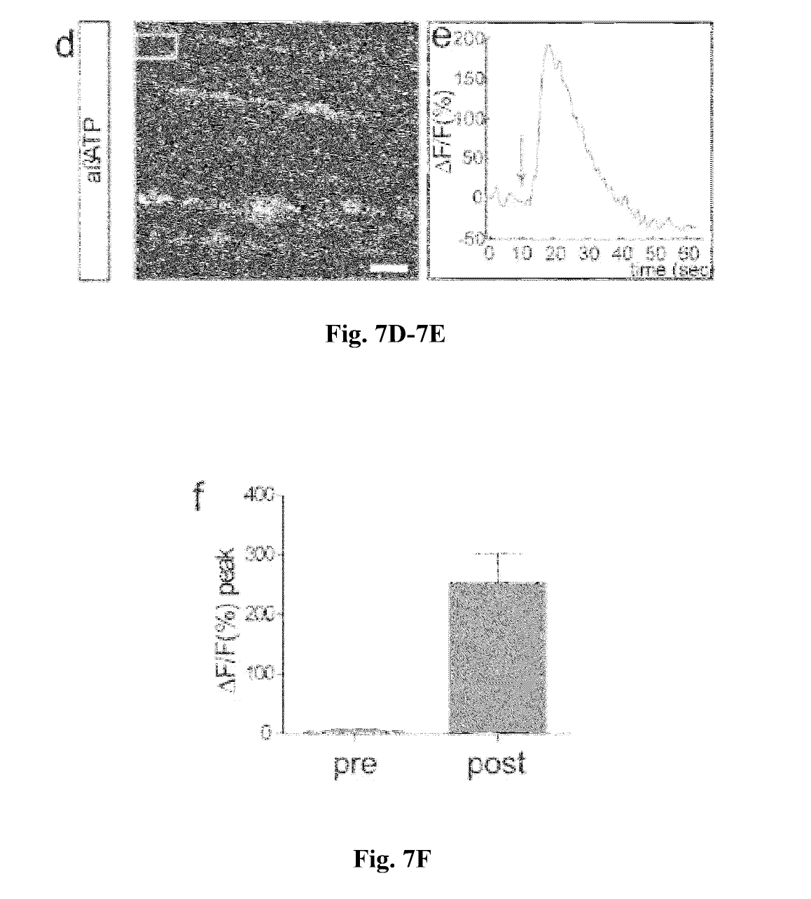

In some embodiments, the sensory neurons (e.g., MrgprB4⁺ neurons) are genetically modified. For example, the sensory neurons can be genetically modified to express one or more fluorescent proteins as calcium indicator(s). In some embodiments, the sensory neurons are genetically modified to express a GCaMP. The method by which the sensory neurons are genetically modified is not particularly limited. For example, the genetic modification can be carried out by introducing a nucleic acid sequence encoding a GCaMP to the subject having a population of the subset of sensory neurons, for example by administering a viral vector comprising a nucleic acid sequence encoding a GCaMP to the subject. The viral vector can incorporate sequences from the genome of any known organism. Various promoters can be operably linked with the nucleic acid encoding a GCaMP in the viral vectors disclosed herein. In some embodiments, the nucleic acid sequence encoding a GCaMP in the viral vector is flanked by loxP sites. In some embodiments, the viral vector comprises at least a portion of an Mrgpr gene (e.g., MrgprB4 gene). In some embodiments, the viral vector comprises two portions of an Mrgpr gene (e.g., MrgprB4 gene), where the two portions of the Mrgpr gene are separated by a nucleic acid sequence comprising a sequence encoding one or more marker genes. In some embodiments, the viral vector comprises two portions of MrgprB4 open reading frame, wherein the two portions of MrgprB4 open reading frame are separated by a nucleic acid sequence encoding one or more marker genes. Non-limiting examples of marker genes include drug-resistant markers (e.g., neomycin resistance cassette, kanamycin resistance cassette), fluorescence gene, and lacZ gene. In some embodiments, the viral vector is used to replace a portion of or a whole Mrgpr receptor gene (e.g., MrgprB4 gene) from the genome of one or more cells. In some embodiments, the viral vector is used to replace a portion of or a whole open reading frame of an Mrgpr receptor gene (e.g., MrgprB4 gene) from the genome of one or more cells. A non-limiting example of the viral vector is illustrated schematically in FIG. 5. Various posttranscriptional regulatory elements can be used in the viral vectors, for example to increase expression level of the protein of interest in a host cell. Non-limiting examples of viral posttranscriptional regulatory element include woodchuck hepatitis virus posttranscriptional regulatory element (WPRE), hepatitis B virus posttranscriptional regulatory element (HBVPRE), RNA transport element (RTE), and any variants thereof.

The administration of viral vector can be carried out by injection, for example, intramuscular, intra-peritoneal or intravenous injection. To increase the transfection efficiency, it is preferred that the viral vector is administrated to the subject when the subject is at the stage of neonatal pups. For example, the viral vector can be administered to the subject when the subject is about day 1, day 2, day 3, day 4, day 5, day 6, or day 7 old. In some embodiments, the viral vector is administered to a P1 or P2 pups of the subject (e.g., a P1 or P2 mouse pups). Without being bound to a particular theory, it is believed that administration of the viral vector encoding a GCaMP to the subject at the stage of neonatal pups can avoid a highly localized distribution of infected cells whose peripheral receptive fields may be equally restricted. In some embodiments, the viral vector is derived from adeno-associated virus (AAV), for example AAV serotype 8 (AAV8). In some embodiment, the calcium imaging (for example the two-proton calcium imaging) is carried out on the skin of the animal.

In some embodiments, Cre-LoxP recombination system is used for generating a subject having genetically modified sensory neurons (e.g., MrgprB4⁺ neurons). For example, the viral vector comprising the nucleic acid sequence encoding GCaMP can administered to a subject that has Cre recombinase predominantly or only expressed in one or more specific cell types, or one or more specific tissues. For example, the subject can have Cre recombinase expressed in brain, ganglia, or a subset of sensory neurons of interest in a greater extent as compared to other tissues, cells, or neurons. In some embodiments, the subject can have Cre recombinase predominantly or only expressed in brain, ganglia, or a subset of sensory neurons of interest. In some embodiments, the subject has Cre recombinase predominantly or only expressed a subset of sensory neurons. For example, the subject can have Cre recombinase expressed in a subset of sensory neurons, but not in other types of sensory neurons. In some embodiments, the subject has Cre recombinase predominantly or only expressed in MrgprB4⁺ neurons. The Cre recombinase can be consitutently or inducibly expressed in the subject. For example, the Cre recombinase can be expressed under the control of an inducible or other conditionally active promoter. The inducible promoter can be, for example, a temperature-inducible, a physically-inducible promoter, or a chemical-inducible promoter. For example, the expression of the Cre recombinase can be induced by the presence of one or more chemical compound selected from isopropyl β-D-1-thiogalactopyranoside (IPTG), rhamnose, arabinose, xylose, fructose, melbiose, and tetracycline. In some embodiments, the expression of the gene encoding the Cre recombinase is induced by a change in temperature. In some embodiments, the expression of the gene encoding the Cre recombinase is induced by the presence or absence of one or more physical factors, such as water or salt stress, illumination, light or darkness, radiation, low or high temperatures, oxygen, and nitrogen. Without being bound to any particular theory, it is believed that cell type-specific or tissue-specific expression of Cre recombinase can improve the efficiency and specificity of gene targeting, for example the replacement of a Mrgpr gene, using Cre-LoxP recombination system. For example, if a viral vector encoding a GCaMP flanked by loxP sites is administered to a subject expressing Cre recombinase predominantly or only in ganglia, the Cre-LoxP facilitated recombination event will occur specifically in ganglia. A non-limiting example of the use of Cre-LoxP recombination system to generate MrgprB4 knock-in mice is described in the Example section below, for example the section entitled Generation of Mrgprb4 knock-in mice.

The type of stimuli that can be tested in the methods disclose herein is not particularly limited. For example, the stimulus can be a mechanical stimulus (e.g., touching, pinching, massaging, pressure, pin-pricking, joint movement, brushing, grooming, or stroking), a thermal stimulus (e.g., adjustment of temperature, radiation, cold thermal stimulus, or heat thermal stimulus (e.g., intense heat)), a chemical stimulus (e.g., odorants, acids, alkaline, small molecules, proteins, or nucleic acids), or a combination thereof. In some embodiment, the stimulus is pinching, stroking, brushing, grooming, or massaging.

As described herein, the method for identifying activating stimuli for sensory neurons can, in some embodiments, include preparing a viral vector comprises two portions of a Mrgpr gene open reading frame (e.g., MrgprB4 open reading frame) and encodes a genetically encoded calcium indicator, wherein the two portions of the Mrgpr open reading frame are separated by a nucleic acid sequence encoding one or more marker genes; administering the viral vector to a Cre-expressing subject wherein a Cre recombinase gene under the control of an inducible promoter and is expressed predominantly or only in a subset of sensory neurons of interest (e.g., MrgprB4⁺ neurons) to prepare a Mrgpr gene knock-in subject; applying a stimulus to the Mrgpr gene knock-in subject; and performing two-proton calcium imaging to determine activation of the subset of sensory neurons.

The way that the stimulus can be applied to the subject can vary. For example, the stimulus can be applied centrally or peripherally to the subject. In some embodiment, the stimulus is applied to the subject peripherally. In some embodiment, the stimulus is applied to the skin (e.g., skin surface) of the subject.

### EXAMPLES

Some aspects of the embodiments discussed above are disclosed in further detail in the following examples, which are not in any way intended to limit the scope of the present disclosure.

### Experimental Materials and Methods

The following experimental methods were used for Examples 1-6 described below.

### Methods Summary

Mice expressing reporters and/or Cre recombinase targeted to the MrgprB4 locus were generated by homologous recombination in embryonic stem cells, according to standard procedures. Heterozygous neonates from MrgprD-Cre and MrgprB4-Cre mice were injected intraperitoneally with Cre-dependent AAV8 viruses expressing GCaMP3.0 or mGCaMP3.0 or hM3(Gq-coupled) DREADD, and imaged as adults (≧8 weeks old).

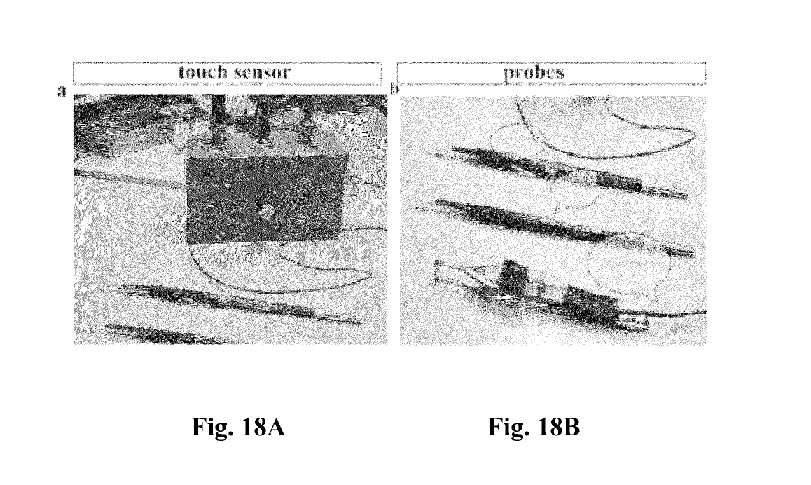

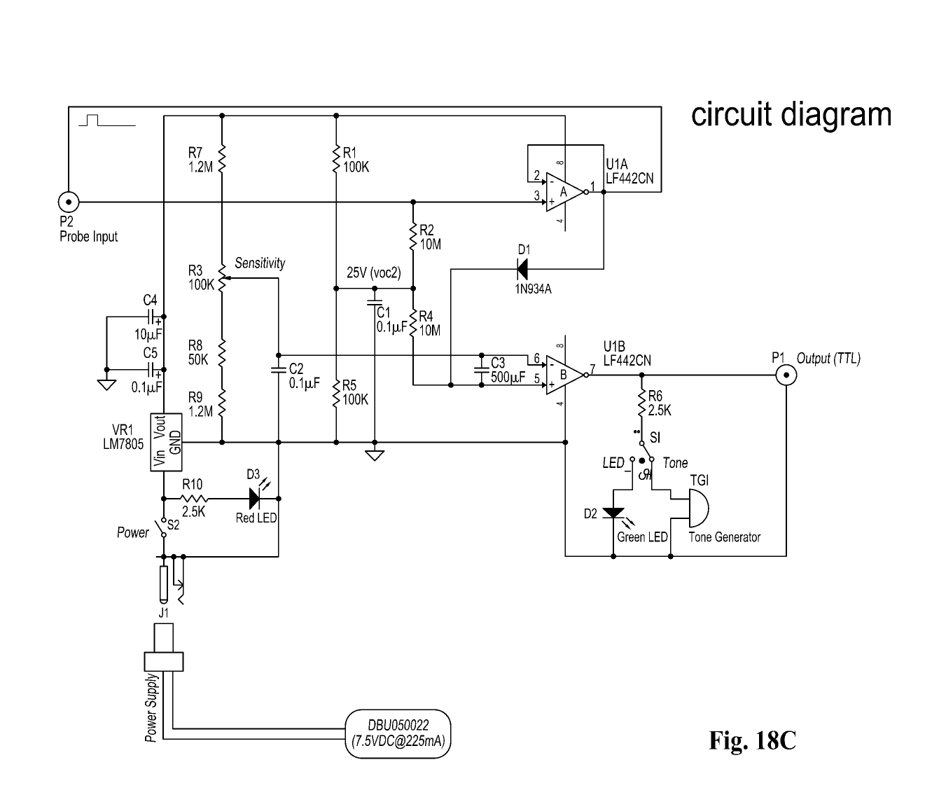

Electrophysiology experiments on ex vivo skin-nerve preparations from adult heterozygous Mrgprb4-EGFPf reporter mice were performed as described in Woodbury, et al. J. Comp. Neurol. 436, 304-323 (2001). For calcium imaging, after a dorsal laminectomy the spinal column was stabilized and filled with imaging solution (see below for details). Imaging was performed using a two-photon laser-scanning microscope (Ultima, Prairie Inc.) using an Olympus 40×0.8 N.A. water immersion objective, at 128×128 pixel resolution with an acquisition rate of 8-12 frames per second. Mechanical stimuli were delivered using a custom-modified No. 5 sable paint brush or serrated forceps, in a manner electronically time-stamped to image acquisition, as may be seen in FIGS. 18A-18C and further discussed below. Stimuli were delivered to each mouse in a series of trials, separated by a few minutes; each trial consisted of one or more stimuli delivered typically at intervals of several seconds. Chemical stimuli were delivered to the spinal cord using a Pipetman pipette and to the periphery using a syringe pump. Calcium responses were analysed using custom software written in Matlab (see below for more details). For calculating ΔF/F [(Fav−F0)/F0], F₀ is the average of the first ten frames of the recording period.

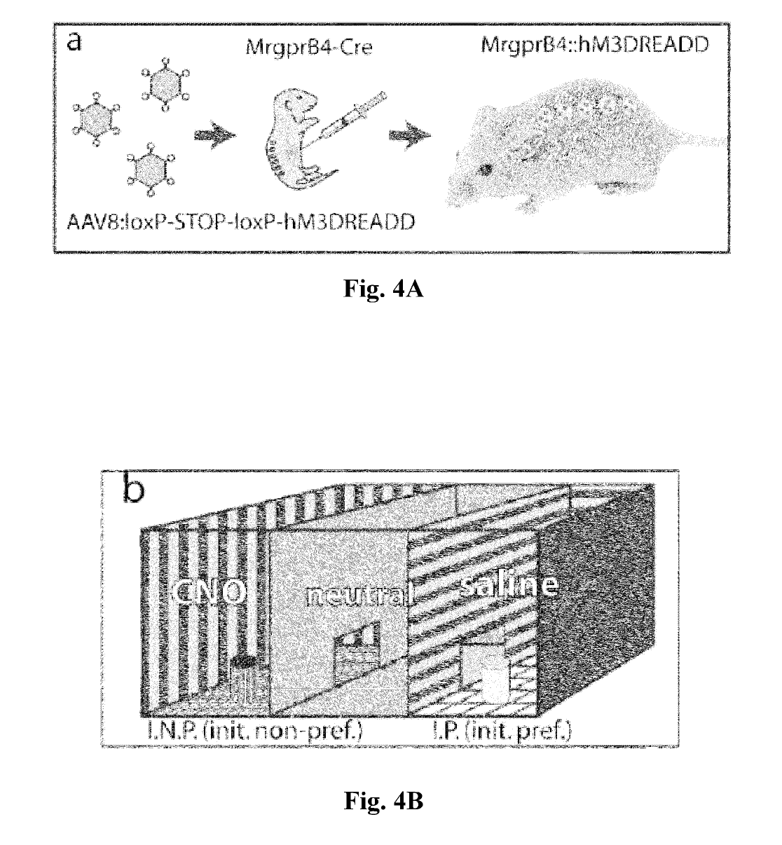

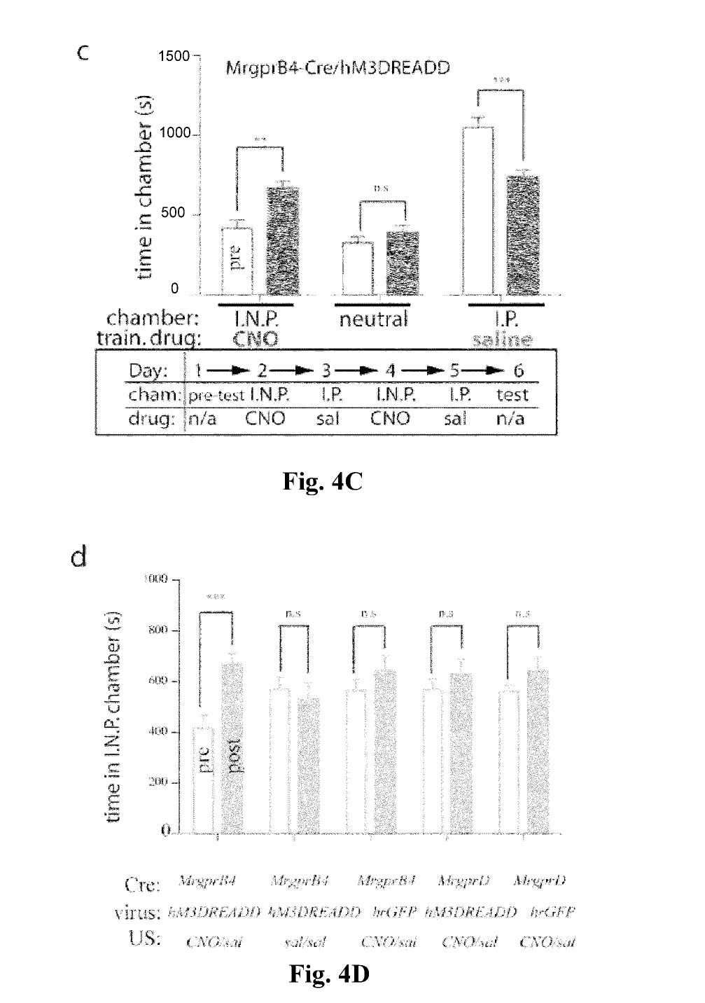

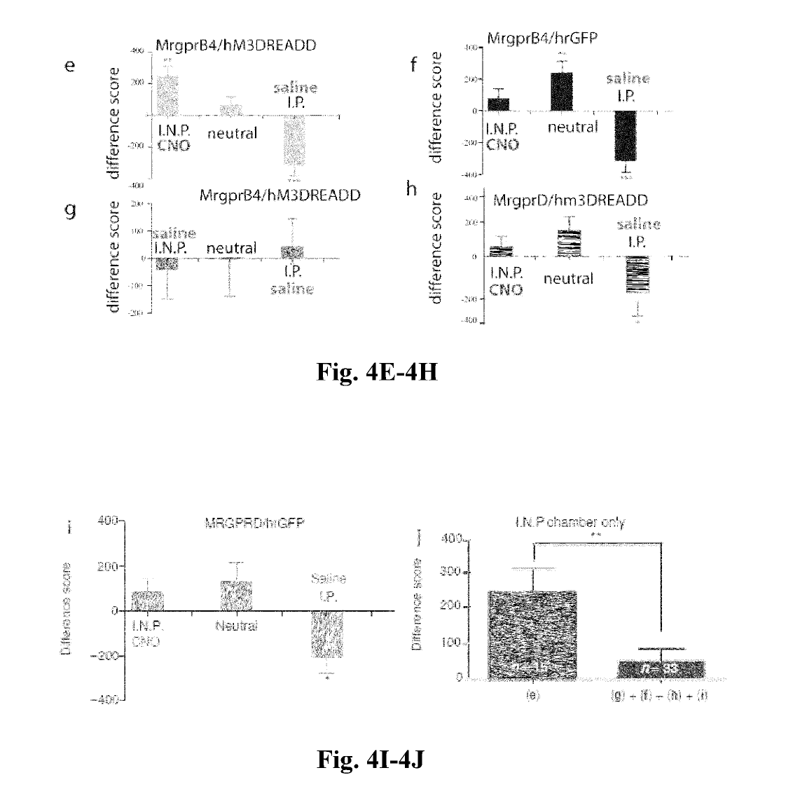

For behavioral experiments, juvenile (1-month-old) mice neonatally injected with Cre-dependent AAV8 viruses expressing hM3DREADD were subjected to a conditioned place preference assay (CPP13) using a biased design, by an investigator blind to genotype. All mice were tested for their initial chamber preference before conditioning, as depicted in FIG. 4C, lower panel).

All data were analyzed for statistical significance using repeated measure ANOVAs (unless stated otherwise). After detection of a significant interaction and/or main effect, Bonferroni-corrected post-hoc comparisons of means were performed. Further details of the statistical analysis are discussed below.

### Animals

Animals were group-housed, unless otherwise mentioned, at 23° C. with ad libitum access to food and water in a 13-h/11-h light/dark cycle, with the day starting at 07:00. All animal procedures were performed under protocols approved by the Caltech Institutional Animal Care and Use Committee (IACUC).

### Generation of Mrgprb4 Knock-in Mice.

Mrgprb4-mtdTomato-2A-NLScre-frt-PGK-neo-frt and Mrgprb4-EGFPf-2A-FLP-ACN mice were generated via standard gene-targeting methods in embryonic stem cells, using the 129/SvJ targeting arms of MrgprB4 as described in Liu, Q. et al. Nature Neurosci. 10:946-948 (2007). The lengths of 5′ and 3′ arms were 4.3- and 3.0 kb, respectively. In one construct, the entire open reading frame of MrgprB4 (encoded by a single exon) was replaced with an mtdTomato-2A-NLSCre targeting cassette. This cassette was generated as a single open reading frame using overlapping PCR that connected the membrane-tagged tdTomato (containing the 8 amino acids of the MARCKS sequence (MGCCFSKT (SEQ ID NO: 1)) fused to the amino terminus of the full-length tdTomato, including its N-terminal methionine) to a nuclear localization signal (NLS)-tagged Cre-recombinase via an intervening F2A sequence. This cassette was ligated as a SacII/SalI fragment to the frt-PGK-neo-frt cassette. It was then ligated in-frame to an AscI site at the endogenous ATG start codon of the Mrgprb4 coding sequence. To generate MrgprB4-EGFPf-2A-FLP-ACN mice, the open reading frame of MrgprB4 was replaced by the EGFPf-2A-FLP cassette, where EGFPf (farnesylated EGFP; Clontech) was fused via the 2FA sequence to FLPo (codon optimized FLP recombinase). This cassette was ligated to the self-excising loxP-flanked pol-II promoter-neomycin resistance cassette (ACN).

Homologous recombination was performed in mouse CJ7 embryonic stem (ES) cells following standard procedures. Correctly targeted ES clones were identified by PCR genotyping of genomic DNA isolated from G418-resistant clones using primer sets flanking the 5′ and 3′ arms of the targeting construct and were further confirmed by Southern blot hybridization using probes that flanked the 5′ and 3′ arms of the targeting construct, as well as an internal probe to exclude illegitimate recombination events. Chimeric MrgprB4-mtdTomato-2A-NLScre-frt-PGK-neo-frt and MrgprB4-EGFPf-2A-FLP-ACN mice were produced by blastocyst injection of positive ES cells, and heterozygous progeny were generated by mating the chimaeric mice to C57BL/6N mice. Back-crossing to C57BL/6N mice was done for five or more generations.

### Neonatal Mouse Viral Injections

P1-P2 pups were removed from their cage and briefly submerged in an ice water bath inside a latex glove with their head up, until they appeared to be anaesthetized (3-5 min). The adequacy of anesthesia was determined by toe pinch. Pups were then held gently by the head, with padding, the skin of the lower abdomen cleaned with an alcohol swab, and the animals were then immobilized in a plastic gel pocket with their ventral side up. A syringe (insulin syringe, 0.3 cm³, 8 mm length, 31G needle) was used to inject AAV8 virus (20-25 ml containing 10¹⁰ AAV8 particles), titred by dot-blot hybridization or by genome copy number (using quantitative real time PCR, qPCR) intraperitoneally (i.p.), avoiding any visible milk spot. The pups were then covered with nesting material and placed on a water circulating heating pad until they began moving. After this recovery period they were returned to their dam and observed for the appearance of a milk spot, indicating that they were healthy and suckling.

### Virus Production

AAV8 virus particles were produced using crude iodixanol purification, as described in Zolotukhin, et al. Gene Ther. 6:973-985 (1999), and concentrated using a Millipore Ultra-15 unit (no. UFC910024).

### Immunofluorescence

Adult mice (8-16 weeks old) were anaesthetized with ketamine/xylazine and perfused with 20 ml 0.1 M phosphate buffer solution (PBS; pH 7.4; 4° C.) followed by 25 ml 4% paraformaldehyde (PFA) in PBS (4° C.). Dorsal root ganglia (DRG) were dissected from the perfused mice, postfixed in 4% PFA at 4° C. for 5 min, cryoprotected in 20% sucrose in PBS at 4° C. for 24 h, and frozen in OCT at −80° C. Tissues were sectioned at 20 μm with a cryostat. The sections collected on slides were dried at 37° C. for 15 min. The slides were washed with PBS containing 0.2% Triton X-100 (PBT) and blocked with 10% goat/donkey serum in PBT for 30 min. All sections were incubated overnight with primary antibodies diluted in blocking solution at 4° C. The primary antibodies used were: rabbit anti-GFP (A-11122; Molecular Probes; 1:1,000), rabbit anti-hrGFP (240142; Stratagene; 1:200) and chicken anti-GFP (GFP1020; Ayes Labs; 1:1,000). After incubation with primary antibody, sections were washed with PBT and incubated with secondary antibodies at room temperature for 2 hr. Secondary antibodies were diluted 1:250 in blocking solution and were conjugated to Alexa 488 or Alexa 568 (Molecular Probes). Sections were counterstained with TO-PRO-3 (Molecular Probes), washed with PBT and mounted with Vectashield. Images were obtained using an Olympus Confocal Microscope system.

### Electrophysiological Recording in Ex Vivo Skin-Nerve Preparations.

The ex vivo somatosensory system preparation has been described in detail in Woodbury, et al. J. Comp. Neurol. 436:304-323 (2001). Briefly, adult Mrgprb4-EGFP-2A-FLP mice were anaesthetized with a mixture of ketamine (90 mg/kg) and xylazine (10 mg/kg), the skin of the dorsal hindpaw and limb was shaved and then the mice were transcardially perfused with chilled and oxygenated artificial cerebral spinal fluid. Surgical dissection was performed to isolate intact the hemisected spinal cord, L2-L3 dorsal roots and DRGs, saphenous nerves and innervated skin from the left or right hindlimbs. The skin was pinned hairy-side up on an elevated platform, keeping the dermal side perfused and the epidermis dry. Bath temperature was maintained at 31° C. EGFP cells were targeted using fluorescent microscopy and DIC optics. Recording electrodes contained 5% neurobiotin (NB) in 1 M potassium acetate. A small amount of <1% lucifer yellow was added to the solution for better visualization of the microelectrode tip under fluorescent illumination. After impalement of a targeted neuron projecting through the saphenous nerve, its receptive field was first searched by stroking the skin using a fine camel-hair brush. Next the skin was searched using a small glass rod. Thermal stimuli were next applied by flooding the skin surface with first cold (0° C.) and then hot (52° C.) buffered saline. Finally, in some of the experiments the skin was then treated with a cocktail of inflammatory compounds (10 μM histamine, 10 μM bradykinin, 10 μM serotonin and 10 μM prostaglandin E2, in 50% DMSO and 50% buffered Krebs solution at pH 6) for 3-5 min to determine if the cells were either chemosensory or whether they could be sensitized to respond to the other stimulus modalities.

### Summary of Electrophysiology Results

25 EGFP+ positive cells were recorded from 10 saphenous nerve preparations made from Mrgprb4-EGFP-2A-FLP mice. None of these 25 cells could be activated with mechanical stimulation of the skin. Of these, 21 were also thoroughly tested for thermal sensitivity and were found to be unresponsive. Finally, four cells were tested with mechanical thermal and chemical stimuli (inflammatory soup) and all four remained unresponsive.

### Calcium Imaging

Mice 2 months or older were sedated by i.p. injection of a mix of ketamine (100 mg/kg), xylazine (15 mg/kg), acepromazine (2.5 mg/kg in 0.9% NaCl). During surgery, body temperature was maintained at 37° C. with a heating blanket.

A dorsal laminectomy was performed mostly at spinal level L2-L4 (but occasionally at L1-L3) as described in Johannssen, et al. J. Physiol. (Lond.) 588:3397-3402 (2010), but without removing the dura. The spinal column was stabilized using Narishige STS-A spinal clamps. In addition a head-holding adaptor from Kopf (923-B Mouse Gas Anaesthesia Head Holder) was used that has installed an anaesthesia/gas mask for positioning the mouse head. In this apparatus the gas is applied through a standard hose barb positioned above the nose on the mask. The inlet fills a large gas chamber around the snout, a second hose barb below the mask is provided for vacuuming off excess, expelled gasses. The animals were maintained under continuous anaesthesia for the duration of the imaging experiments with 1-2% isofluorane or with hourly injections of the above ketamine mix. A well was built around the exposed spinal cord using Gelseal (Amersham Biosciences Corp) and Kwik Sil Adhesive (WPI). Warm imaging solution (in mM: 130 NaCl, 3 KCl, 2.5 CaCl₂, 0.6 6H₂O.MgCl₂, 10 HEPES without Na, 1.2 NaHCO₃, 10 glucose, pH7.45 with NaOH) (37° C.) was repeatedly applied to prevent drying and maintain tissue integrity, and to allow the use of immersion objectives. During imaging the body temperature of the animals was maintained at 37° C. with a heating blanket and an air-therm heater (WPI) placed inside the microscope area.

Imaging experiments were performed under a two-photon laser-scanning microscope (Ultima, Prairie Instruments Inc.). Live images were acquired at 8-12 frames per second, at depths below the pia ranging from 100 to 250 μm, using an Olympus 40×0.8 N.A. water immersion objective, at 128×128 pixel resolution with a laser tuned to 940 nm wavelength, and emission filters 525/50 nm and 595/50 nm for green and red fluorescence, respectively. Laser power was adjusted to be 20-25 mW at the focal plane (maximally 35 mW), depending on the imaging depth and level of expression of GCaMP3.0. Focal planes containing fibres activated by stimulation of a given peripheral area were identified by trial and error. tdTomato fluorescence was used to identify MrgprB4⁺ fibres until photobleaching occurred.

### Stimulus Delivery During Imaging Experiments

Mechanical Stimuli.

Brushing stimuli were delivered using a sable paint brush No. 5. Pinching stimuli were delivered using serrated forceps (Adson-Graefe tissue forceps, Fine Science Tools, catalogue no. 11030-12). A touch sensor was designed to allow detection of a finger touch to a conductive band (copper) mounted on the paint brush. The touch sensor is shown in FIG. 18A and the probes are shown in FIG. 18B. The function of the sensor amplifier/brush is to allow the coordinated movement of the brush tip with a light touch of the sensor band to produce a TTL (+5 VDC) compatible voltage pulse that can be time-stamped to the image acquisition. For the pinching stimulation the touch sensor was modified to detect closure of the forceps. This adjustment was accomplished by mounting a plastic screw on the forceps so that the contact closure occurred at a consistent position, as may be seen in FIG. 18B.

The circuitry inside the touch-sensor box was designed as follows and may be seen in the circuit diagram shown in FIG. 18C: the stimulus device (brush or forceps) was attached by a small wire with a male pin at the end. A 2-m cable with a matching female receptacle and a BNC connector conveyed the electrical signal to the touch-sensor amplifier. The probe input on the amplifier was connected to +2.5 VDC through a 10 MΩ resistor. This point was attached to a high impedance follower. The shield (outer part of the coaxial cable) on the probe wire is ‘driven’ by being connected to the output of the follower. This provides a low-impedance shield to keep electrical interference from coupling to the touch probe input line. When a touch is made, the output of the follower amplifier has a noise envelope (primarily 60/120 Hz) picked up by the body of the person touching the probe band (or the metal body of the forceps). The signal from the follower amplifier is rectified and injected into the positive input of a voltage comparator. The minus input of this circuit is connected to the wiper of a potentiometer on the front panel that provides a sensitivity adjustment. This adjustment allows for the wide range of touch sensitivity that is needed. When the voltage on the plus input (signal from the probe amplifier) exceeds the voltage on the minus input (set by the potentiometer) the output of the comparator is increased. The output of this comparator is conveyed to a BNC connector on the panel as a TTL pulse. The voltage level on this BNC remains high (+5V) as long as the ‘touch’ is being made. The signal is internally directed to a three position switch that allows for an LED to be lit or a tone to be generated, enabling visual or auditory confirmation of times when stimulation is performed. The TTL pulse is recorded by the Trigger Sync program (Prairie) which is time-locked with the two-photon image acquisition system (Prairie View, Prairie), thereby identifying imaging frames at which the mechanical stimuli were applied.

It was concurrently recorded where on the animal the mechanical stimulation was applied. First, a dim red grid was projected onto the mouse (so as to have the least interference with the detection of the green fluorescence) using a laser pico projector (MicroVision, SHOWWX) to deliminate a coordinate system for stimulation. Then the movement of the brush and the location in the peripheral areas of the brushes were recorded using a camera (Basler, A601f-2).

Delivery of Chemical Stimuli to the Spinal Cord.

KCl, final concentration (60 mM), α,β-methyl ATP (5 mM) and CNO (1.5 mM) were delivered manually to the imaging bath using a pipetman pipette.

Delivery of Chemical Stimuli to the Periphery.

α,β-methyl ATP (10 μl from 1 mM solution) and capsaicin (10 μl from 1 mM solution (10% DMSO in saline)) was injected in the ventral and dorsal hindpaw of MrgprD and MrgprB4 mice, respectively, using a syringe pump (WPI, Inc., sp200i syringe pump). The timing of the injection was controlled by the two-photon image acquisition system and associated software (Prairie View and Trigger Sync, Prairie Technologies) to link it with image acquisition.

### Analysis of Imaging Data

GCaMP3.0 responses were quantified using custom software written in Matlab (VivoViewer software). Initially, the raw data were filtered by smoothing using a Gaussian filter. The filter is represented by a 3×3 matrix with values proportional to a two-dimensional Gaussian with its peak at the centre, s.d.=0.5, and normalized so that the matrix's entries sum to 1. The filtered value for each pixel=(its original value×the filter value in the centre)+(original values of the adjacent pixels each multiplied by their corresponding filter values). To calculate values for pixels at the edge of the image, the image is treated as though there are pixels beyond the edge with values equal to those of the nearest edge pixel. Next, the images were subjected to background subtraction to remove excess background noise. This was accomplished by drawing an ROI around a region without any visible structures and calculating the average pixel value in that background ROI, for each frame used for analysis. This value was then subtracted from every pixel in the corresponding frame.

The average fluorescence intensity, Fₐᵥ, was measured by calculating the average (background-subtracted) pixel values in a given ROI, for each image frame recorded during a time interval spanning before and during the stimulation period. The Fav was then converted to ΔF/F using the formula ΔF/F=(Fav−F0)/F0, where F₀ is the baseline fluorescence value, measured as the average pixel intensity during the first 2-11 frames of each imaging experiment. The resulting time series of ΔF/F in a given ROI was smoothed using a moving average with a window of three frames. For a window of size of M the following equation is used: for a time series, f, of N frames and a window size of M for the moving average (where M is an odd integer), the nth term of the new time series, F, is giving by

$${F}_{n} = {\sum }_{i = n - m}^{n + m} \frac{{f}_{i}}{2 m + 1}$$

wherein

$$m = \frac{M - 1}{2}$$

if both

$$n > \frac{M - 1}{2}$$

and

$$n \leq N - \frac{M - 1}{2}$$

are true. Otherwise, m=min{n−1,N−n}.

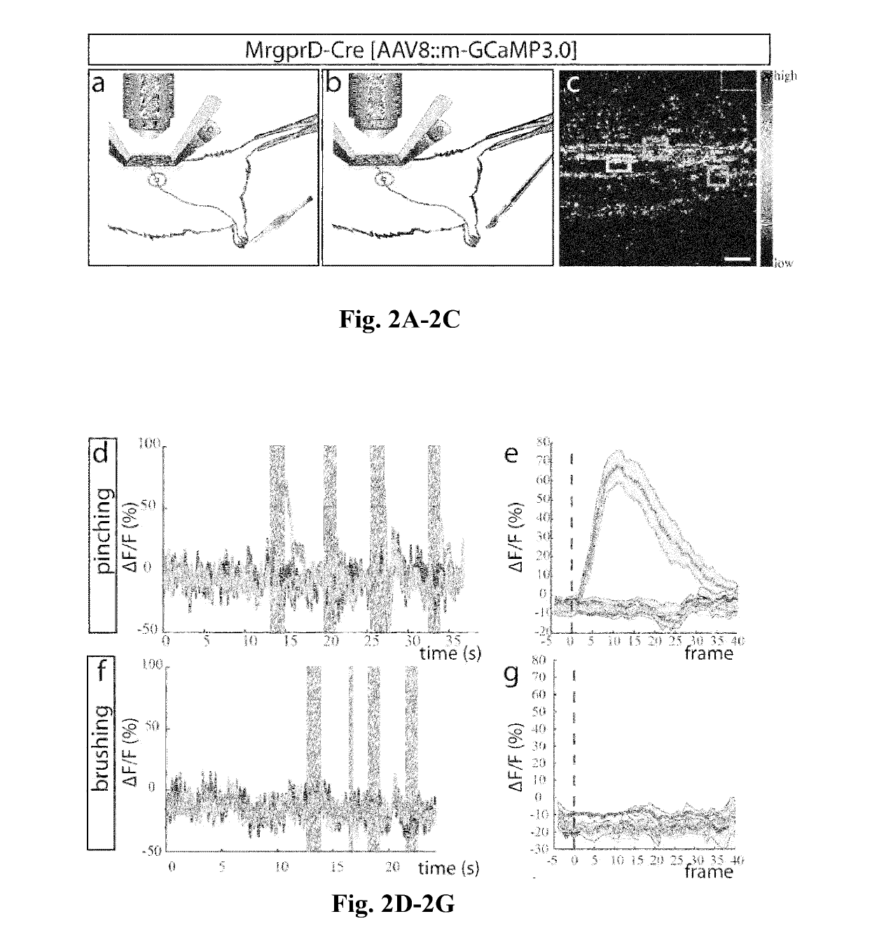

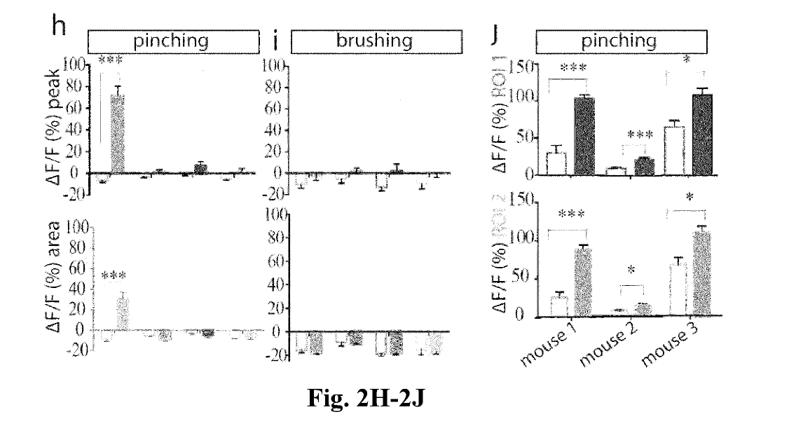

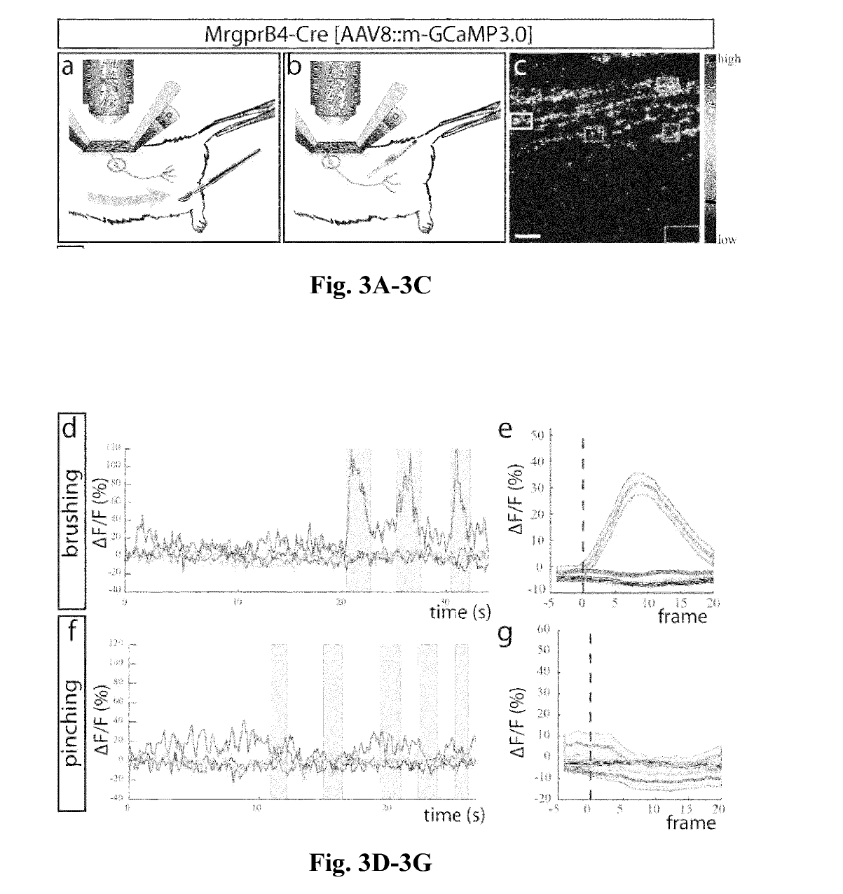

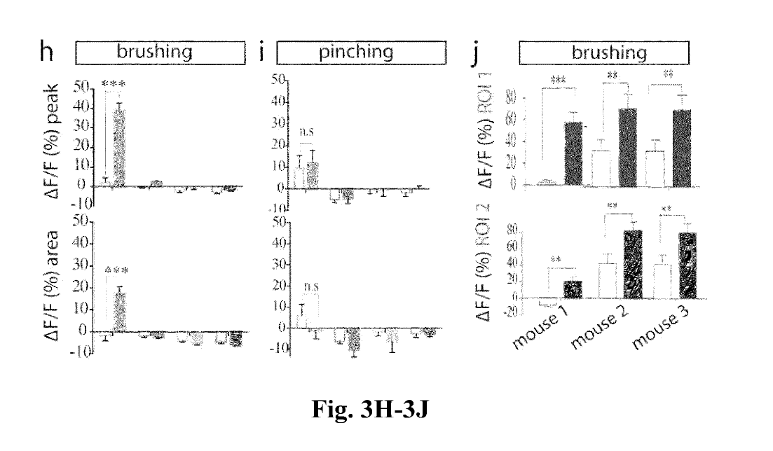

For calculation of the trial average curves (see, for example, FIGS. 2E, 2G, 3E and 3G) for mechanical stimuli, a seven-frame smoothing window was used. Sections of the ΔF/F time series during which a stimulus occurred were collected for multiple trials, aligned to the onset of the stimulus, and averaged to find the mean response curve. Because repeated mechanical stimuli were delivered during each experimental trial, to be consistent each ΔF/F trace was calculated for a period of five frames just before each stimulus onset and for the subsequent 20 or 40 frames (that is, the first frame of these 20-40 frame series coincided with the initiation of the stimulus). From these values the mean peak ΔF/F (MPI ΔF/Fpeak) and area under the curve were calculated for all the applied stimuli across trials. The average ΔF/F values for specific ROIs in the same field of view were tested for statistical significance by repeated measures ANOVA, followed by Bonferroni-corrected post-hoc comparison of means.

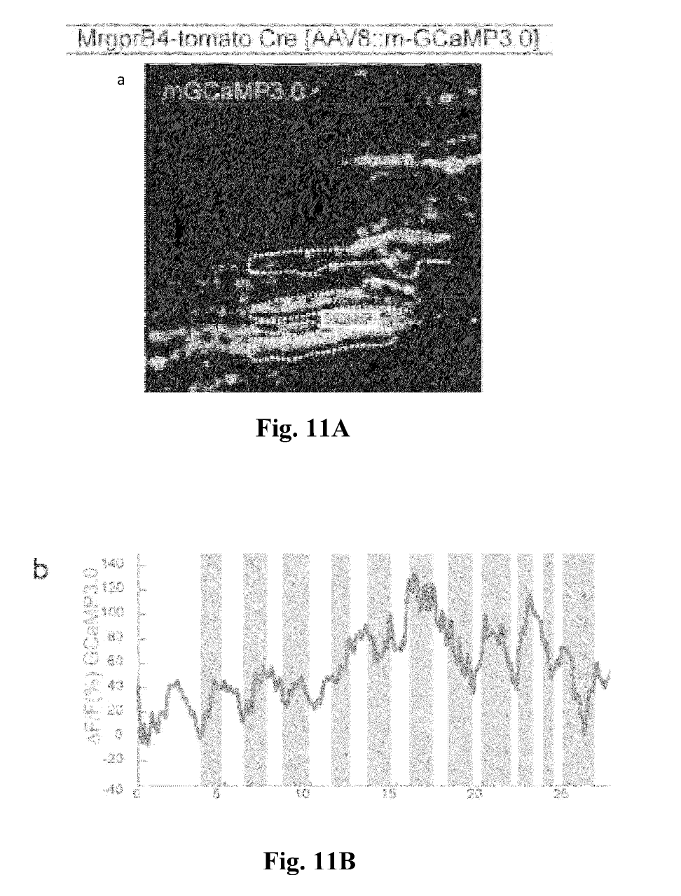

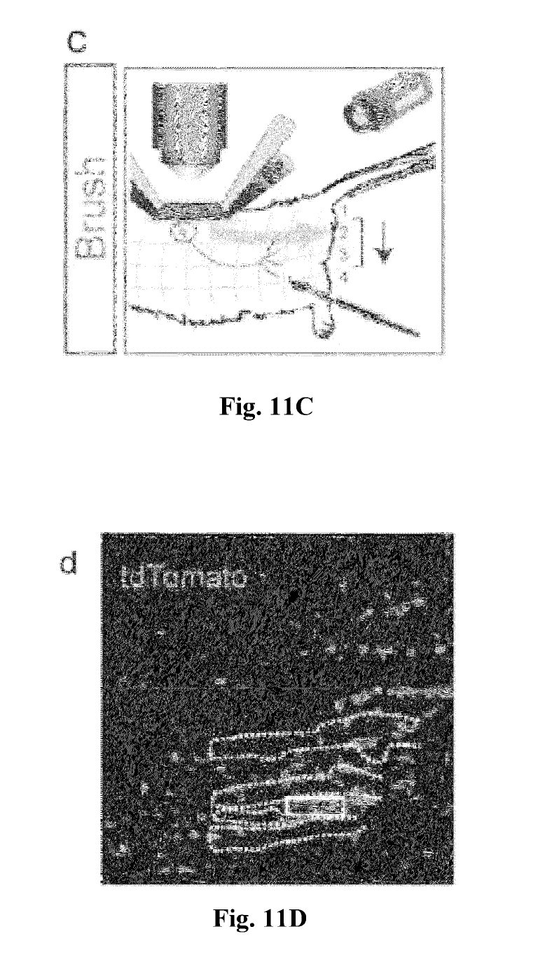

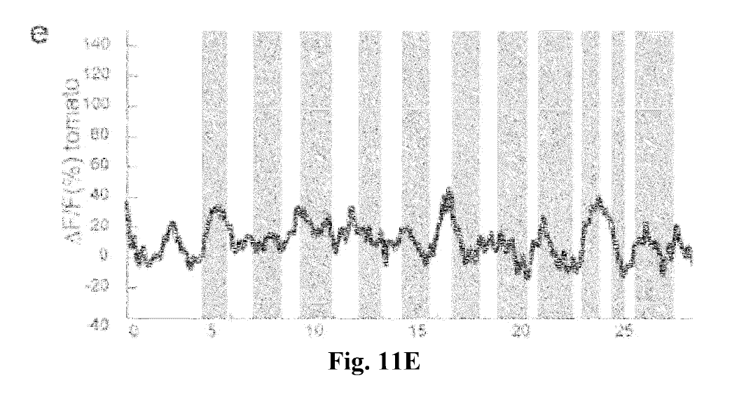

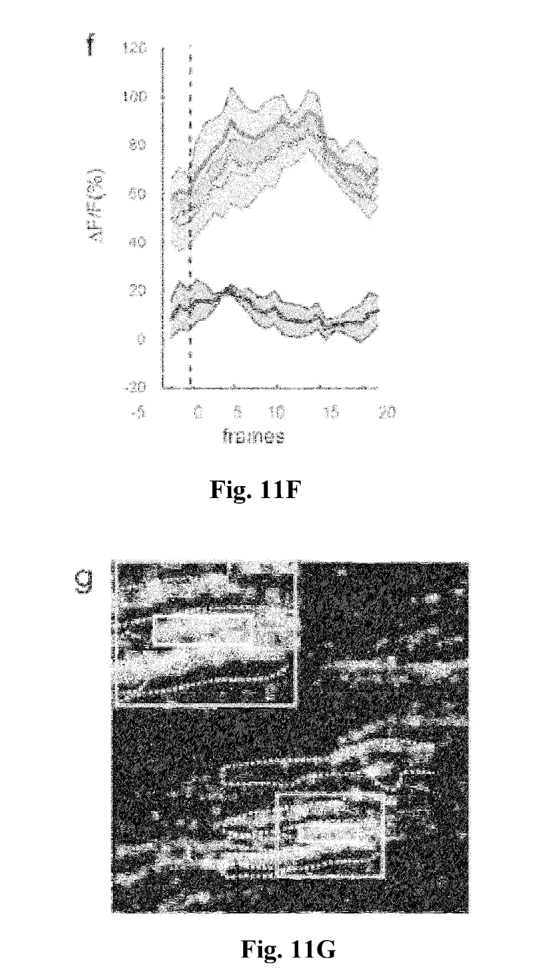

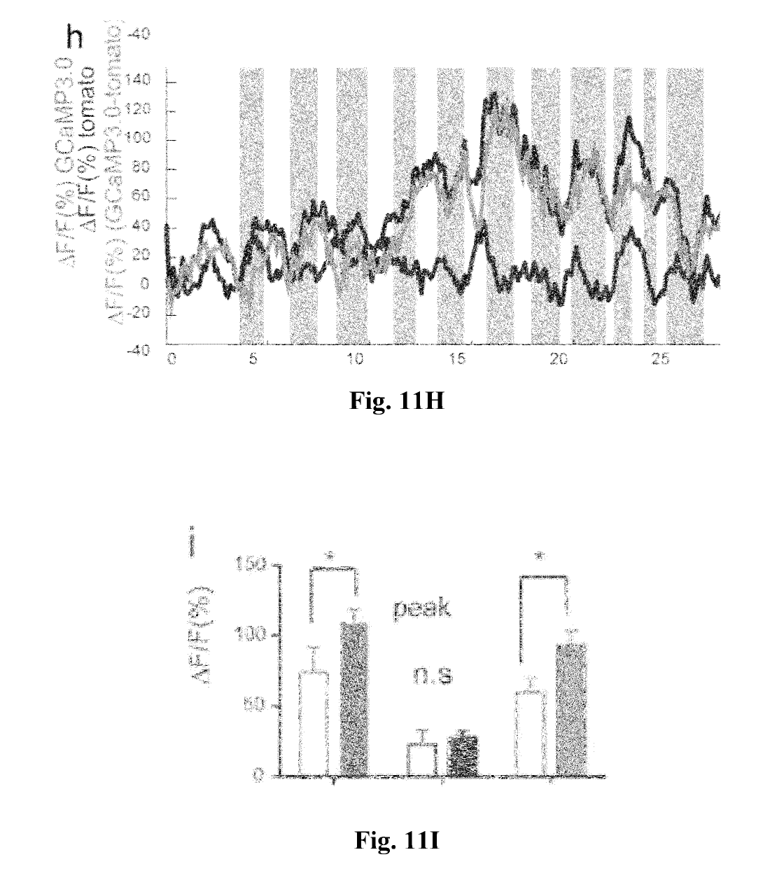

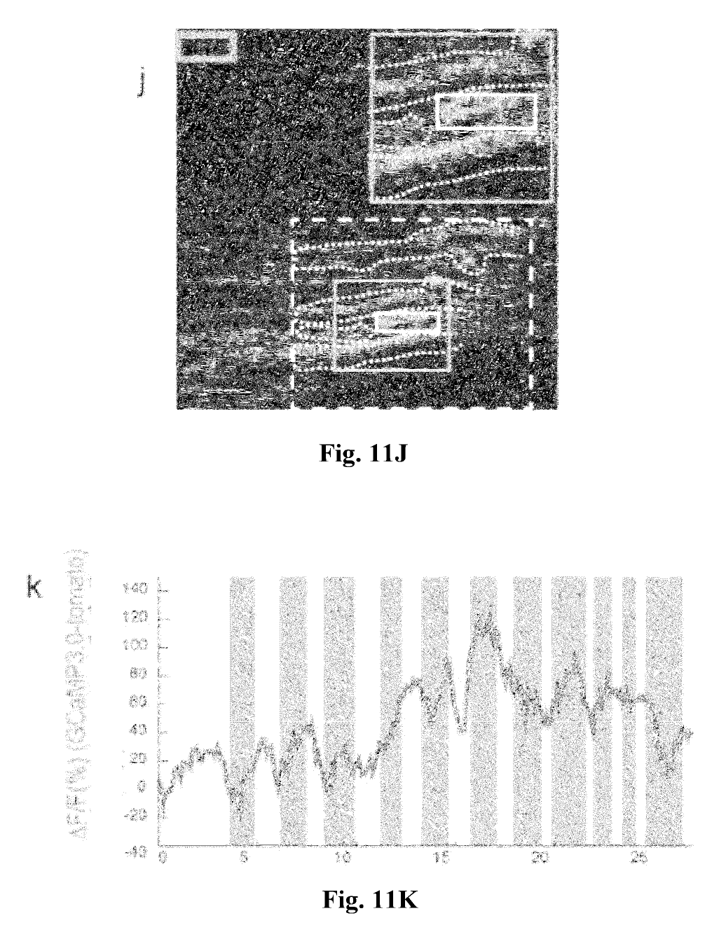

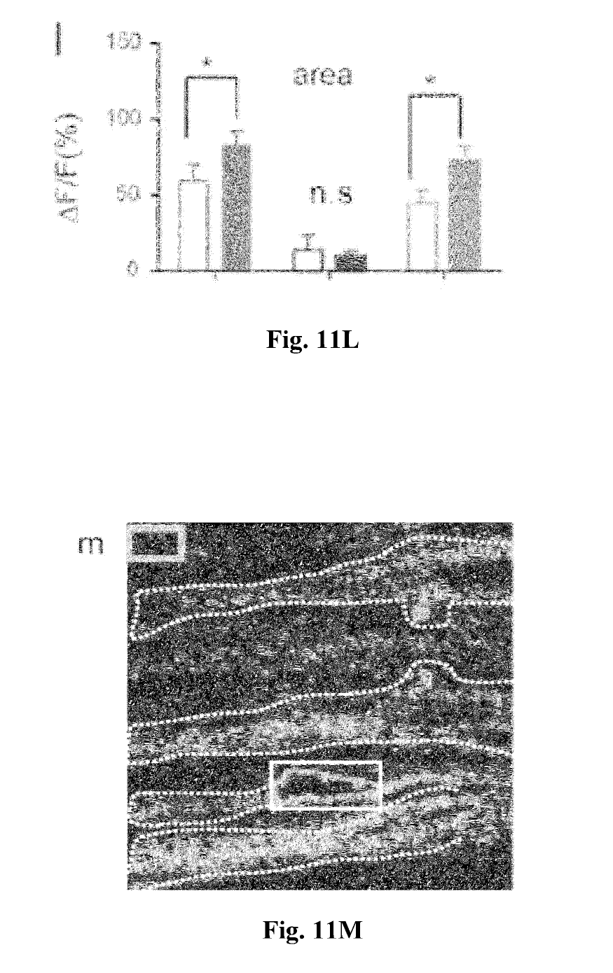

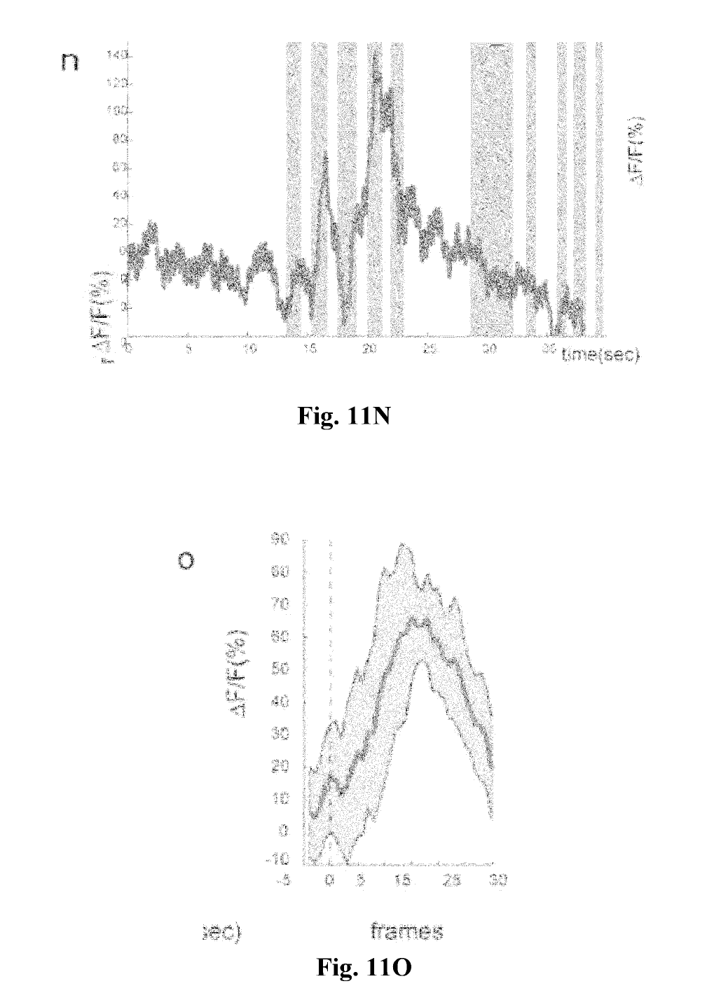

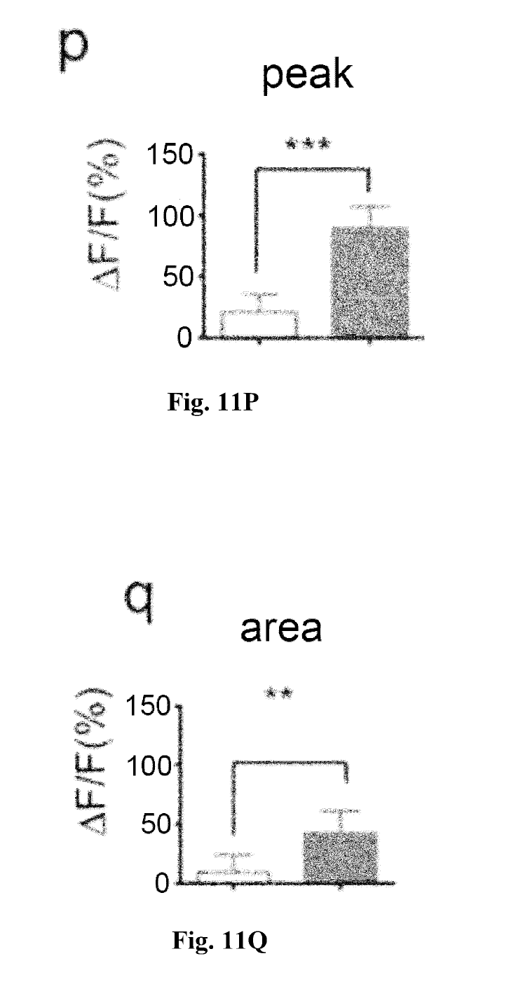

In the case of chemical stimulation (delivered either to the spinal cord or to the periphery), typically a single trial was performed for a given mouse, due to the difficulty of maintaining the same focal plane during the period of application of the chemical to the spinal cord or the period required for diffusion of the liquid bolus delivered for peripheral injection, respectively. In these cases, therefore, the MPI ΔF/Fₚₑₐₖ before and during the stimulation period were calculated for multiple mice imaged using ROIs of similar size, and were compared for statistical significance (relative to pre-stimulus baseline) by repeated measures ANOVA, followed by Bonferroni-corrected post-hoc comparison of means (unless stated otherwise). The ΔF/F values in FIGS. 11B and 11E were corrected for photobleaching as described in Berry, et al. Neuron 74:530-542 (2012).

### Behavior

The conditioned place preference (CPP) protocol was based on previous studies. For testing a positive valence effect of activation of MrgprB4⁺ neurons, a biased compartment assignment procedure in which activation of the neurons is tested for its ability was used to increase the time spent in the initially non-preferred chamber. The CPP apparatus consisted of a rectangular chamber divided into three compartments (300×150×150 mm per compartment), connected via an opening (50×50 mm) in each delimiting wall. The two side (test) compartments were designed to have different visual and tactile cues, by having distinct walls (horizontal or vertical alternating white and black stripes) and distinct floors (different shapes of floor grids with big or small square holes). In addition a 1-inch-diameter polyvinylchloride (PVC) pipe coupler (two schedule 40 wall thickness), either threaded or smooth, was placed in the centre of each side compartment to enrich for tactile cues. The centre compartment was a neutral plastic enclosure (see FIG. 4B). This design was chosen so as to promote a compartment preference assignment for each mouse. A video tracking system (Noldus Ethovision) recorded all animal movements.

Because our hypothesis is based on the social reward mediated by social contact in juvenile mice, the mice used were approximately 1-month old. After weaning the mice were maintained in social groups and left undisturbed until the start of the CPP assay. The paradigm was completed in 6 days. On the day before pre-testing the mice were socially isolated in their home cage. On day 1 of the procedure each mouse was placed in the central compartment and allowed to explore the entire apparatus freely for 30 min (pre-test). After the pre-test the initial preference of each mouse for a given side compartment was recorded. With our apparatus design most of the mice showed an initial preference for one of the two side compartments. Conditioning was initiated on day 2 and encompassed four sessions performed on four consecutive days. In the first conditioning session mice were injected i.p. with CNO (5 mg kg) (or saline of an equivalent volume for some control mice) and placed for 1 h (based on the observation that CNO effects peak between 45 and 50 min after administration) in the initially non-preferred (I.N.P.) compartment. On day 3, during the second conditioning session, all mice were injected with saline and confined for 1 hr in the opposite (that is, initially preferred, I.P.) compartment. (The second conditioning session was performed the following day as CNO effects last for 9 hrs.) On day 4 and day 5 the first and second conditioning sessions were repeated, respectively. The time between the i.p. injections of CNO or saline and the placement of the mice in the compartment was between 5-10 min, which is compatible with the time that is needed for CNO to start having an effect. On day 6, the mice were tested for their side compartment preference by placing them in the centre compartment and allowing them to explore the entire apparatus freely for 30 min (post test). All sessions were conducted blind to the genotype/injected virus of each mouse. For the conditioned place aversion (CPA assay) the mice remained group-housed until the day before the pre-test. After the pre-test, on the first day of the conditioning session the mice were injected with saline and confined in the I.N.P. compartment. On the second day of conditioning the mice were injected with CNO (except for the saline control mice) and placed in the I.P. compartment. On the third and fourth day of conditioning the first and second sessions of conditioning were repeated, respectively. On the sixth day the mice were tested for their preference in the three-compartment arena.

### Behavioral Data Analysis

Difference scores for each chamber (time in chamber during post-test minus time in chamber during pre-test) were analyzed for statistical significance (significant difference from zero) using simple or repeated one-way ANOVA (P<0.05) followed by a Bonferroni-corrected post-hoc comparison of means. For the comparison of the mean difference scores between the experimental and the pooled control groups, as depicted for example in FIG. 4J, an unpaired t-test was used. Statistical analysis of all other metrics was performed using a repeated two-way mixed model ANOVA (unless otherwise stated) (with each group as the between—subject variable and pre-training versus post-training as the within-subject variable). Detection of a significant interaction and/or main effect was followed by Bonferroni-corrected post-hoc comparison of means.

### Drugs

Clozapine-N-oxide (CNO) was obtained from Biomol International, and dissolved in saline.

### Example 1

### Detection of MrgprB4⁺ Neurons Activation in Isolated Skin-Nerve Preparations

This example shows that in isolated skin-nerve preparations, MRGPRB4 neurons were not electrophysiologically activated by mechanical, thermal or chemical stimuli.

MrgprB4⁺ neurons are distinct from a recently characterized population of tyrosine hydroxylase (TH)-positive C-fiber low-threshold mechanoreceptors (C-LTMRs), which do not express Mrgprs or bind IB49. In isolated skin-nerve preparations, MrgprB4-expressing neurons (identified using an EGFP reporter; see methods described above) responded neither to punctate stimulation using von Frey filaments (1-200 mN force), nor to gentle stroking with a paintbrush (0/25 neurons tested; see methods described above), nor did they respond to thermal or to a cocktail of chemical stimuli. By contrast, MrgprD-expressing neurons were activated by von Frey filaments by forces up to 100 mN in such preparations, consistent with a requirement of these neurons for normal behavioral responses to noxious mechanical stimulation in vivo.

### Example 2

### Preparation of MrgprB4-tdTomato-2A-Cre Mice

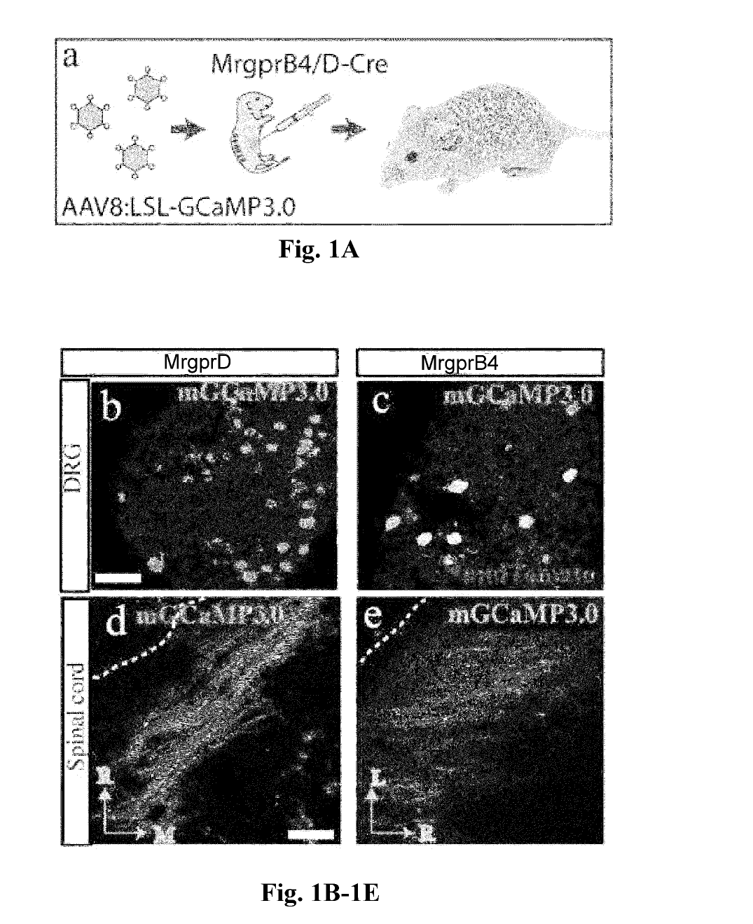

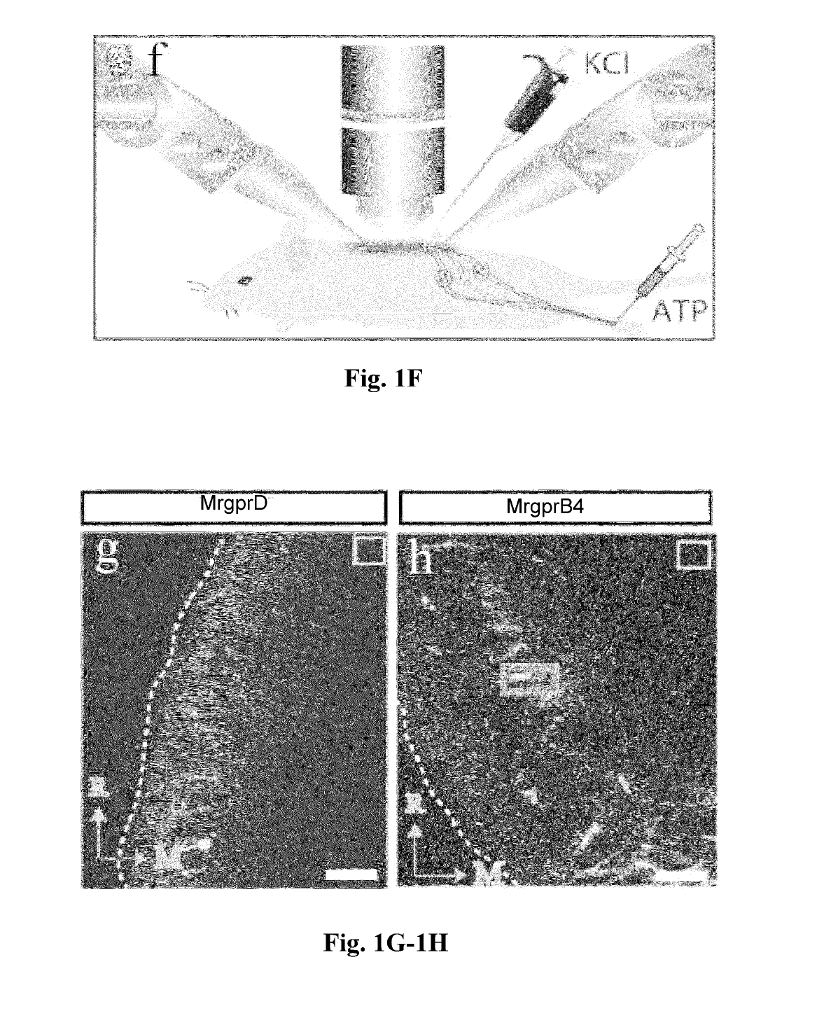

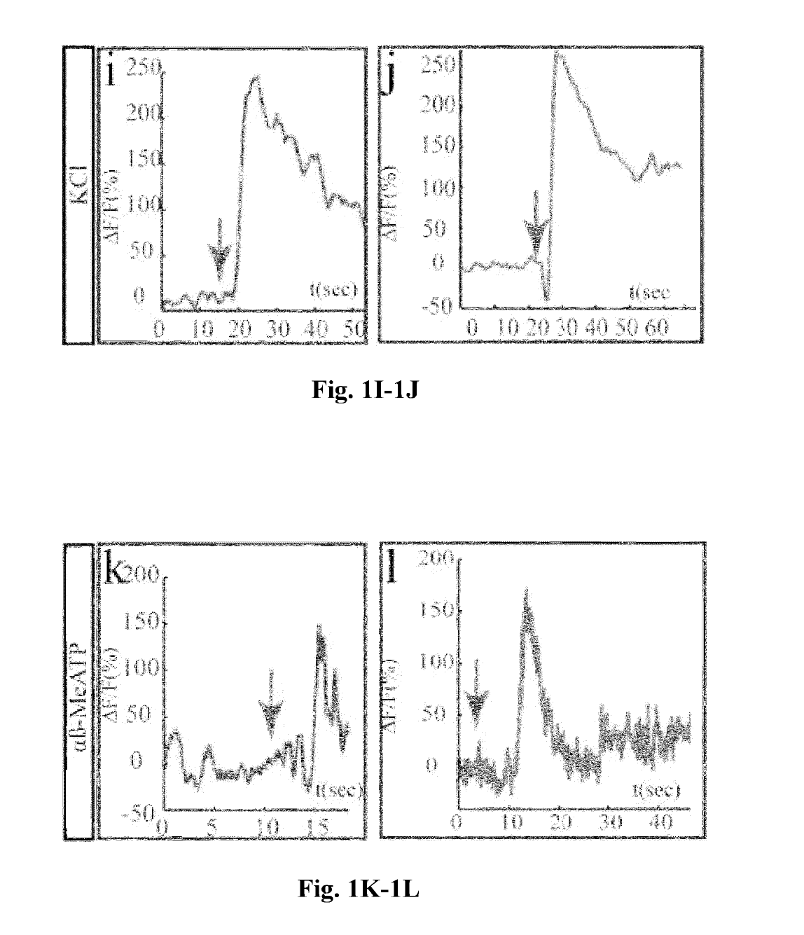

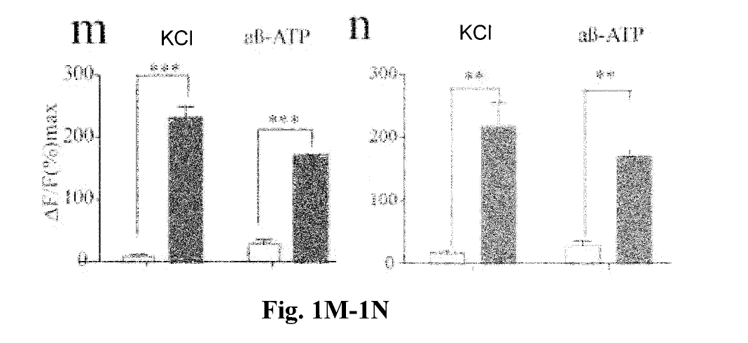

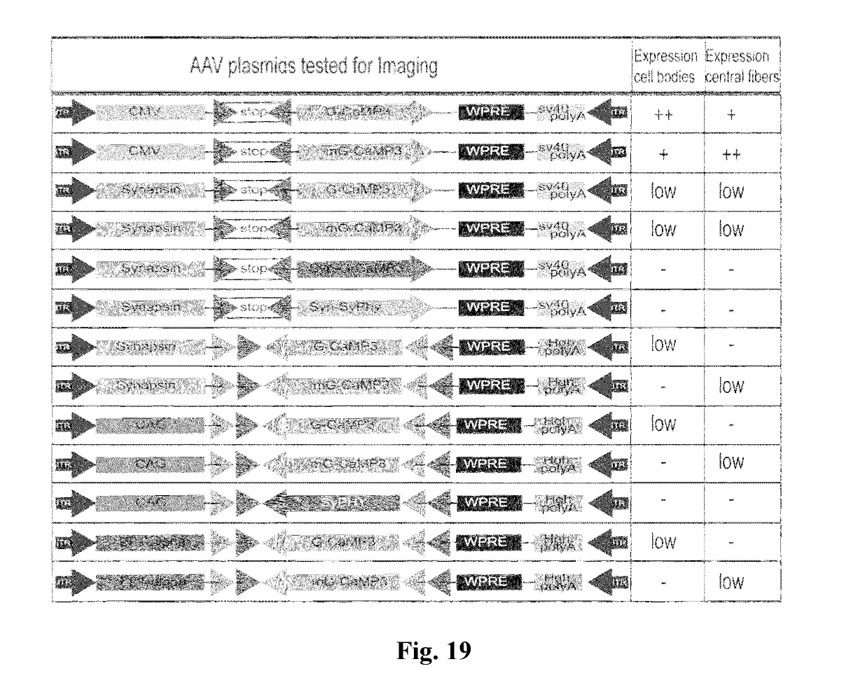

A potential drawback of introducing exogenous genes into primary cutaneous sensory neurons in vivo by viral transduction is that infection of these neurons typically requires injection into the periphery, sciatic nerve or dorsal root ganglia (DRG). This yields a highly localized distribution of infected cells whose peripheral receptive fields may be equally restricted, conflating the problems of stimulus identification and receptive field localization. To circumvent this, we confirmed and extended a report suggesting that intra-peritoneal (i.p.) injection of adeno-associated virus of serotype 8 (AAV8) into neonatal pups results in widespread infection of DRG neurons in adults (Foust et al., Hum. Gene. Ther. 19:61-70 (2008)). I.p. injection of P0-P2 MrgprB4-tdTomato-2A-Cre mouse pups with a Cre-dependent AAV8 expressing either cytoplasmic or membrane-tethered forms of GCaMP3.0 (mGCaMP3.0; FIG. 19) under the control of the cytomegalovirus (CMV) promoter indeed yielded effective expression of the virally encoded GECI. Expression in MrgprB4⁺ neurons was both relatively efficient ([viral GCaMP3.0+, tdTomato+/tdTomato+]=0.62±0.06; mean±SEM, n=24 sections), and specific in that the majority of GCaMP3.0+ cells were tdTomato+([viral GCaMP3.0+, tdTomato+/GCaMP3.0+]=0.62±0.05). The incomplete overlap likely reflects variable levels of MrgprB4-tdTomato-2A-Cre expression and the fact that low levels of Cre (and therefore perhaps undetectable levels of tdTomato) can lead to recombination. A similar level of specificity was observed in MrgprD-EGFPCre mice infected with a Cre-dependent hrGFP AAV (see FIG. 1a, b, d and Suppl. FIG. 2a-c,g; [viral hrGFP+, EGFPCre+/hrGFP+]=0.62±0.036, n=9)

To target genetically encoded calcium sensors to MRGPRB4 or MRGPRD neurons, neonatal Mrgprb4-tdTomato-2A-cre mice (FIG. 5) or MrgprD-EGFP-cre mice were injected intraperitoneally (i.p.) with a Cre-dependent adeno-associated virus (AAV) expressing GCaMP3.0 as described in Tian et al., Nature methods, 6:875-881 (2009) (FIG. 19). A similar efficiency of viral expression (62±3.6%) was observed in MrgprD-EGFP-cre mice (FIG. 1b, Supplementary FIG. 2a-c, g). This approach yielded relatively efficient expression of the genetically encoded calcium sensor in MRGPRB4::tdTomato1 dorsal root ganglia (DRGs) neurons (62±6%) along the rostro-caudal axis in adult mice (FIG. 1a, c, Supplementary FIG. 2d-f, h). Expression of GCaMP3.0 or mGCaMP3.0 was especially robust in the central spinal projections of these neurons (FIG. 1d, e). No expression of the reporter was observed in virally injected wild-type mice.

### Example 3

### Two-Proton Imaging of Calcium Transients in MrgprD⁺ and MrgprB4⁺ Neurons

In this example, calcium imaging was performed specifically in MrgprB4⁺ neurons while stimulating the periphery of intact mice.

To record calcium transients in the central projections of MRGPRD+ or MRGPRB4+ neurons, two-photon imaging was performed through a spinal cord laminectomy while stimulating the intact animal (FIG. 1f). Anaesthetized mice were mounted under a two-photon microscope (Prarie Instruments, Inc.) in a suspension system designed to minimize breathing-associated movement artifacts, and imaged ˜100-250 μm below the pia through an agarose-covered dorsal laminectomy covering two lumbar segments (L1-L3 or L2-L4) (FIG. 1f). Similar results were obtained using either a membrane-tethered form of GCaMP3.0 (mGCaMP3.0) or a cytoplasmic form of the GECI (FIG. 19).

Responses to centrally or peripherally applied chemical stimuli were tested first. Direct application to the spinal cord of depolarizing concentrations of KCl elicited robust fluorescence increases over baseline, ΔF/F, in both MRGPRD⁺ fibres (FIG. 1g, i, m; mean percent increase in peak ΔF/F (MPI 4F/Fₚₑₐₖ)=222±19% (±s.e.m.); mean latency to peak (MLP)=8.6±3.6 s, n=3) and MRGPRB4⁺ fibres (FIG. 1h, j, n; MPI ΔF/Fₚₑₐₖ=201.6±33.2%, MLP=9.3±4.15 s, n=3).

We also observed responses to α,β-methylene (Me) ATP, a ligand known to activate both MRGPRB4⁺ and MRGPRD⁺ neurons in vitro, via both direct spinal application and/or peripheral injection into hairy or glabrous skin of the hindpaw, respectively (FIG. 1k-n and Supplementary FIG. 3). Calcium responses in MrgprD⁺ neurons were activated by spinal application of α,β Me-ATP (Supplementary FIG. 3, MPI [ΔF/F]ₚₑₐₖ=249±54%, MLP=16.3±1.3, mean±range, n=2), consistent with previous studies indicating that these neurons are ATP-responsive. Calcium transients were evoked in MrgprD⁺ fibers by injection of α,β Me-ATP into the glabrous skin of the hind paw (FIG. 1k, m; MPI [ΔF/F]ₚₑₐₖ=137±51%, MLP=25.7±10.5 sec, n=3) and in MrgprB4⁺ fibers by injection of α,β Me-ATP into the dorsal (hairy) skin of the hindpaw (FIG. 11, n; MPI [ΔF/F]ₚₑₐₖ=142±0.6%, MLP=21.9±12.6 sec, n=3).

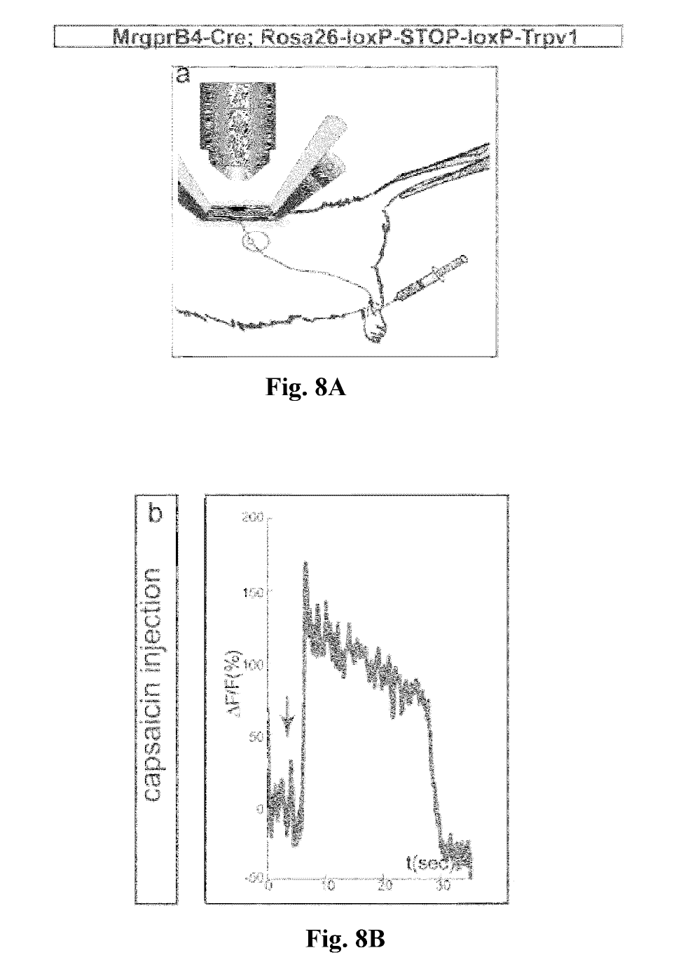

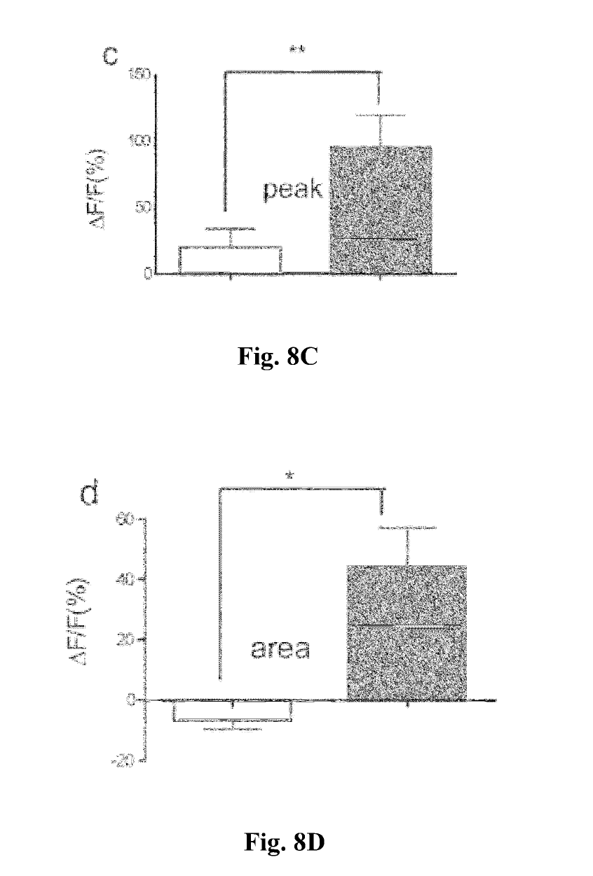

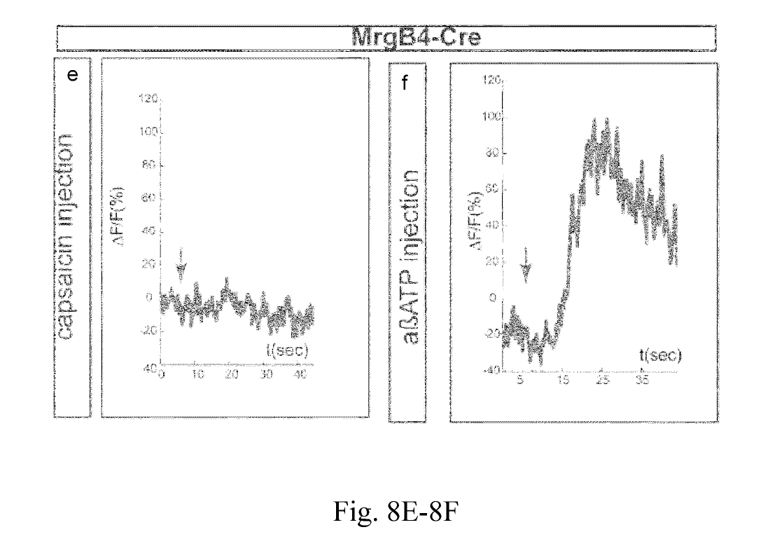

As an additional validation of the ability to image activation of MrgprB4-expressing fibers by a peripherally injected specific ligand, TrpV1 was mis-expressed in these fibers (which normally do not express this channel) by crossing MrgprB4-Cre mice to Rosa26-loxP-STOP-loxP-TrpV1 mice, and injected them neonatally with Cre-dependent AAV encoding GCaMP3.0. Peripheral injection of adult MrgprB4-Cre; Rosa26-loxP-STOP-loxP-TrpV1 mice with capsaicin induced robust calcium transients, while no such signals were observed in control MrgprB4-Cre mice injected with capsaicin (FIG. 8). It was found that MRGPRB4⁺ central fibres were activated by peripheral injection of capsaicin in mice genetically engineered to express TRPV1 in MRGPRB4⁺ neurons, which normally do not express this channel (FIG. 8). Therefore, the preparation disclosed herein was able to detect calcium transients in both MRGPRD⁺ and MRGPRB4⁺ fibres by peripheral injection of specific chemical stimuli that activate these neurons.

### Example 4

### Imaging Activity Evoked by Mechanical Stimulation of the Periphery

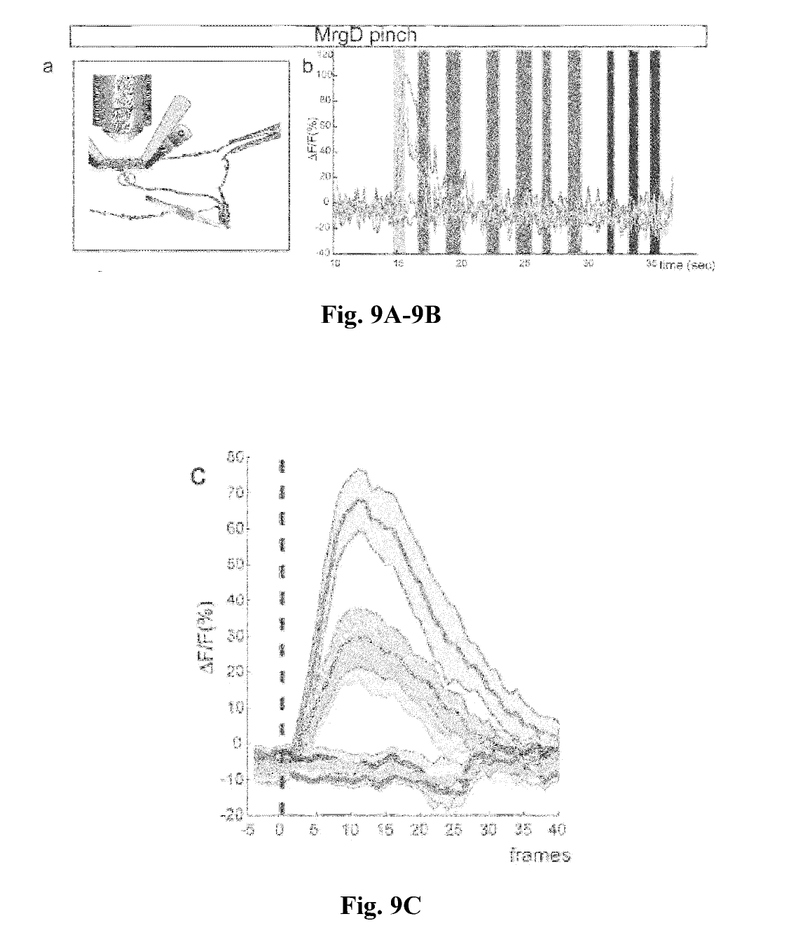

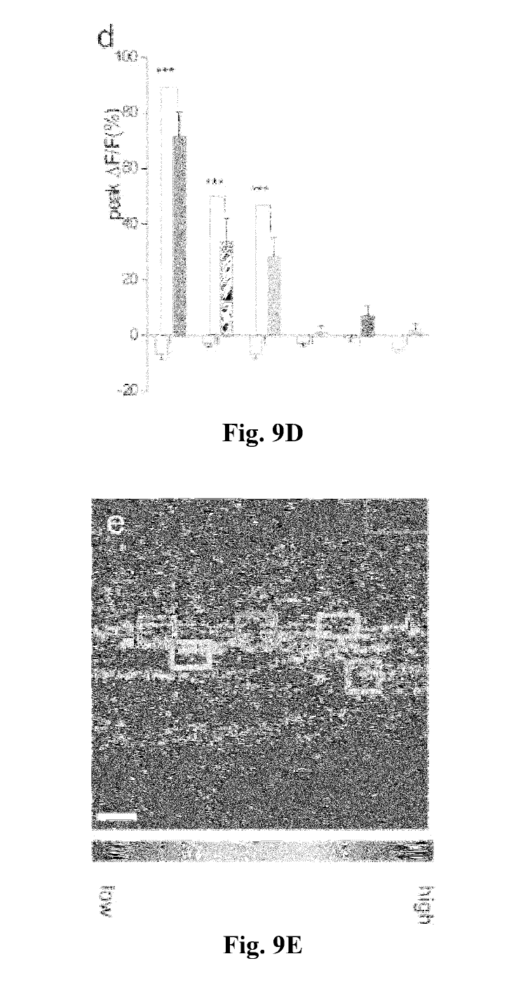

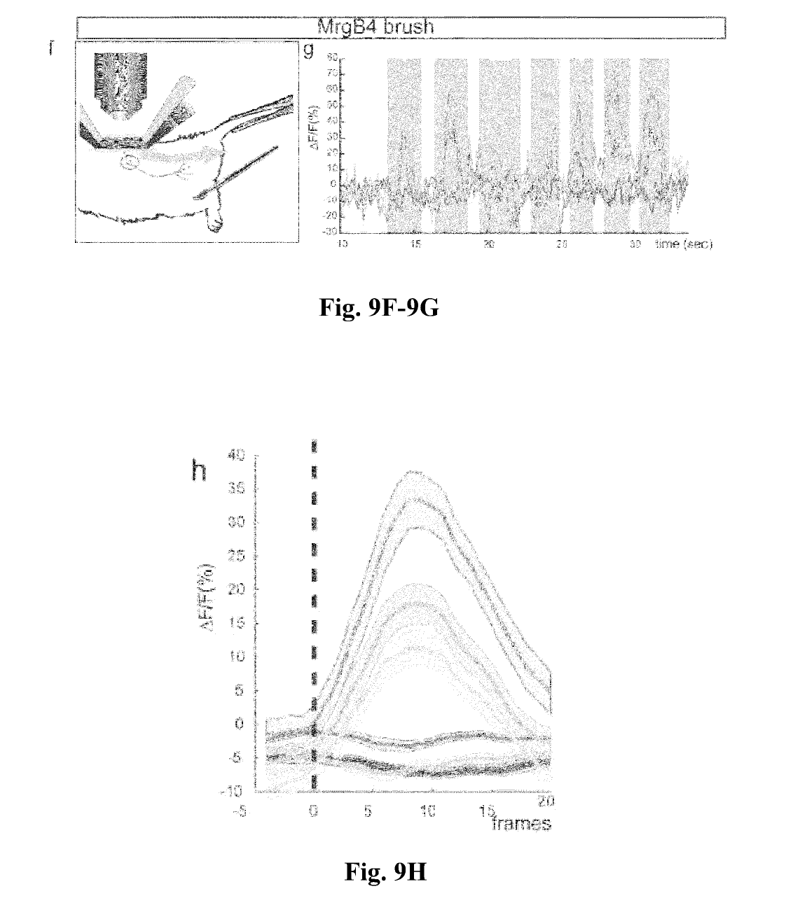

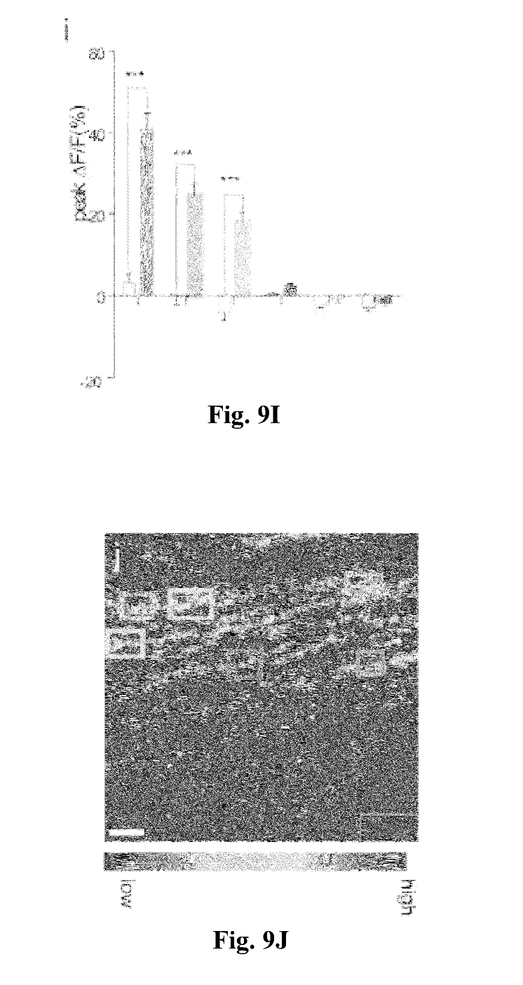

The preparation described in Example 2 was tested for imaging activity evoked by mechanical stimulation of the periphery. Activity in MRGPRD⁺ fibres after mechanical stimulation of the hindpaw was measured using a custom pinching device (FIG. 2a). In agreement with their established role in sensing noxious punctate mechanical stimuli, MRGPRD⁺ fibres were strongly activated by trains of pinching stimuli in the ipsilateral hindpaw (and more specifically in the particular experiment by pinching of the most distal ipsilateral digit) (FIG. 2d, e; MPI ΔF/Fₚₑₐₖ=77.8±8.9%, n=7 trials per mouse). Responses were restricted to a subset of GCaMP3.0-expressing fibres within a given imaging field, whereas other fibres were unresponsive (FIG. 2c-e, h and FIG. 9a-e). This heterogeneity probably reflects the different receptive fields of these fibres relative to the site of stimulation.

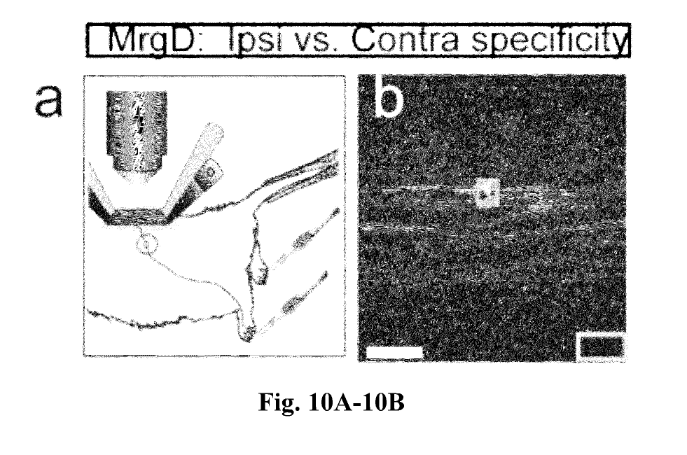

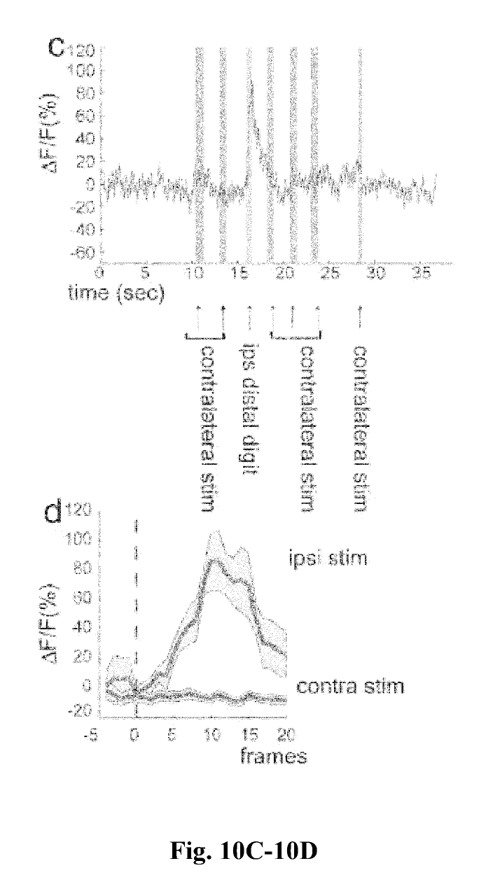

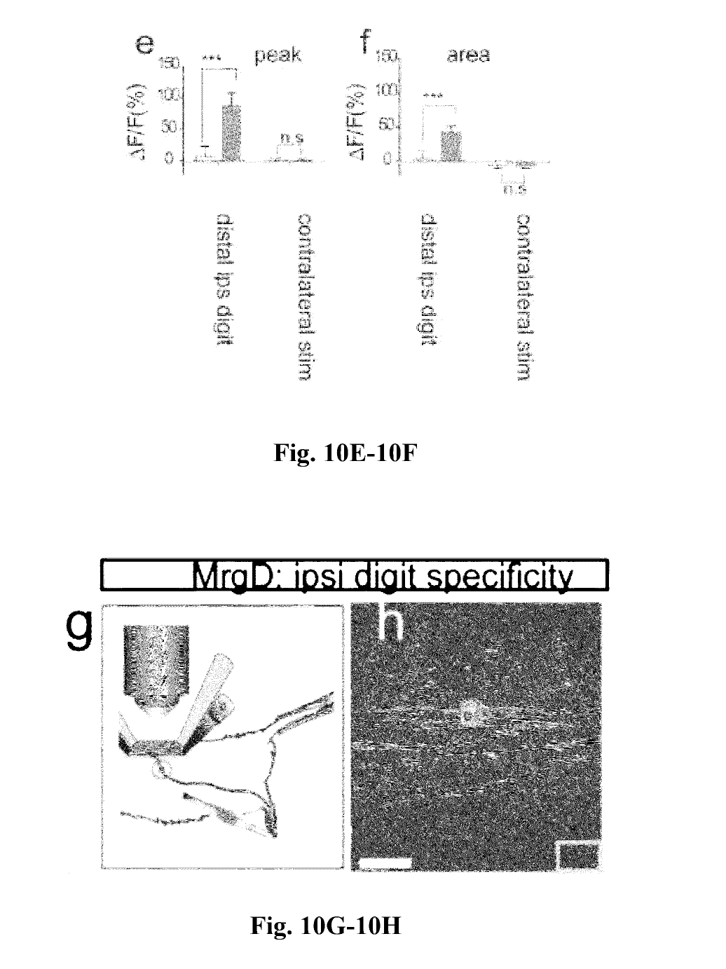

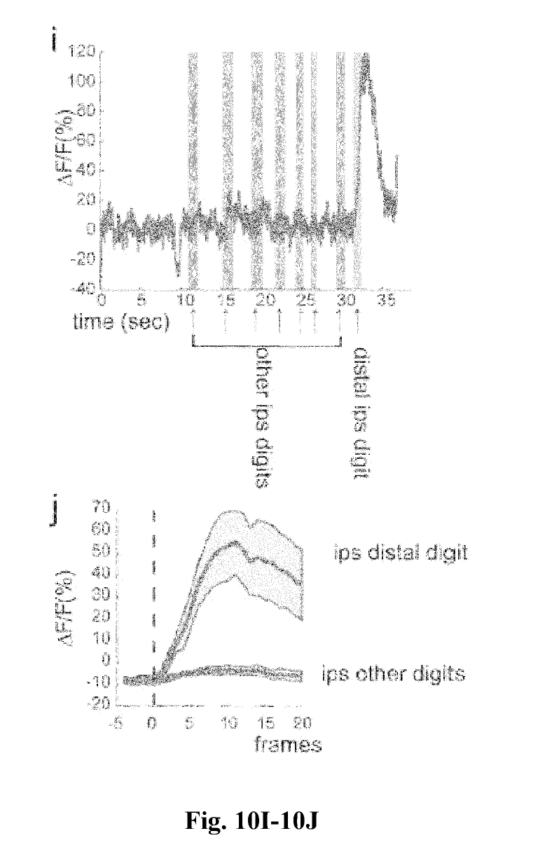

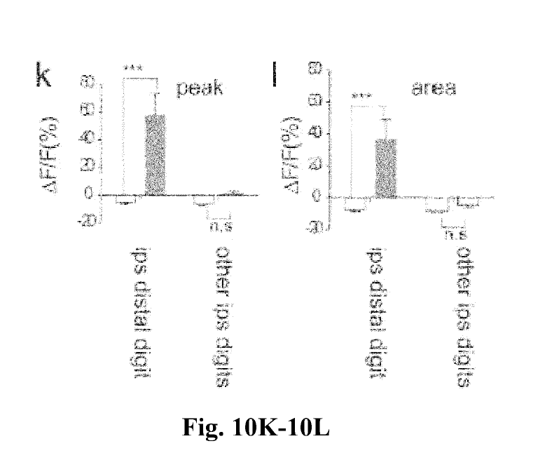

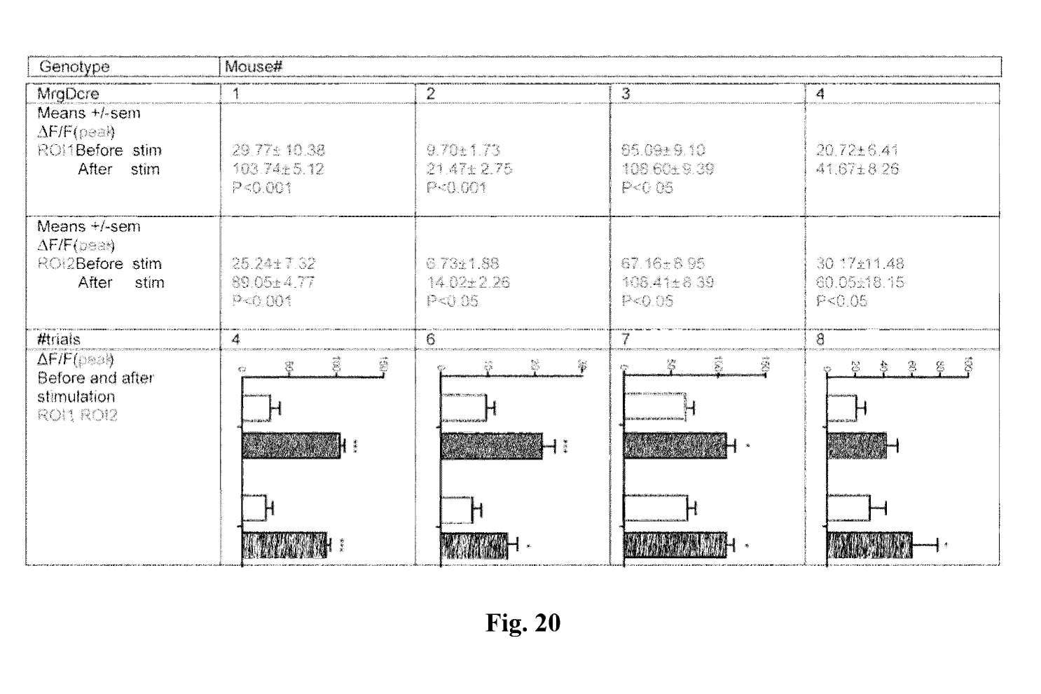

In MrgprD::GCaMP3.0 mice, pinching of the contralateral hind paw evoked no responses in MrgprD⁺ fibers activated by stimulation of the ipsilateral paw (FIG. 10a-f). Moreover, Ca⁺ transients in a specific ROI were evoked only when pinching was applied to a particular digit of the ipsilateral hindpaw, and not to other digits (FIG. 10g-l), suggesting that the responses were specific to a given peripheral receptive field. The ΔF/F responses for a given ROI were reproducible across trains of stimuli within a trial (FIG. 2d), as well as across multiple trials in a given mouse (FIG. 2e, h), and were independently observed in 5 different mice (MPI [ΔF/F]ₚₑₐₖ=47.4±12.7%, n=5 mice; FIG. 2j and FIG. 20). Therefore, responses of MRGPRD⁺ fibres to pinching were reproducible across trials and mice (FIG. 2d, e, h, j and FIG. 20), and also specific to the ipsilateral hindpaw and to particular digits (FIG. 10).

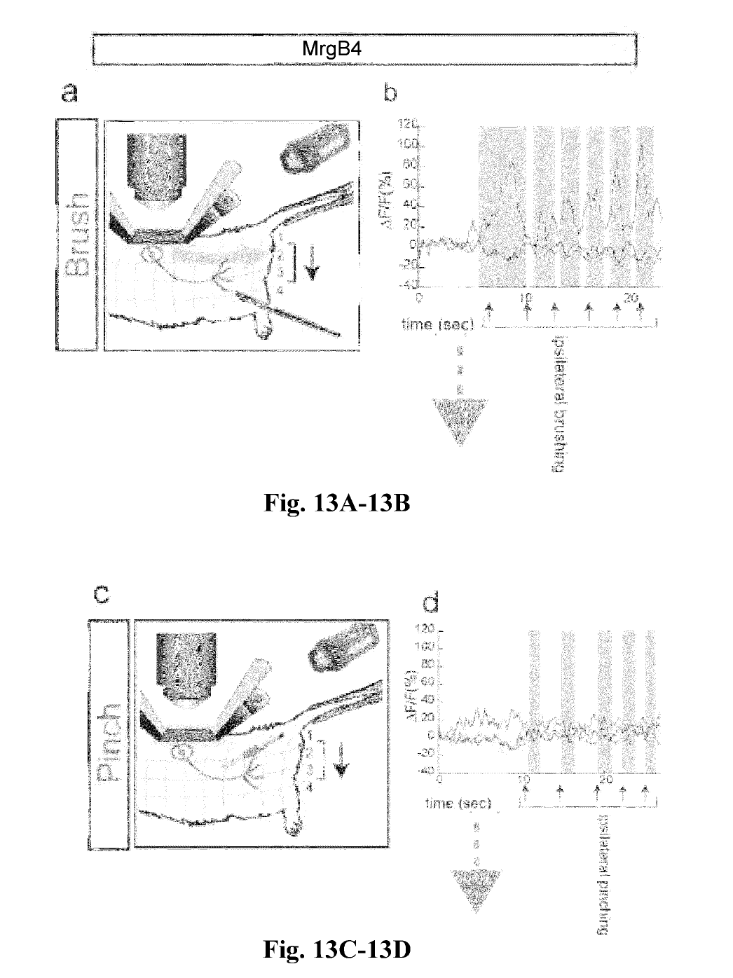

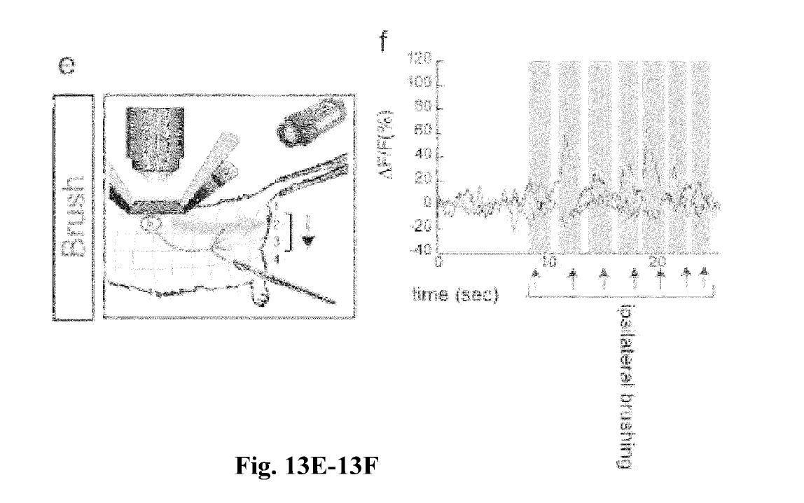

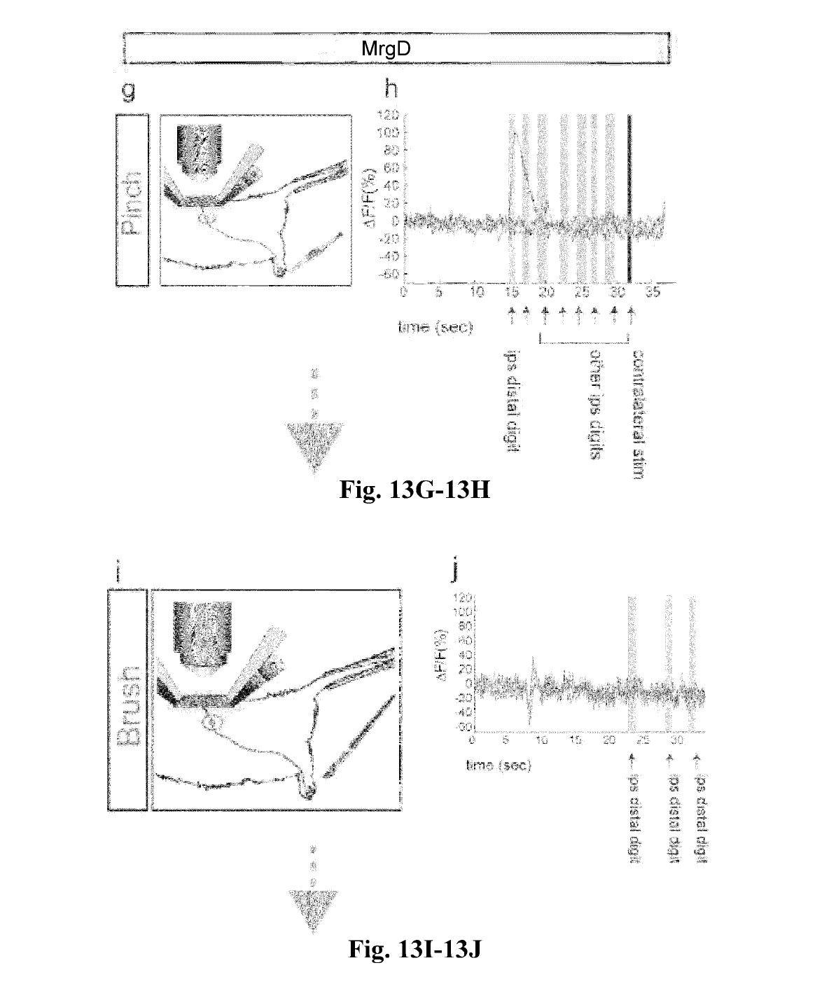

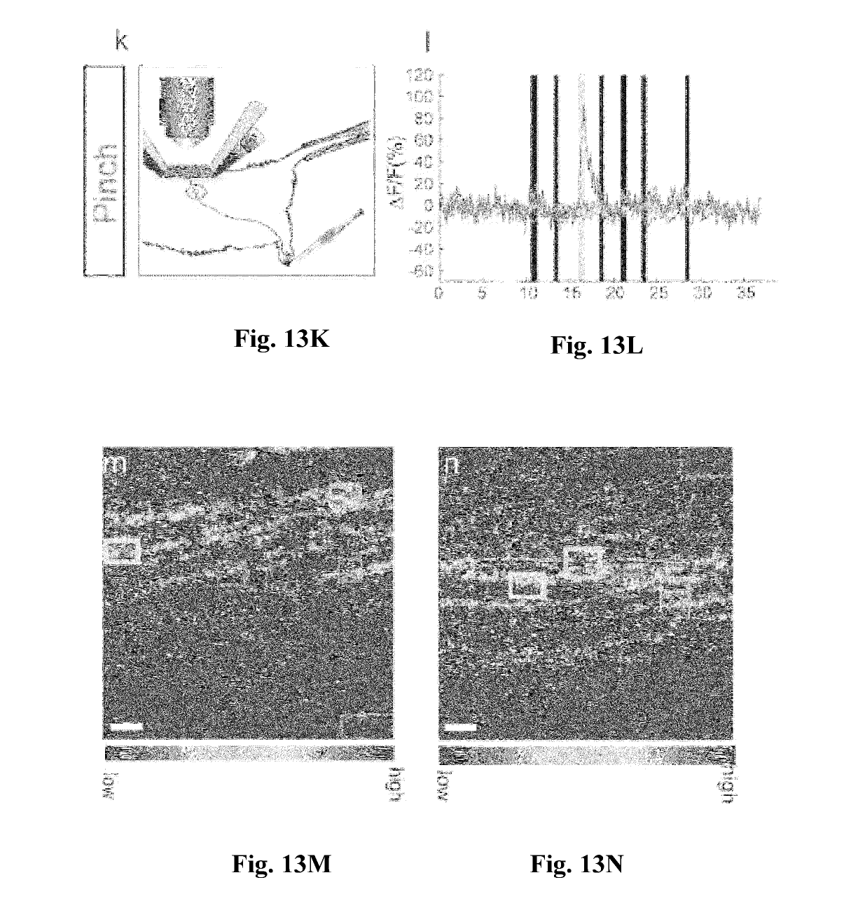

It was noted that MRGPRD⁺ fibres in a given region of interest (ROI) that were activated by pinching were not activated when the last digit of the ipsilateral hindpaw was stroked lightly using a brush (FIG. 2f, g, i). The same fibres could, however, be reactivated by a subsequent pinching stimulus (FIG. 13i-l), indicating that the lack of response to brushing was not due to adaptation or desensitization produced by the pinch stimulus. These data therefore suggest a specificity of MRGPRD⁺ fibres for punctate or focal noxious mechanical stimulation of the skin.

### Example 5

### Functional Characterization of MrgprB4⁺ Fibres

To functionally characterize MRGPRB4⁺ fibres, a variety of innocuous mechanical stimuli designed to simulate natural stroking or grooming were tested, using a custom-designed brush (FIG. 3a, FIG. 18). Calcium transients in MRGPRB4-tdTomato⁺ fibres (FIG. 11) were elicited by repeated stroking (0.2-0.5 Hz) of relatively large areas (2-3 mm×20-30 mm) of posterior dorsal thoracic and proximal hindlimb hairy skin (FIG. 3c-e, h; green traces), consistent with the distribution of MRGPRB4⁺ fibres in the periphery. The average forces and velocities delivered from these manual stimuli, which included a mild pressure component, were relatively dynamic but fell within the range of 20-90 mN and a speed of 0.5-2 cm/s.

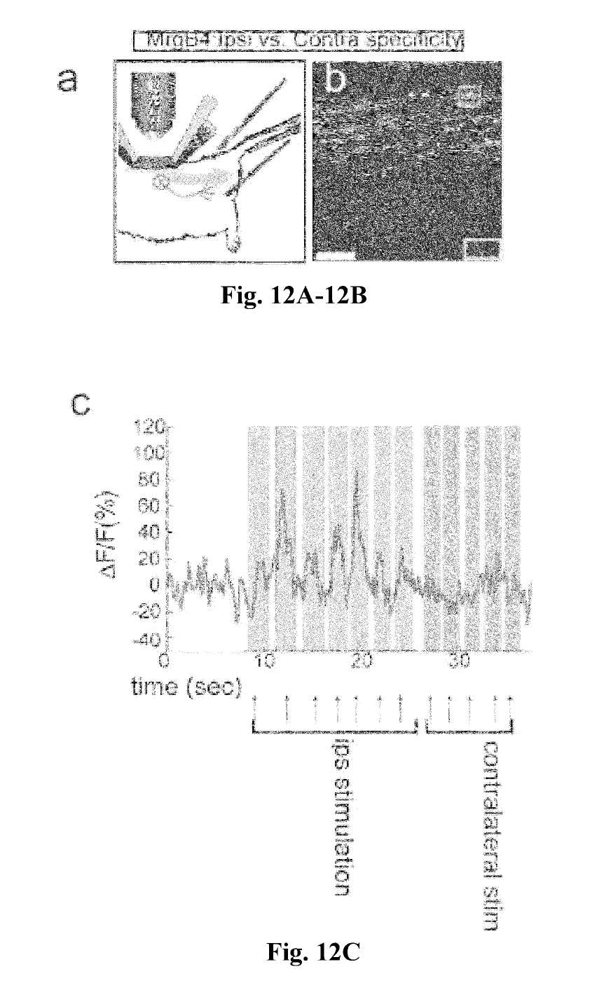

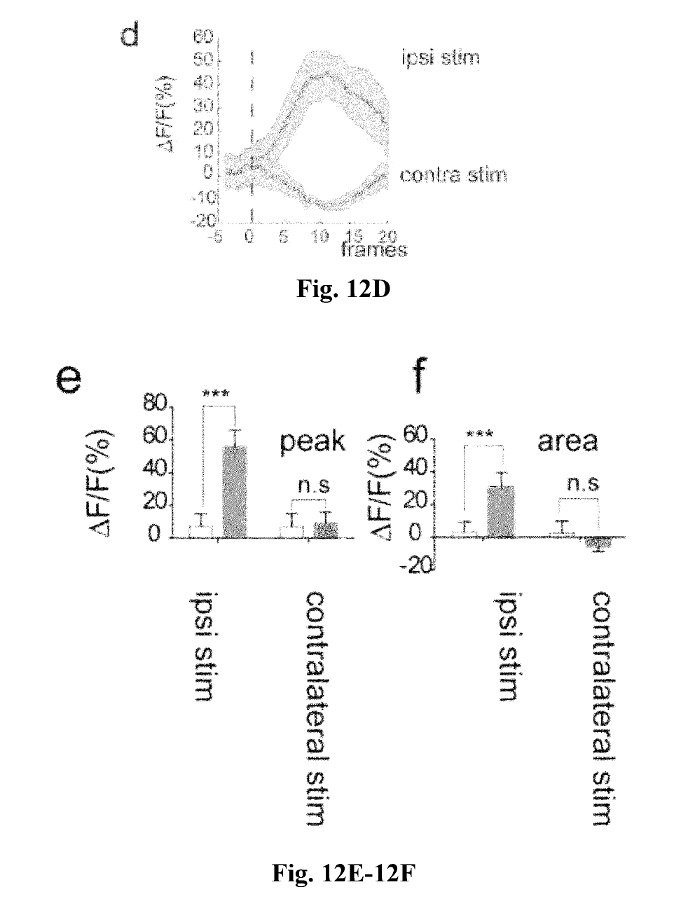

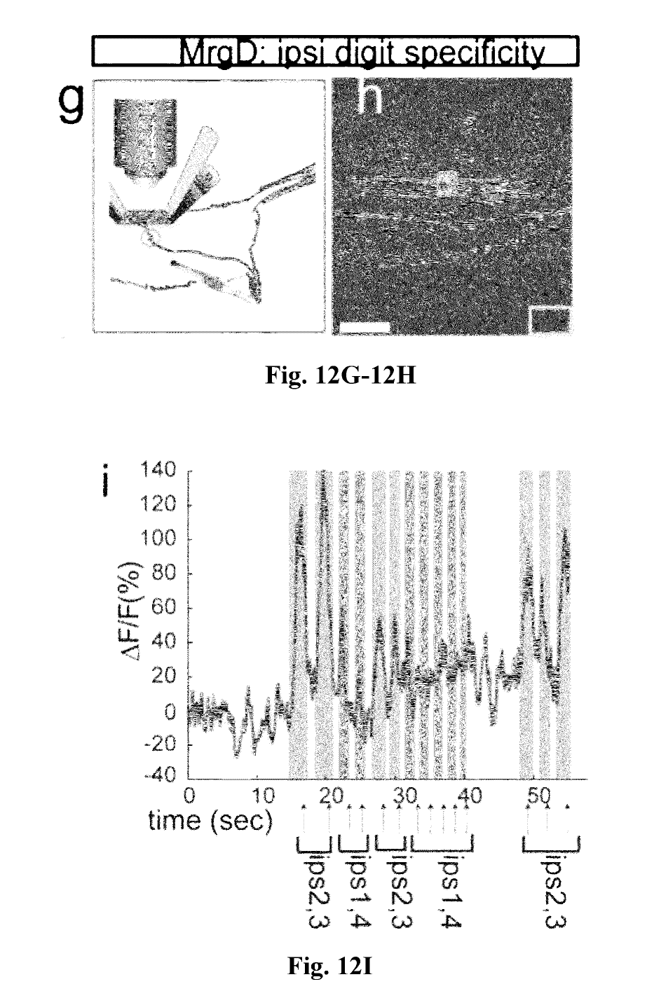

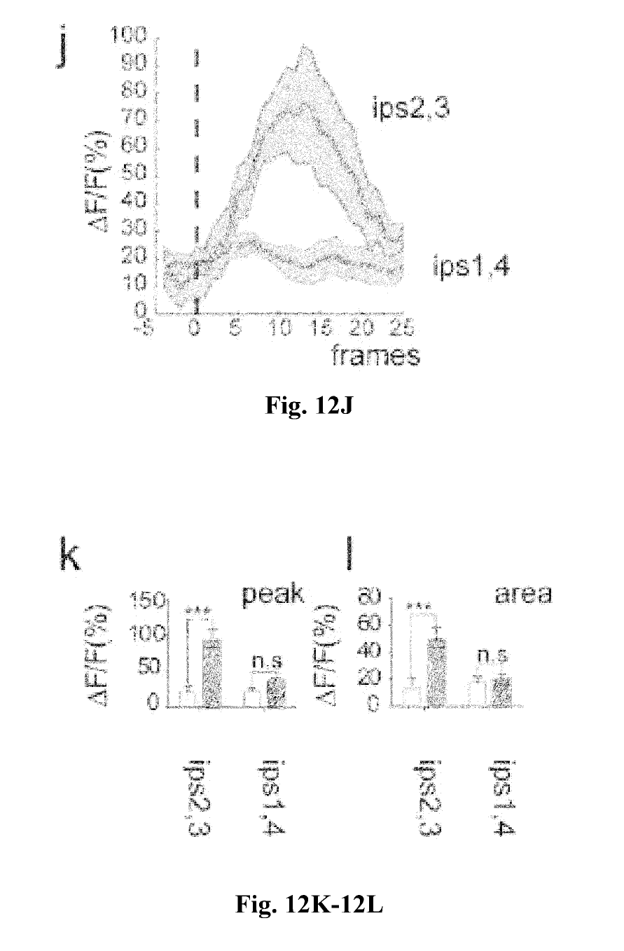

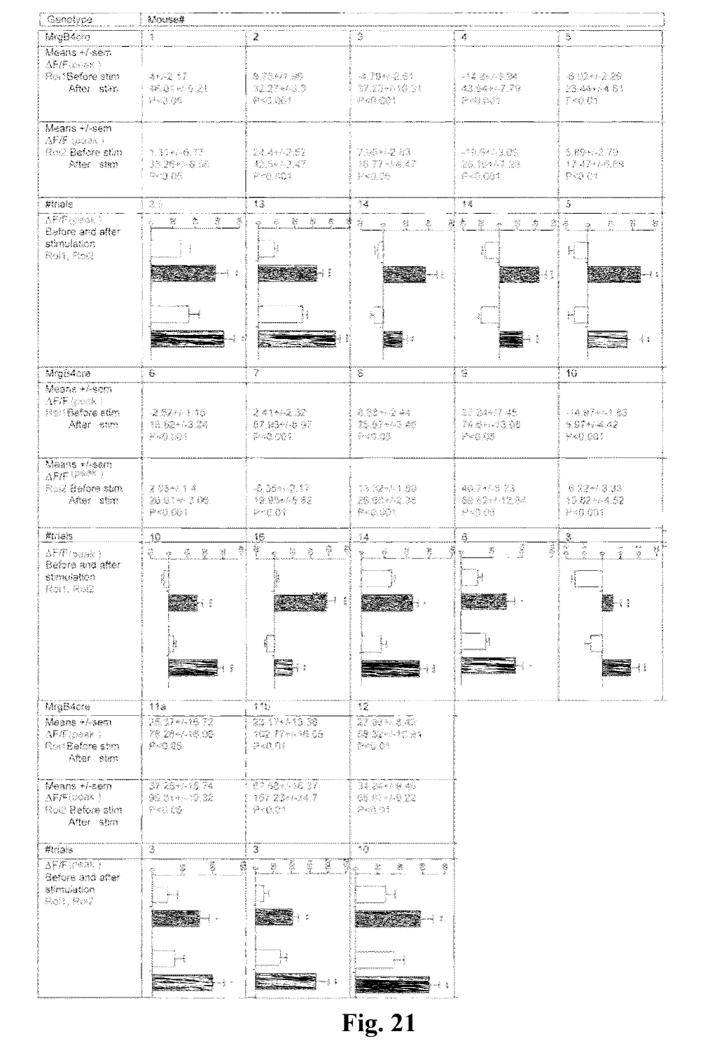

As in the case of the MrgprD⁺ fibers, Ca⁺ transients elicited in MrgprB4⁺ fibers by stroking the skin were specific for a particular ROI in a given field of view (FIG. 3c-d and FIG. 9g-i), occurred synchronously with the delivery of stimulation (FIG. 3d, light blue bars), and were evoked by stimulating the ipsilateral but not the contralateral side of the animal (FIG. 12a-f). Moreover, Ca⁺ transients were evoked by stroking certain regions of the skin (identified by projecting a light grid onto the mouse), but not by stroking other, neighboring regions (FIG. 12g-l). Although the responses were somewhat variable in magnitude, they were reproducible across trains of stimuli in a given trial (FIG. 3d and FIG. 11), multiple trials in a given mouse (FIG. 3e, h, green bars; MPI [ΔF/F]ₚₑₐₖ=38.8±4%, n=5 trials), and were observed in multiple animals (MPI [ΔF/F]ₚₑₐₖ=42.5±5.8%, mean±SEM, n=13 mice; FIG. 3j and FIG. 21).

In contrast to MRGPRD⁺ fibres, MRGPRB4⁺ fibres were not activated by localized pinching of hairy skin in regions activated by stroking (FIG. 3f, g, i), and this selectivity was not due to desensitization (FIG. 13a-f). These data indicate that MRGPRB4⁺ fibres are activated by massage-like stroking of hairy skin. Thus, the two classes of cutaneous C fibres marked by expression of MRGPRB4 and MRGPRD, respectively, respond to distinct types of mechanical stimulation in vivo.

### Example 6

### Anxiolytic Effect of Activation of MrgprB4⁺ Neurons

The stimuli used to activate MRGPRB41 fibres were designed to mimic stroking and allogrooming stimuli. The social interactions associated with such stimuli have been shown to be positively reinforcing in juvenile mice, using a conditioned place preference (CPP) assay, suggesting that these stimuli may have a positive affective valence. To determine whether direct activation of MRGPRB41 neurons could similarly promote a preference for the location in which this stimulation occurred, a pharmacogenetic strategy was used. Juvenile (1-month-old) Mrgprb4-cre male mice were injected neonatally with an AAV encoding the hM3-(G_{q}-coupled)DREADD19, the activation of which by clozapine-N-oxide (CNO) causes membrane depolarization (FIG. 4a).

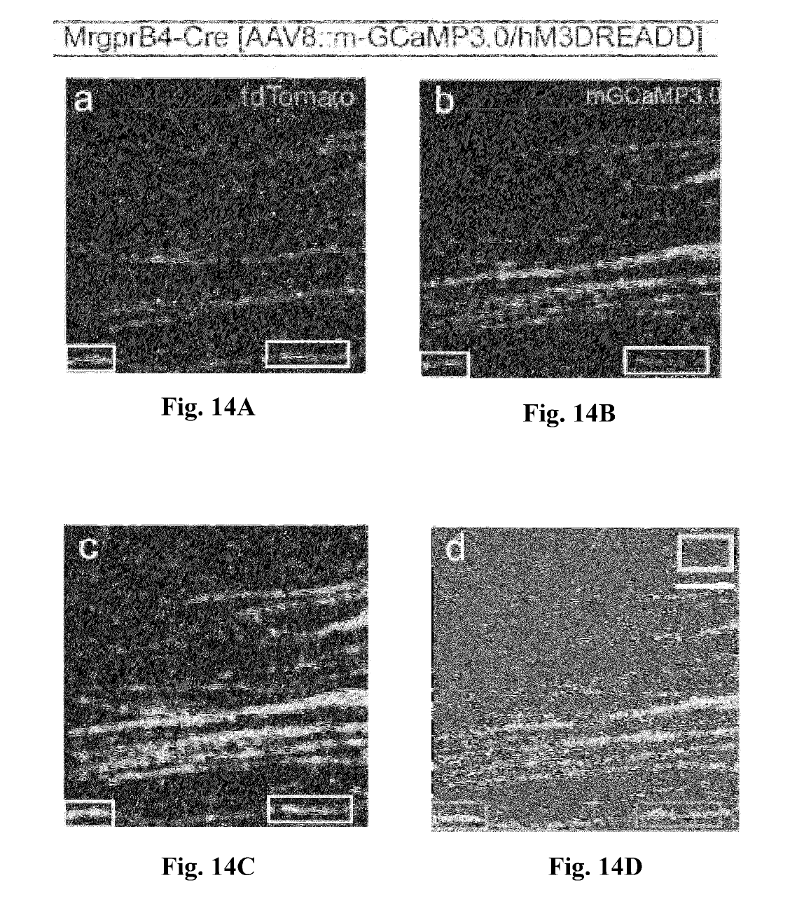

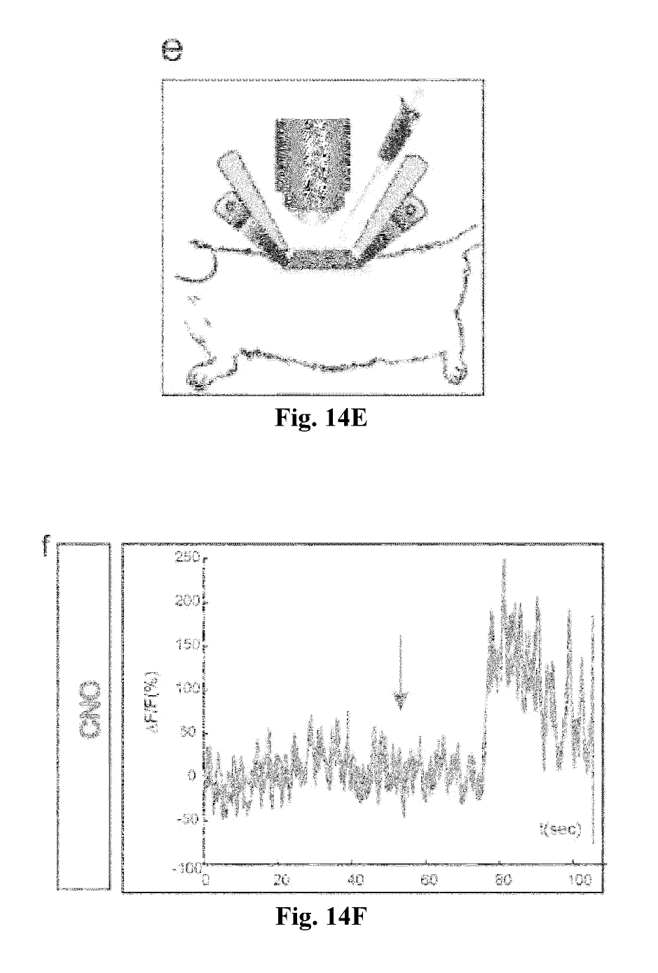

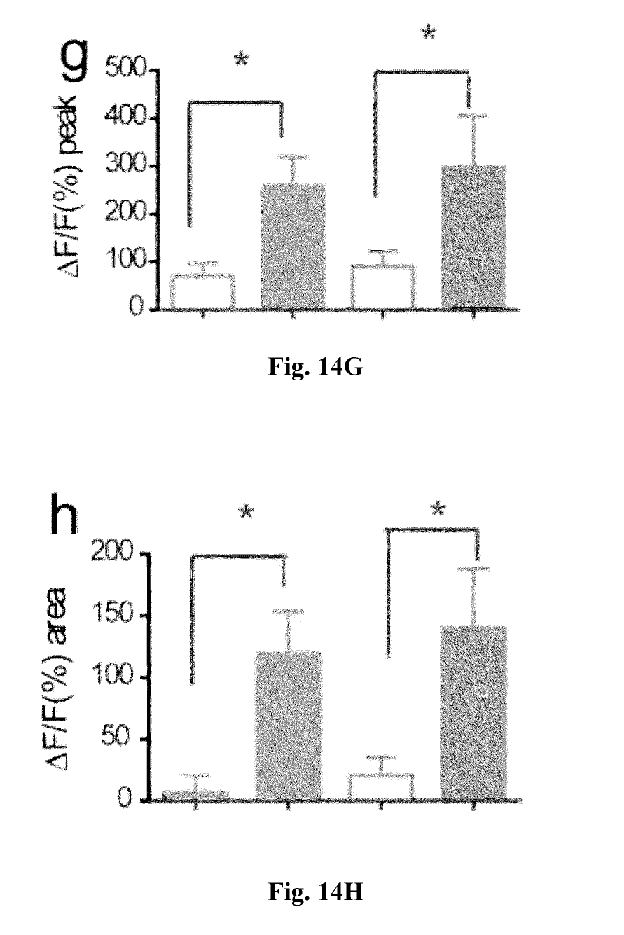

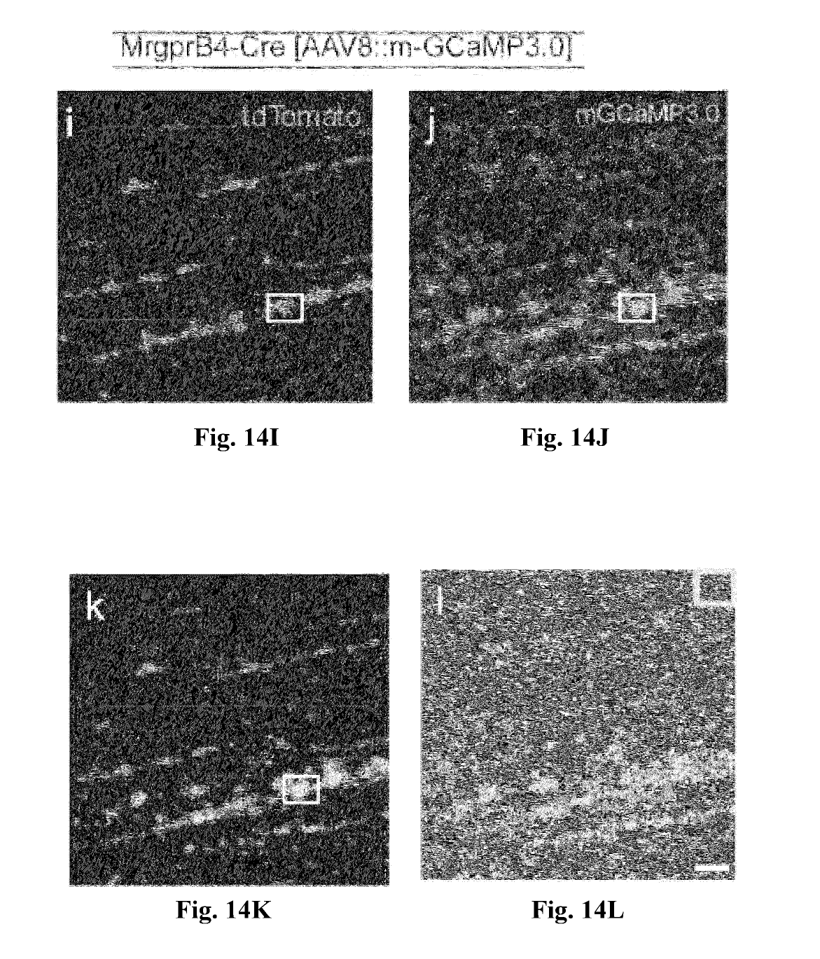

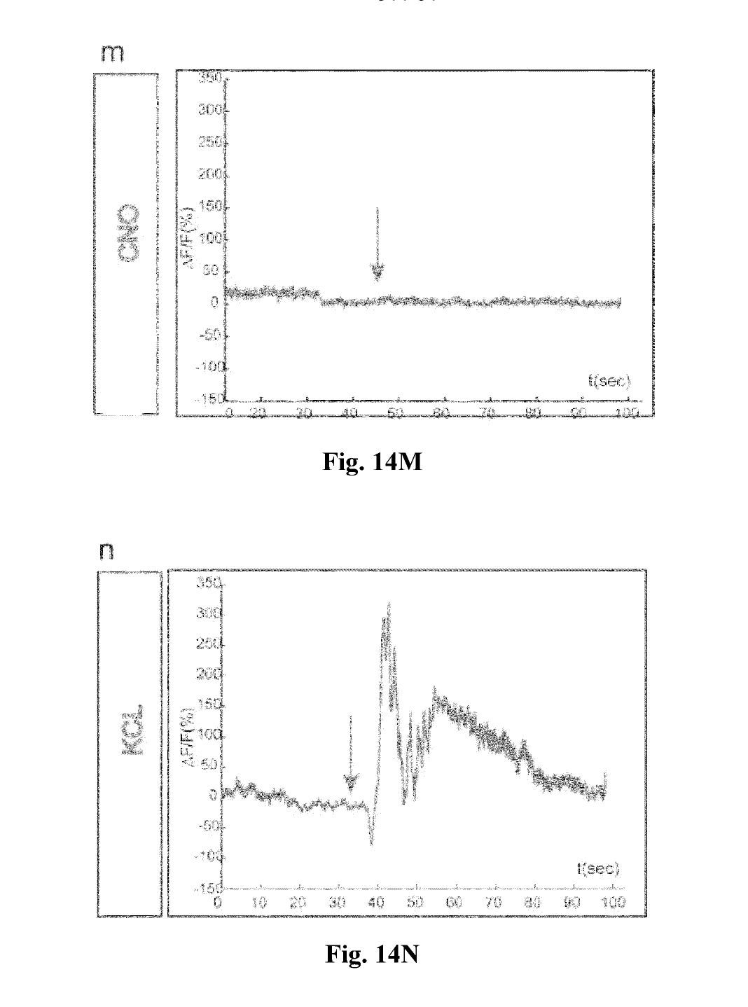

Spinal application of CNO was employed to image activation of hM3DREADD and GCaMP3.0-expressing MrgprB4⁺ fibers. The low probability of double-infection of individual MrgprB4⁺ neurons with both the GCaMP3.0 and hM3DREADD Cre-dependent viruses (˜30%), taken together with the difficulty of identifying sites for focal peripheral injection corresponding to specific MrgprB4⁺ central afferent fibers visualized in the spinal cord, precluded peripheral delivery of CNO for this assay. Thus, calcium imaging experiments confirmed that CNO was able to induce calcium transients in MRGPRB4⁺ spinal afferent fibres co-expressing GCaMP3.0 and hM3DREADD (FIG. 14).

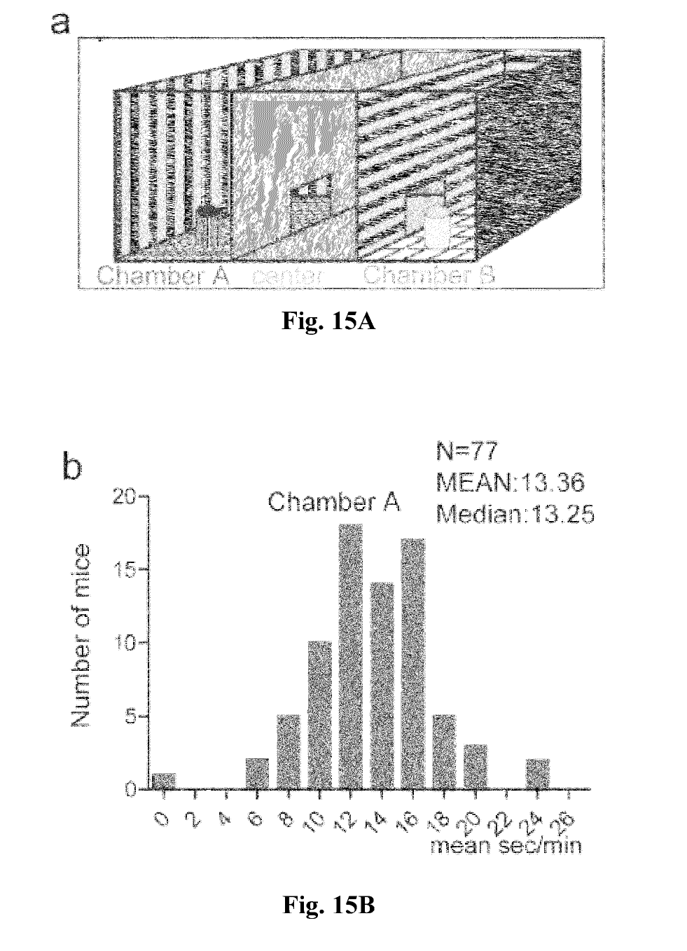

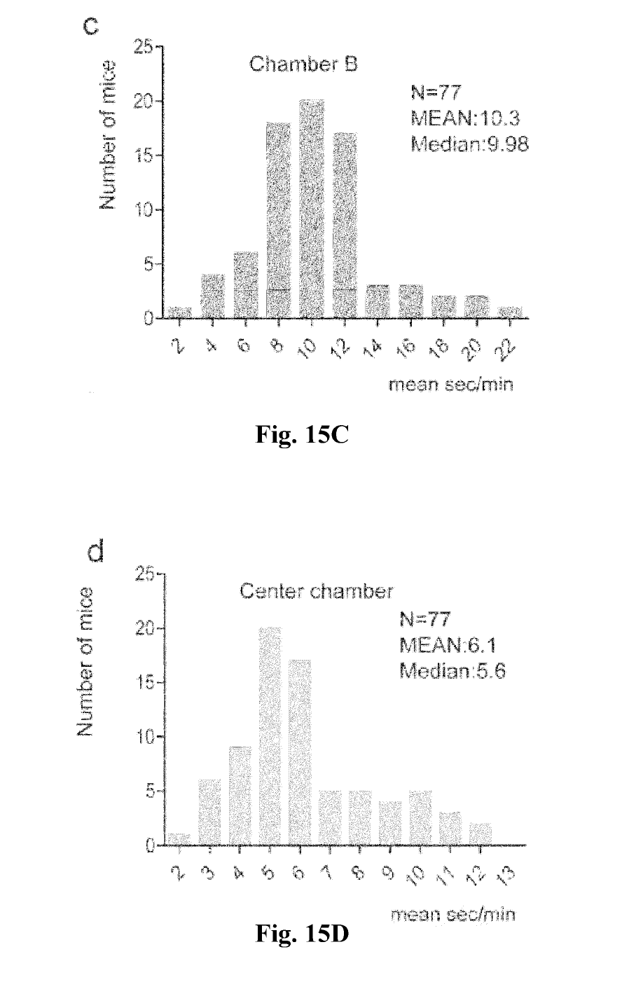

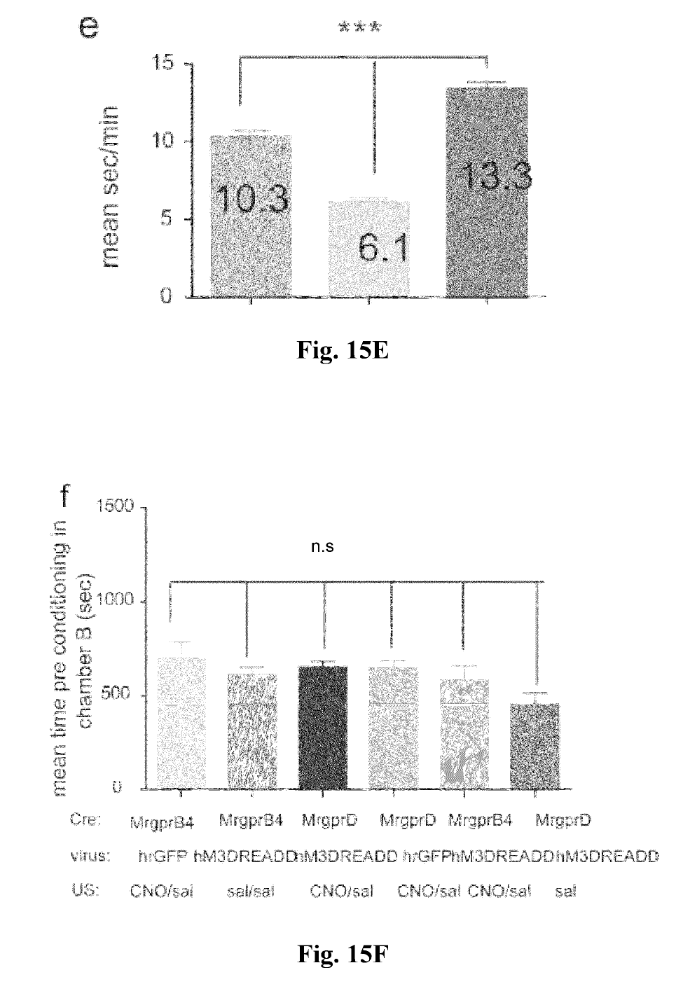

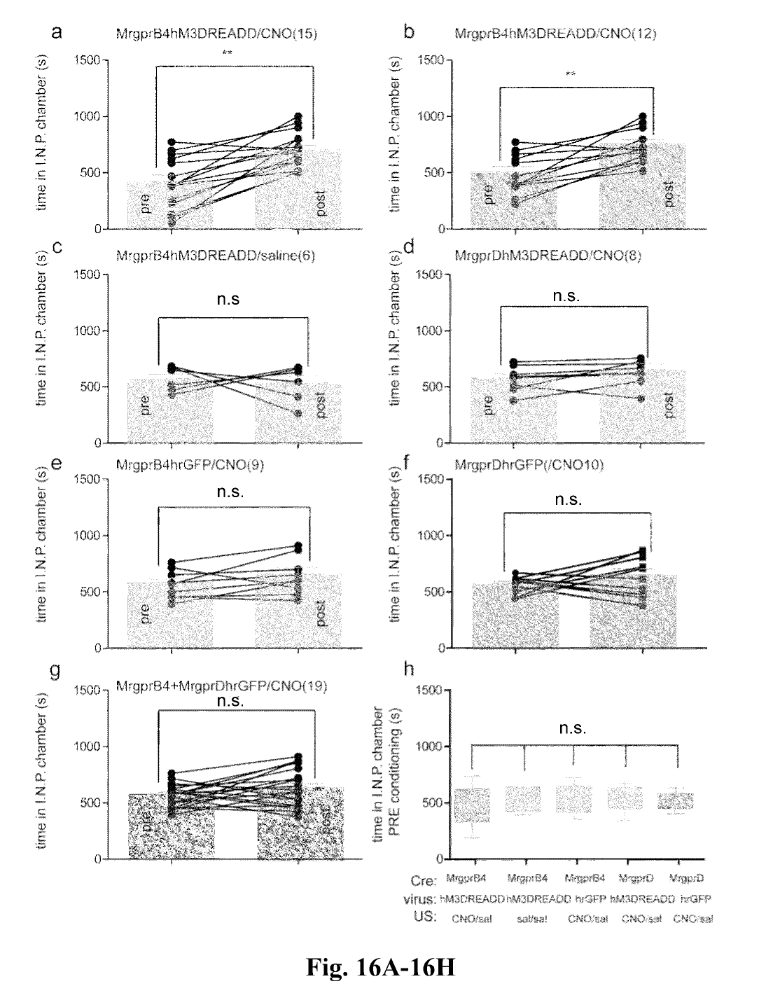

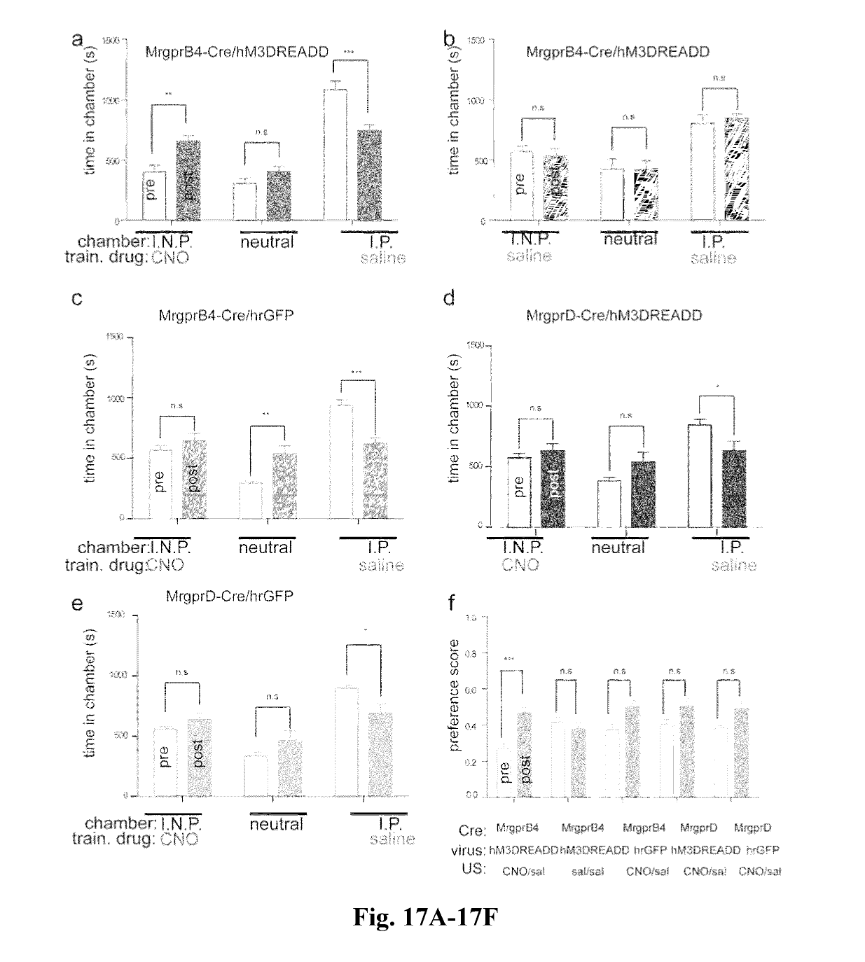

Whether activation of MRGPRB4⁺ neurons would promote a preference for the chamber associated with CNO treatment was tested. Because most mice during a pre-training exposure to the CPP apparatus showed an initial preference for one of the two side chambers (FIG. 4b, ‘I.P.’ and FIG. 15), a biased design was used to test whether activation of MRGPRB4⁺ neurons would increase the animals' preference for the initially non-preferred (I.N.P.) chamber. To do this, mice were conditioned over 4 days (experimenter blind to genotype) by pairing a 1-hr exposure to CNO with the I.N.P. chamber on each of two days, alternating with exposure to saline in the I.P. chamber (FIG. 4c, lower). When tested on the day after conditioning, Mrgprb4-hM3DREADD mice (FIG. 4e), but not a series of control mice (FIG. 4f-i), exhibited a statistically significant increase in the time they spent in the I.N.P. chamber that was paired with CNO exposure (FIG. 4c, d; 190±95% increase, P<0.01 pre-versus post-training, n=15 mice; see FIG. 16 for scatterplots; FIG. 17f). For example, MrgprB4::hM3DREADD mice showed a statistically significant positive “difference score” (time spent in the specified chamber after conditioning-before conditioning) for the CNO-paired (I.N.P.) chamber (FIG. 4e, j; 253±66 sec increase in the CNO-paired chamber vs. −340±74 sec decrease in the saline-paired chamber, p<0.01). No significant change in the difference score for the I.N.P. chamber was observed when a cohort of mice expressing a neutral reporter (hrGFP) in either MrgprB4⁺ or MrgprD⁺ neurons was conditioned with CNO (FIG. 4f, n=9 and FIG. 4i, n=10), or when mice expressing hMDREADD were conditioned using saline in both chambers (FIG. 4g, n=6; see FIG. 17b-e for absolute times spent in each chamber for each control group). A direct comparison of difference scores in the I.N.P. chamber showed that only the experimental group exhibited a statistically significant positive shift (FIG. 4j, p<0.01). The experimental, but not the control groups, also showed a statistically significant increase in their preference score for the I.N.P. chamber (time spent in the CNO/I.N.P. chamber divided by total time spent in the two test chambers; 195±86% increase, p<0.001 pre vs. post; FIG. 17f)

The mean difference score of the experimental animals in the I.N.P. chamber (FIG. 4e, j; 253±65.8) was significantly higher than that of the pooled controls (FIG. 4j; 51.8±35.3, t=2.92, P<0.01, n=33 mice) and our statistical power (0.83) was sufficient to detect this difference given the effect size (difference of means=201±70; 95% confidence interval: 62.9-339.3; Cohen's d=0.872). There was no statistically significant difference between groups in the time spent in the I.N.P. chamber during the pre-test (FIG. 16). These data suggest that artificial activation of MRGPRB4⁺ neurons in vivo is positively reinforcing and/or anxiolytic. In contrast, artificial activation of MRGPRD⁺ neurons using CNO and DREADD produced neither CPP (FIG. 4d, h and FIGS. 16d and 17d) nor (in separate experiments) conditioned place aversion (CPA; data not shown).

Examples 3-6 show the first application of calcium imaging to record physiological response of primary sensory neurons to cutaneous stimulation in an intact animal. Using genetically encoded calcium sensors, a molecularly defined subpopulation of unmyelinated fibres that responds to innocuous stroking of hairy skin in vivo was identified. Selective manipulation of these neurons in vivo also provides the first example of a genetically identified population of C fibres whose function activation has a positive rather than negative behavioral valence.

The inability to detect activation of MRGPRB4⁺ neurons by mechanical stimuli in isolated skin-nerve preparations, distinguishes MRGPRB4⁺ neurons functionally from other populations of unmyelinated mechanosensitive neurons that have been recently characterized in this manner. Without being bound to a particular theory, it is believed that the inability to detect activation of MrgprB4⁺ neurons by mechanical stimuli ex vivo could be due to shaving the skin in such preparations, which may reduce the mechanical force that can be applied via bending of hairs during stroking, or more likely to the absence of underlying connective tissue, dermis and musculature in the skin explant, which makes it impossible to apply the same force or deformation as can be applied in vivo.

### Example 7

### Identification of MrgprB4 Agonists

This example illustrates the identification of MrgprB4 agonists.

Compounds to be tested for the potential to be effective for activating MrgprB4 are provided. As discussed above, the compounds may be, without limitation, small molecules (including both organic and inorganic molecules), peptides, peptide mimetics, nucleic acids, or antibodies.

In some embodiments, the compounds are initially screened for their ability to interact with MrgprB4. The candidate MrgprB4 agonist that binds to MrgprB4 is then administered to mammalian cells, such as HEK293 cells, stably expressing MrgprB4 gene in an intracellular calcium mobilization assay with the fluorometric imaging plate reader (FLIPR, Molecular Devices). The cells are monitored and measured for level of cell fluorescence, which indicates the extent of activation of MrgprB4 receptor. A successful MrgprB4 agonist is able to induce cell fluorescence to a level substantially comparable or higher in comparison to cells that is exposed to a control known MrgprB4 agonist (for example, ATP).

In some embodiments, the compounds are tested for their ability to modulate the level of MrgprB4 gene expression, preferably increasing the level of transcription of MrgprB4 gene. The level of transcription of MrgprB4 gene can be determined by measuring the level of MrgprB4 mRNA or MrgprB4 protein. The preferred MrgprB4 agonists significantly increase the level of MrgprB4 gene expression. In some embodiments, compounds are tested for their ability to enhance the level of MrgprB4 protein in cells. The level of MrgprB4 protein in cells can be determined by conventional techniques such as western blot. The preferred MrgprB4 agonists significantly increase the level of MrgprB4 protein in cells.

In some embodiments, the compounds are tested for their ability to positively allosterically modulate the activation of MrgprB4. The compounds are initially screened for their ability to interact with MrgprB4. A known MrgprB4 agonist (for example ATP) is administered to mammalian cells, such as HEK293 cells, stably expressing MrgprB4 gene in a low concentration. The cells are monitored and measured for level of cell fluorescence, which indicates the extent of activation of MrgprB4 receptor. The candidate MrgprB4 agonist that binds to MrgprB4 is then added to the cells in the presence of the low concentration of known MrgprB4 and tested for its positive allosteric modulation using a concentration-response (C/R) curve method. A successful MrgprB4 agonist acting as a positive allosteric modulator is able to significantly increase the amount of cell fluorescence triggered by the binding of low concentration of known MrgprB4 agonist (such as ATP) to MrgprB4 receptor.

### Example 8

### Identification of Therapeutics for the Treatment of Anxiety

This example illustrates the identification of compounds that can be used to treat, prevent, or ameliorate anxiety.

Compounds to be tested for effective therapeutics for anxiety are provided. As discussed above, the compounds can be, without limitation, small molecules (including both organic and inorganic molecules), peptides, peptide mimetics, nucleic acids, or antibodies. In some embodiments, the compounds are initially screened for their ability to activate MrgprB4. Compounds that are able to activate MrgprB4 can then tested for their ability to activate MrgprB4⁺ neurons. And the compounds that are activators of MrgprB4⁺ neurons can be used for treating, preventing, or ameliorating anxiety.

### Example 9

### Treatment of Anxiety

This example illustrate the treatment of a patient suffering from or at risk of developing anxiety.

A patient suffering from or at risk of developing anxiety is identified and administered an effective amount of a pharmaceutical composition comprising one or more activators of MrgprB4⁺ neurons. A typical daily dose for an activator of MrgprB4⁺ neurons can range from about 0.01 μg/kg to about 1 mg/kg of patient body weight or more per day, depending on the factors mentioned above, preferably about 10 μg/kg/day to about 100 μg/kg/day. The appropriate dosage and treatment regimen can be readily determined by one of ordinary skill in the art based on a number of factors including the nature of the activators of MrgprB4⁺ neurons used, the route of administration and the patient's disease state. Treatment efficacy is evaluated by observing delay or slowing of disease progression, amelioration or palliation of the disease state, and/or remission.

The foregoing description and examples detail certain preferred embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof. Although the present application has been described in detail above, it will be understood by one of ordinary skill in the art that various modifications can be made without departing from the spirit of the invention.

In this application, the use of the singular can include the plural unless specifically stated otherwise or unless, as will be understood by one of skill in the art in light of the present disclosure, the singular is the only functional embodiment. Thus, for example, “a” can mean more than one, and “one embodiment” can mean that the description applies to multiple embodiments. Additionally, in this application, “and/or” denotes that both the inclusive meaning of “and” and, alternatively, the exclusive meaning of “or” applies to the list. Thus, the listing should be read to include all possible combinations of the items of the list and to also include each item, exclusively, from the other items. The addition of this term is not meant to denote any particular meaning to the use of the terms “and” or “or” alone. The meaning of such terms will be evident to one of skill in the art upon reading the particular disclosure.

All references cited herein including, but not limited to, published and unpublished patent applications, patents, text books, literature references, and the like, to the extent that they are not already, are hereby incorporated by reference in their entirety. To the extent that one or more of the incorporated literature and similar materials differ from or contradict the disclosure contained in the specification, including but not limited to defined terms, term usage, described techniques, or the like, the specification is intended to supersede and/or take precedence over any such contradictory material.

The term “comprising” as used herein is synonymous with “including,” “containing,” or “characterized by,” and is inclusive or open-ended and does not exclude additional, unrecited elements or method steps.

What is claimed is:

1. A method of identifying compounds having anxiolytic activities, comprising:
(a) providing a candidate compound;
(b) testing the candidate compound for its ability to activate MrgprB4⁺ neurons; and
(c) testing the candidate compound for its activity to stimulate positive valence behavior in a subject if the candidate compound activates MrgprB4⁺ neurons in step (b).

2. The method of claim 1, wherein step (b) is carried out in vitro.

3. The method of claim 2, wherein step (b) is carried out in a skin-nerve culture.

4. The method of claim 1, wherein the candidate compound is an MrgprB4 agonist.

5. The method of claim 1, wherein step (b) is carried out in vivo.

6. The method of claim 5, wherein the candidate compound is administered to the subject via injection.

7. The method of claim 6, wherein the candidate compound is injected into spinal cord of the animal or via peripheral injection into the skin of the subject.

8. The method of claim 1, wherein step (b) comprises performing calcium imaging in MrgprB4⁺ neurons.

9. The method of claim 1, wherein step (c) comprises testing the candidate compound using a conditioned place preference assay.

10. The method of claim 9, wherein step (c) comprises determining conditioned place aversion.

11. The method of claim 1, wherein step (c) comprises applying the candidate compound peripherally on the subject.

12. The method of claim 11, wherein the candidate compound is applied topically on the subject.

13. The method of claim 12, wherein the candidate compound is in a topical composition selected from the group consisting of lotion, cream, foam, ointment, gel, transdermal patch, powder, and spray.

14. The method of claim 1, wherein the candidate compound is a small molecule, peptide, or nucleic acid.

15. A method of treating anxiety in a subject, comprising:
identifying a subject suffering from anxiety; and
administering to the subject an effective amount of an activator for MrgprB4⁺ neurons.

16. The method of claim 15, additionally comprising the step of identifying an activator for MrgprB4⁺ neurons.

17. The method of claim 15, wherein the activator for MrgprB4⁺ neurons is an agonist for MrgprB4⁺ receptor.

18. The method of claim 15, wherein the activator for MrgprB4⁺ neurons is topically administered to the subject.

19. The method of claim 15, where the activator for MrgprB4⁺ neurons is a small molecule, a peptide, or a nucleic acid.

20. The method of claim 15, wherein the anxiety is caused by itching or pain.

21. A method for identifying activating stimuli for sensory neurons, comprising
applying a stimulus to a subject, wherein the subject has a population of a subset of sensory neurons; and
performing two-proton calcium imaging to determine activation of the subset of sensory neurons.

22. The method of claim 21, wherein the sensory neurons are MrgprB4⁺ neurons.

23. The method of claim 21, wherein the population of a subset of sensory neurons is genetically modified.

24. The method of claim 23, wherein the genetic modification is carried out by intra-peritoneally injecting a viral vector to neonatal pups of the subject.

25. The method of claim 24, where the viral vector is derived from adeno-associated virus of serotype 8 (AAV8).

26. The method of claim 24, wherein the viral vector comprises two portions of MrgprB4 open reading frame, wherein the two portions of MrgprB4 open reading frame are separated by a nucleic acid sequence encoding one or more marker genes.

27. The method of claim 23, wherein the neonatal pups of the subject inducibly express Cre recombinase in ganglia.

28. The method of claim 27, wherein the neonatal pups of the subject inducibly express Cre recombinase in MrgprB4⁺ neurons.

29. The method of claim 21, wherein the stimulus is a mechanical stimulus, a thermal stimulus, a chemical stimulus, or a combination thereof.

30. The method of claim 21, wherein the stimulus is applied centrally or peripherally to the subject.

31. The method of claim 21, wherein the stimulus is pinching, massaging, grooming, stroking, or brushing.

32. The method of claim 21, wherein two-proton calcium imaging is carried out on the skin of the subject.


SEQUENCE LISTING (2 sequences)

SEQ ID NO: 1 - protein, 8 aa, Artificial Sequence
  MARCKS sequence
MGCCFSKT

SEQ ID NO: 2 - protein, 321 aa, Mus musculus
MGTTTLAWNINNTAENGSYTEMFSCITKFNTLNFLTVIIAVVGLAGNGIVLWLLAFHLHRNAFSVYVLNLAGADFLYLFTQVVHSLECVLQLDNNSFYILLIVTMFAYLAGLCMIAAISAERCLSVMWPIWYHCQRPRHTSAIMCALVWVSSLLLSLVVGLGCGFLFSYYDYYFCITLNFITAAFLIVLSVVLSVSSLALLVKIVWGSHRIPVTRFFVTIALTVVVFIYFGMPFGICWFLLSRIMEFDSIFFNNVYEIIEFLSCVNSCANPIIYFLVGSIRQHRLRWQSLKLLLQRAMQDTPEEESGERGPSQRSGELETV